(19) United States
(12) Patent Application Publication
(10) Pub. No.: US 2004/0067955 A1
(43) Pub. Date: Apr. 8, 2004

(54) Pyridazinone compound and pharmaceutical use thereof

(75) Inventors: Seiichiro Tabuchi, Osaka (JP); Hideo Tsutumi, Osaka (JP); Atsushi Akahane, Osaka (JP); Yoshinari Sato, Takaishi-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Fujisawa Pharmaceutical Co. Ltd., Osaka-shi (JP)

(21) Appl. No.: 10/653,129

(22) Filed: Sep. 3, 2003

(30) Foreign Application Priority Data
Sep. 6, 2002 (AU) 2002951245
Oct. 24, 2002 (AU) 2002952245

(51) Int. Cl. A61K 31/501; A61K 31/444; C 07D 4 3/04; C 07D 4 1/04

(52) U.S. Cl. 514/252.03; 514/334; 544/238; 546/261

(57) ABSTRACT

A pyridazinone or pyridone compound of the following formula (I).

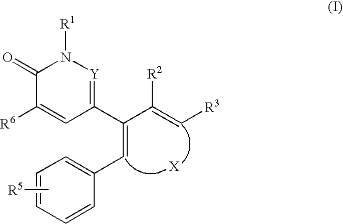

wherein

X is

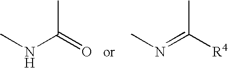

Y is N or CH

or a salt thereof.

The pyridazinone or pyridone compound (I) and a salt thereof of the present invention are adenosine antagonists and are useful for the prevention and/or treatment of depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure and the like.

### TECHNICAL FIELD

The present invention relates to a novel pyridazinone or pyridone compound, preferably a pyridylpyridazinone compound, and a salt thereof, which are useful as medicaments.

### BACKGROUND ART

Some pyrazolopyridyl pyridazinone compounds to be useful as remedy for renal failure, heart failure, depression and the like are known (e.g. WO 95/18128, WO 98/03507, WO 00/24742, etc.).

2-Aminopyridine compounds to exhibit adenosine receptor antagonism are known (WO 02/14282).

6-(2-Phenyl-3-pyridyl)-3(2H)-pyridazinone compounds and derivatives thereof are novel, so there has been no knowledge about these compounds. In addition, any pyridylpyridazinone compounds having both of adenosine A₁ and A₂ₐ inhibitory activities are not known.

### DISCLOSURE OF INVENTION

The present invention relates to a novel pyridazinone or pyridone compound, preferably a pyridylpyridazinone compound, and a pharmaceutically acceptable salt thereof, which are useful as medicaments; processes for the preparation of said pyridazinone or pyridone compound and a salt thereof; a pharmaceutical composition comprising, as an active ingredient, said pyridazinone or pyridone compound or a pharmaceutically acceptable salt thereof; a use of said pyridazinone or pyridone compound or a pharmaceutically acceptable salt thereof as a medicament; and a method for using said pyridazinone or pyridone compound or a pharmaceutically acceptable salt thereof for therapeutic purposes, which comprises administering said pyridazinone or pyridone compound or a pharmaceutically acceptable salt thereof to a human being or an animal.

The pyridazinone or pyridone compound and a salt thereof are adenosine antagonists (especially, A₁ receptor and A₂ (particularly A₂ₐ) receptor dual antagonists) and possess various pharmacological actions such as anticatalepsy action, cognitive enhancing action, analgesic action, locomotor action, antidepressant action, diuretic action, cardioprotective action, cardiotonic action, vasodilating action (e.g. cerebral vasodilating action, etc.), the action of increasing the renal blood flow, renal protective action, improvement action of renal function, enhancing action of lipolysis, inhibition action of anaphylactic bronchoconstriction, acceleration action of the insulin release, the action of increasing the production of erythropoietin, inhibiting action of platelet aggregation, or the like.

They are useful as cognitive enhancer, antianxietry drug, antidementia drug, psychostimulant, analgesic, cardioprotective agent, antidepressant, ameliorants of cerebral circulation, tranquilizer, drug for heart failure, cardiotonic agent, antihypertensive agent, drug for renal failure (renal insufficiency), drug for renal toxicity, renal protective agent, drug for improvement of renal function, diuretic, drug for edema, antiobesity, antiasthmatic, bronchodilator, drug for apnea, drug for gout, drug for hyperuricemia, drug for sudden infant death syndrome (SIDS), ameliorants of immunosuppressive action of adenosine, antidiabetic agent, drug for ulcer, drug for pancreatitis, drug for Meniere's syndrome, drug for anemia;

drug for thrombosis, drug for myocardial infarction, drug for obstruction, drug for arteriosclerosis obliterans, drug for thrombophlebitis, drug for cerebral infarction, drug for transient ischemic attack, drug for angina pectoris, or the like;

and useful for the prevention and/or treatment of depression, dementia (e.g. Alzheimer's disease, cerebrovascular dementia, dementia accompanying Parkinson's disease, etc.), Parkinson's disease, anxiety, pain, cerebrovascular disease (e.g. stroke, etc.), heart failure;

hypertension (e.g. essential hypertension, nephrogenous hypertension, etc.);

circulatory insufficiency (acute circulatory insufficiency) caused by, for example, ischemia/reperfusion injury (e.g. myocardial ischemia/reperfusion injury, cerebral ischemia/reperfusion injury, peripheral ischemia/reperfusion injury, etc.), shock (e.g. endotoxin shock, hemorrhagic shock, etc.), surgical procedure, or the like; post-resuscitation asystole;

bradyarrhythmia;

electromechanical dissociation;

hemodynamic collapse;

SIRS (systemic inflammatory response syndrome);

multiple organ failure;

renal failure (renal insufficiency) (e.g. acute renal failure, etc.), renal toxicity [e.g. renal toxicity induced by a drug such as cisplatins, gentamicin, FR-900506 (disclosed in EP-0184162), cyclosporin (e.g. cyclosporinA) or the like; glycerol, etc.], nephrosis, nephritis, edema (e.g. cardiacedema, nephrotic edema, hepatic edema, idiopathic edema, drug edema, acute angioneurotic edema, hereditary angioneurotic edema, carcinomatous ascites, gestational edema, etc.);

obesity, bronchial asthma, gout, hyperuricemia, sudden infant death syndrome, immunosuppression, diabetes, ulcer such as peptic ulcer (e.g. gastric ulcer, duodenal ulcer, etc.), pancreatitis, Meniere's syndrome, anemia, dialysis-induced hypotension, constipation, ischemic bowel disease, ileus (e.g. mechanical ileus, adynamic ileus, etc.); and

myocardial infarction, thrombosis (e.g. arterial thrombosis, cerebral thrombosis, etc.), obstruction, arteriosclerosis obliterans, thrombophlebitis, cerebral infarction, transient ischemic attack, angina pectoris, or the like.

The novel pyridazinone or pyridone compound of the present invention can be shown by the following formula (I).

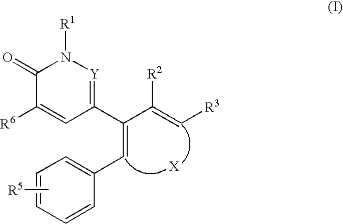

wherein

X is

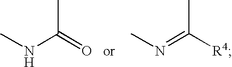

Y is N or CH;

R¹ is hydrogen or optionally substituted lower alkyl;

R² is hydrogen or halogen;

R³ is hydrogen, lower alkyl, lower alkoxy, halogen, hydroxy, cyano, amino, carbamoyl, thiocarbamoyl, aryl, acyl, acylamino or heterocyclic group,

each of which may be optionally substituted;

R⁴ is hydrogen, lower alkyl, lower alkoxy, halogen, hydroxy, cyano, amino, carbamoyl, acyl, acylamino or

—A—R⁷

wherein

A is —CH═CH— or —CH═N—, and

R⁷ is lower alkyl, lower alkoxy, hydroxy, cyano, acyl, aryl(lower)alkoxy or acyloxy,

each of which may be optionally substituted;

R⁵ is hydrogen, lower alkyl, lower alkoxy, halogen, hydroxy, each of which may be optionally substituted; and

R⁶ is hydrogen or halogen;

or a salt thereof.

The preferred embodiments of the pyridazinone or pyridone compound of the present invention represented by the general formula (I) are as follows.

(1) The pyridazinone compound of the general formula (I)

wherein

Y is N;

R¹ is hydrogen, lower alkyl, aryl(lower)alkyl, or

—(CH₂)ₙ—CO—R⁸

wherein

n is 1 or 2, and

R⁸ is hydroxy, lower alkoxy, aryl, amino, lower alkylamino, hydroxy(lower)alkylamino or optionally substituted heterocyclic group;

R² is hydrogen;

R³ is hydrogen, lower alkyl, hydroxy(lower)alkyl, lower alkoxy, amino(lower)alkoxy, halogen, hydroxy, cyano, amino, carboxy, lower alkylaminocarbonyl, lower alkanoyl, lower alkoxycarbonyl, lower alkoxycarbonylamino, carbamoyl(lower)alkoxy, optionally subsituted heterocyclic group or optionally substituted heterocyclic carbonyl;

R⁴ is hydrogen, lower alkyl, lower alkoxy, optionally substituted amino(lower)alkoxy, halogen, hydroxy, cyano, amino, hydrazino, carbamoyl, carbamoyl (lower) alkoxy, carboxy, lower alkanoyl, lower alkoxycarbonyl, aryl(lower)alkylamino, heterocyclic(lower)alkylamino, heterocyclic(lower)alkoxy, or

—NH—CO—R⁹

wherein

R⁹ is lower alkyl, lower alkoxy, aryl or heterocyclic group;

R⁵ is hydrogen, lower alkoxy, hydroxy or halogen; and

R⁶ is hydrogen;

or a salt thereof.

(2) The pyridone compound of the general formula (I)

wherein

Y is CH;

R¹ is hydrogen or lower alkyl,

R² is hydrogen or halogen;

R³ is hydrogen, halogen or amino;

R⁴ is hydrogen, halogen or amino;

R⁵ is hydrogen; and

R⁶ is hydrogen or halogen;

or a salt thereof.

(3) The pyridazinone compound of (1) above

wherein

X is

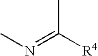

R¹ is hydrogen, lower alkyl;

R³ is hydrogen, hydroxy(lower)alkyl, halogen, hydroxy, amino, lower alkylaminocarbonyl, lower alkoxycarbonyl, optionally subsituted heterocyclic group or optionally substituted heterocyclic carbonyl; and

R⁴ is hydrogen, halogen, amino, hydrazino, aryl(lower)alkylamino, heterocyclic(lower)alkylamino, heterocyclic(lower)alkoxy, or

—NH—CO—R⁹

wherein

R⁹ is lower alkyl, aryl or heterocyclic group;

or a salt thereof.

(4) The pyridazinone compound of (3) above

wherein

R¹ is hydrogen, methyl, ethyl or isopropyl;

R³ is hydrogen, hydroxymethyl, chloro, bromo, iodo, hydroxy, methoxycarbonyl, methylthiazolyl or methylpyrazolyl;

R⁴ is hydrogen, chloro, bromo, iodo, amino, hydrazino, benzylamino, pyridylmethyl, acetamido, tert-butylcarbonylamino or benzoylamino; and

R⁵ is hydrogen, methoxy, hydroxy, fluoro, chloro, bromo or iodo;

or a salt thereof.

(5) The pyridone compound of (2) above

wherein

R¹ is isopropyl,

R² is hydrogen or chloro;

R³ is hydrogen, chloro or amino;

R⁴ is chloro or amino;

R⁶ is hydrogen or chloro;

or a salt thereof.

(6) The pyridazinone compound of (4) above

wherein

R¹ is isopropyl;

R³ is hydrogen, chloro, hydroxy, methoxycarbonyl or methylthiazolyl;

R⁴ is hydrogen, chloro, amino, hydrazino, benzylamino, pyridylmethyl, acetamido or benzoylamino; and

R⁵ is hydrogen, hydroxy, fluoro or chloro;

or a salt thereof.

The term “optionally substituted” refers to “unsubstituted or substituted by one or more suitable substituent(s)”.

The object compound (I) and a salt thereof of the present invention can be prepared by the following processes.

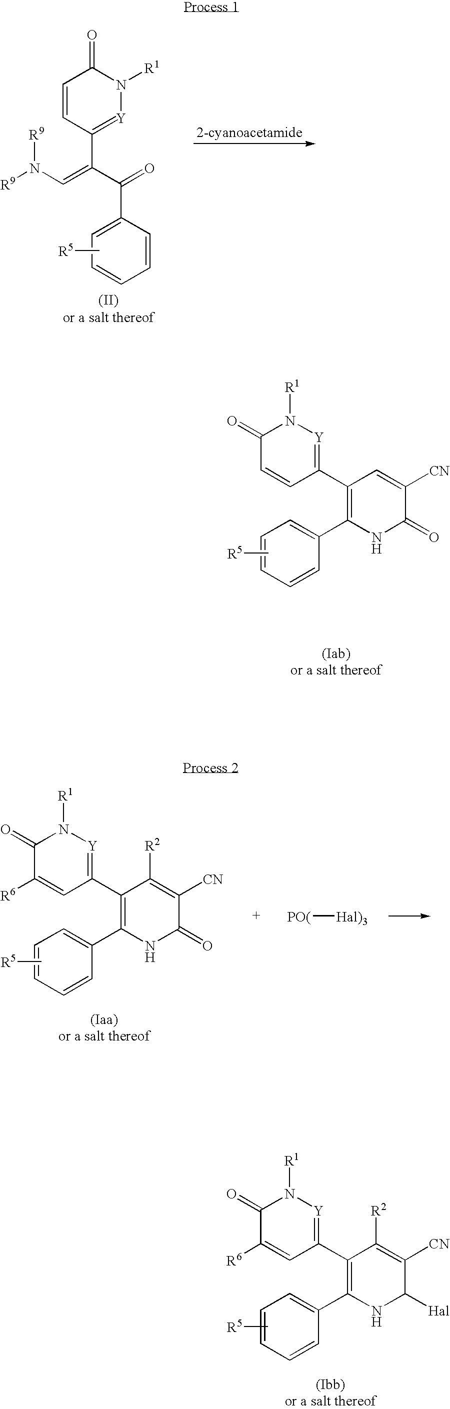

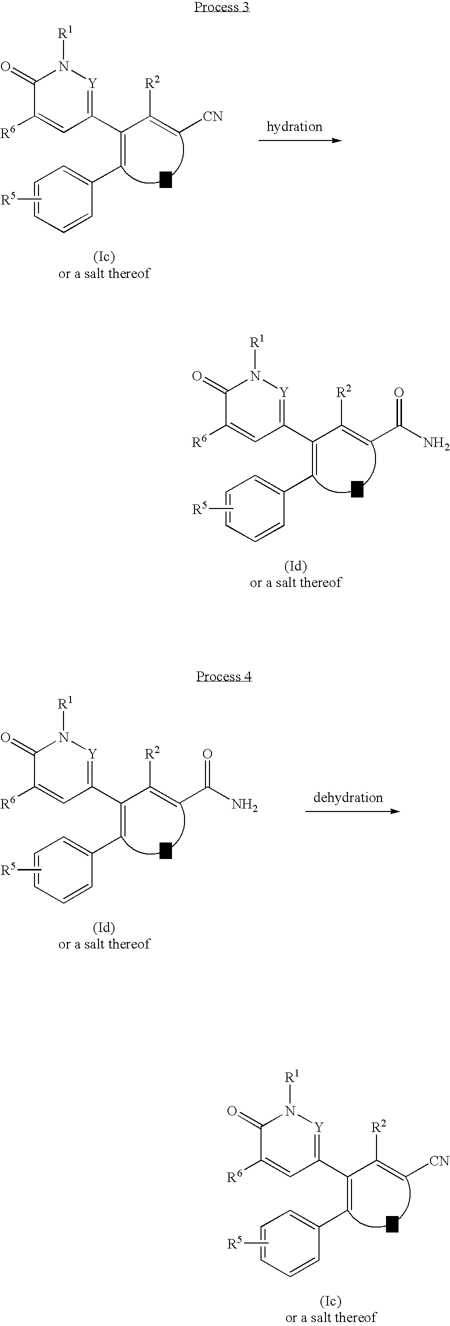

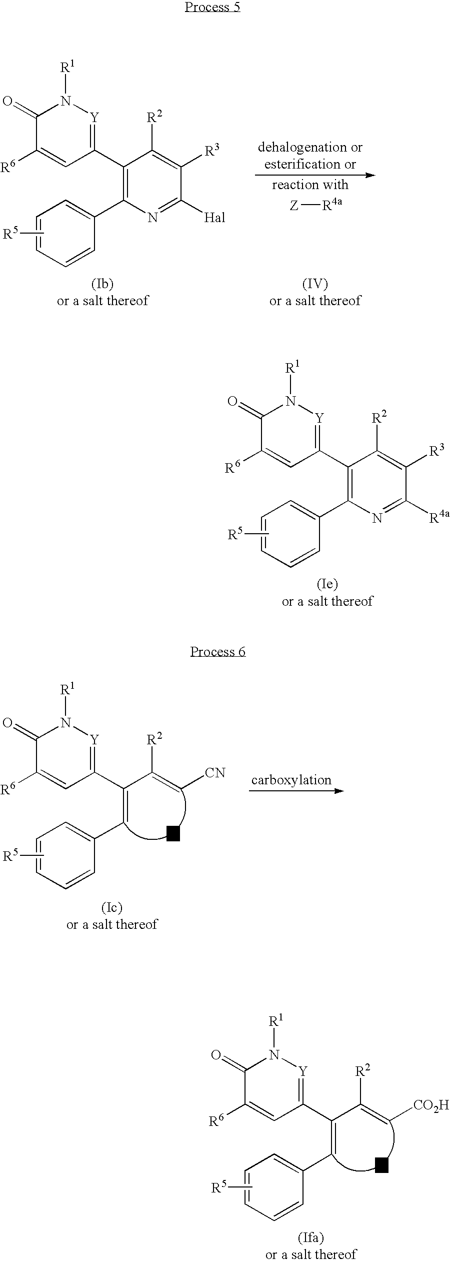

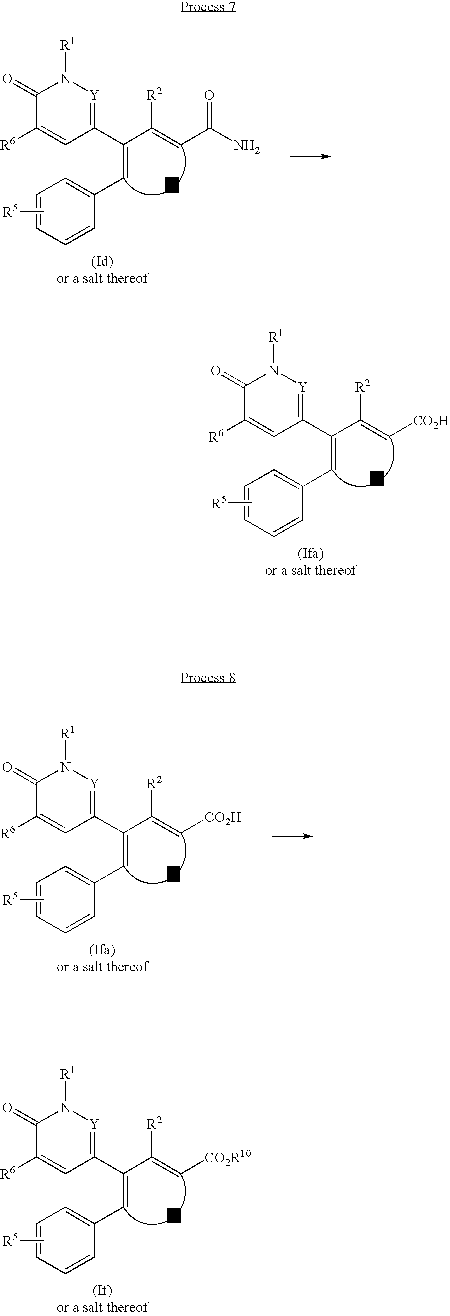

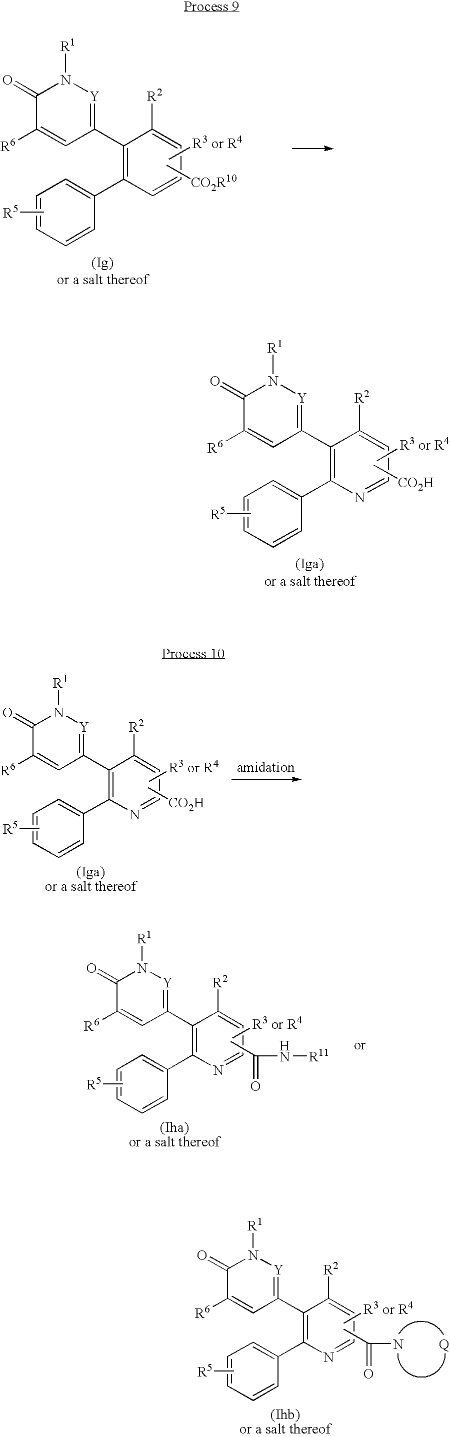

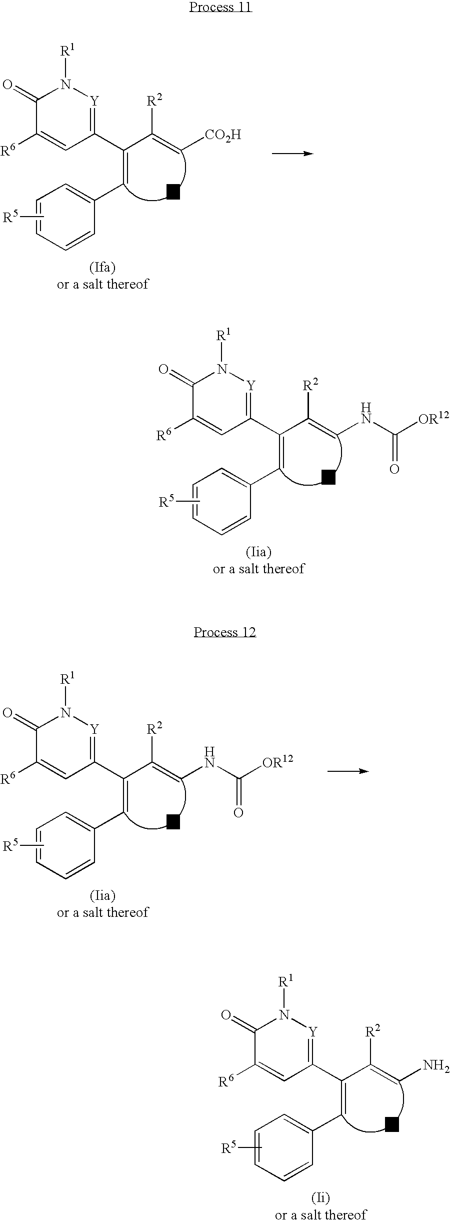

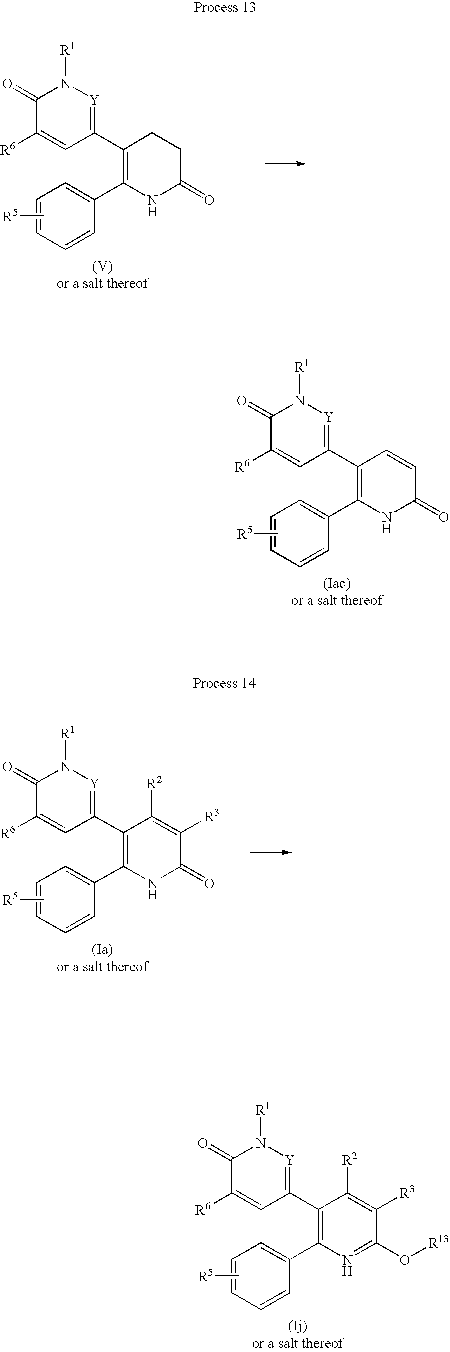

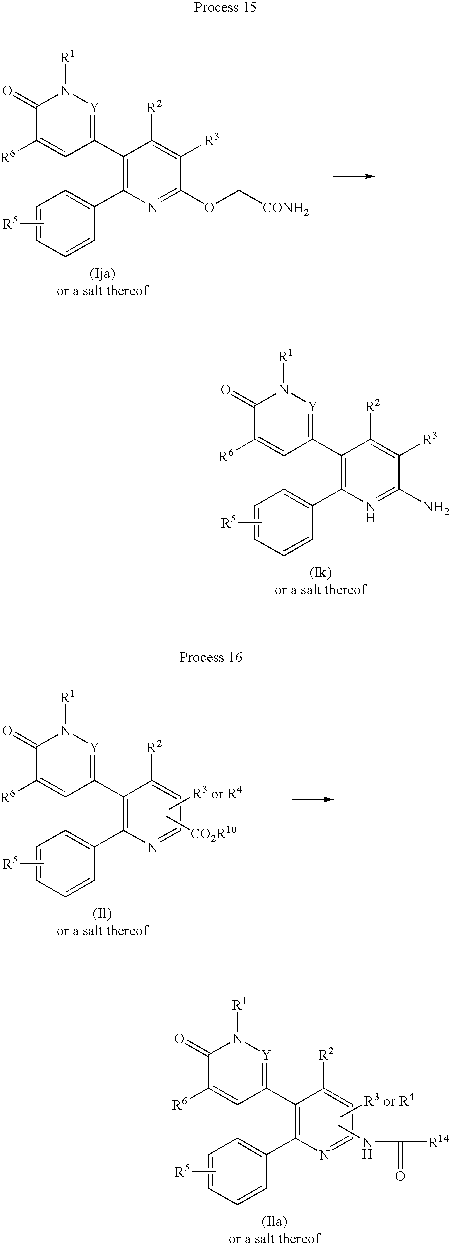

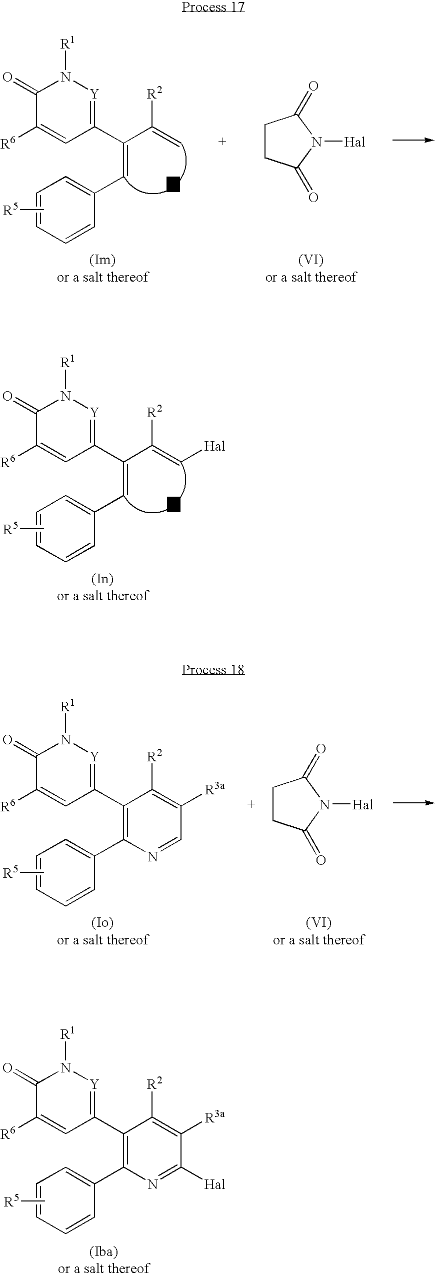

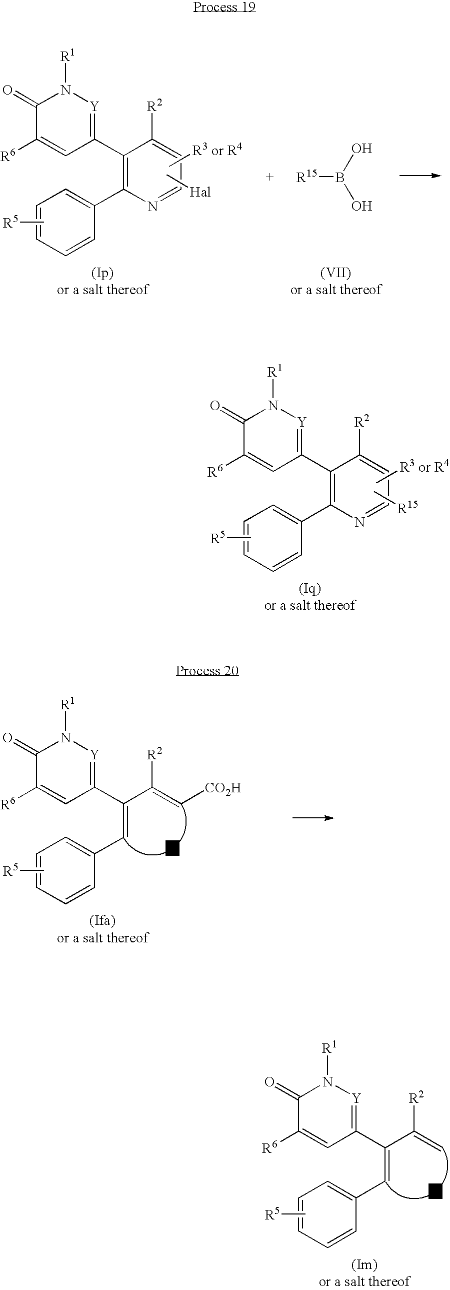

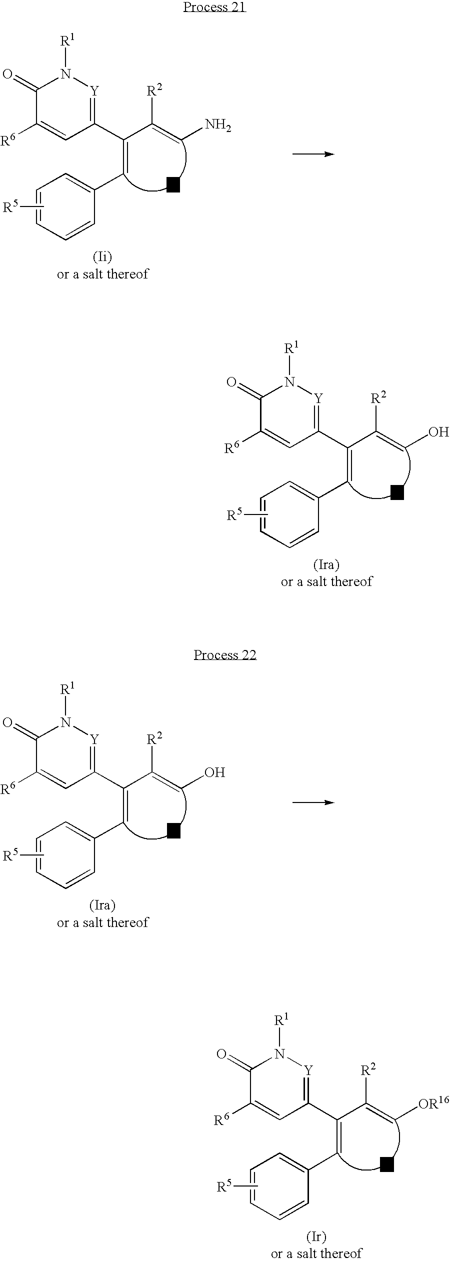

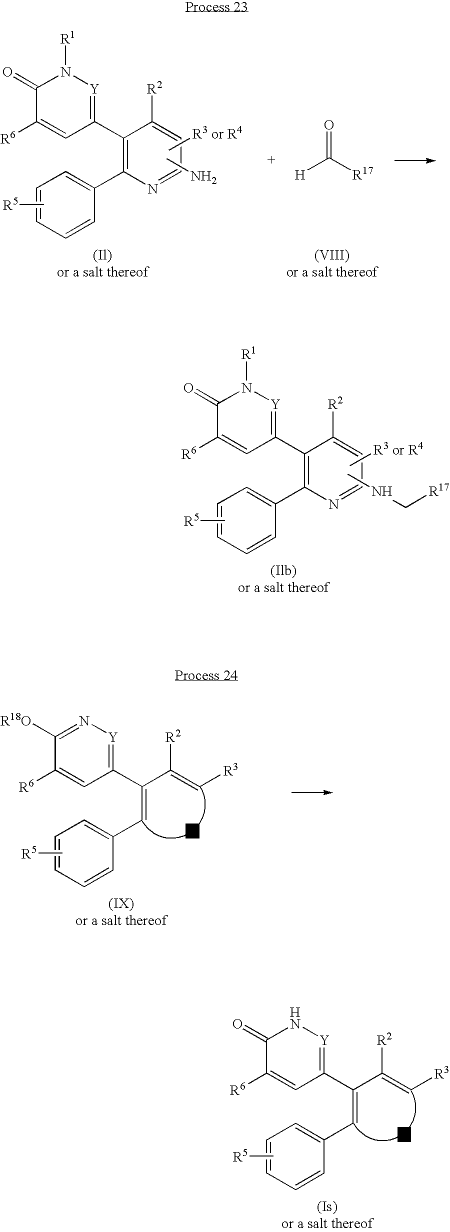

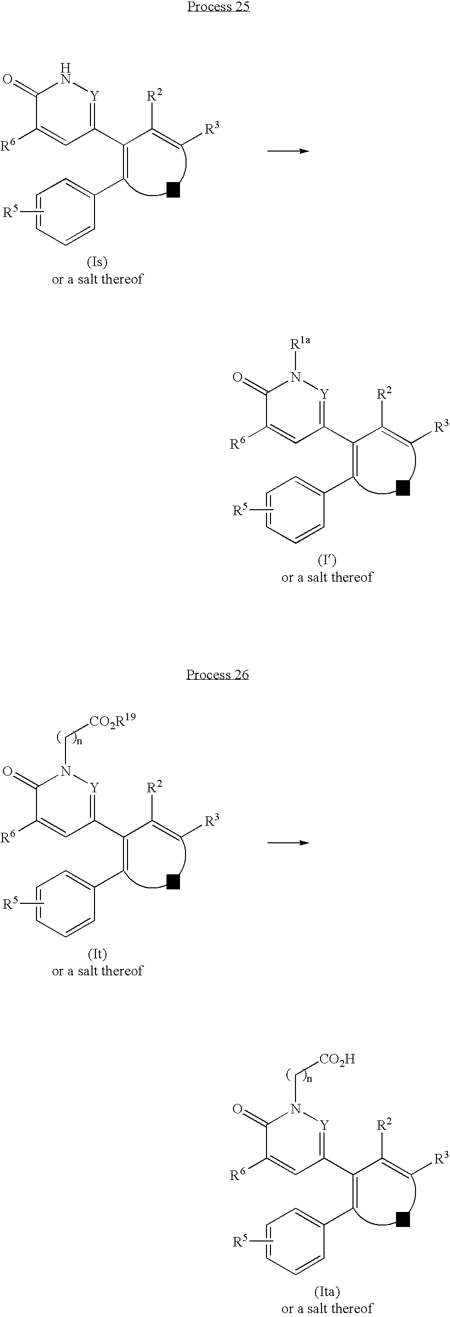

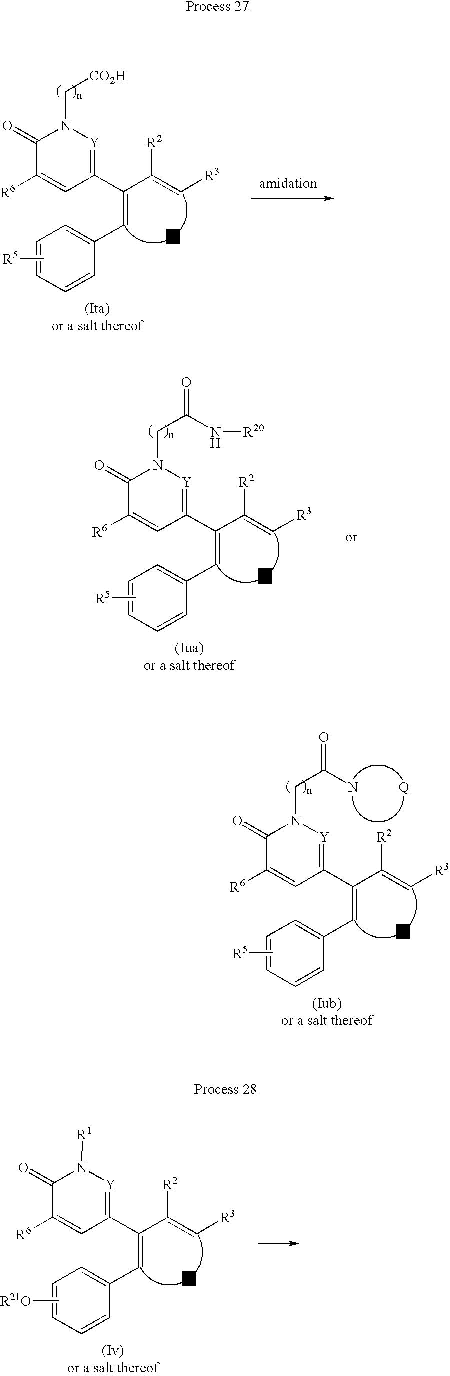

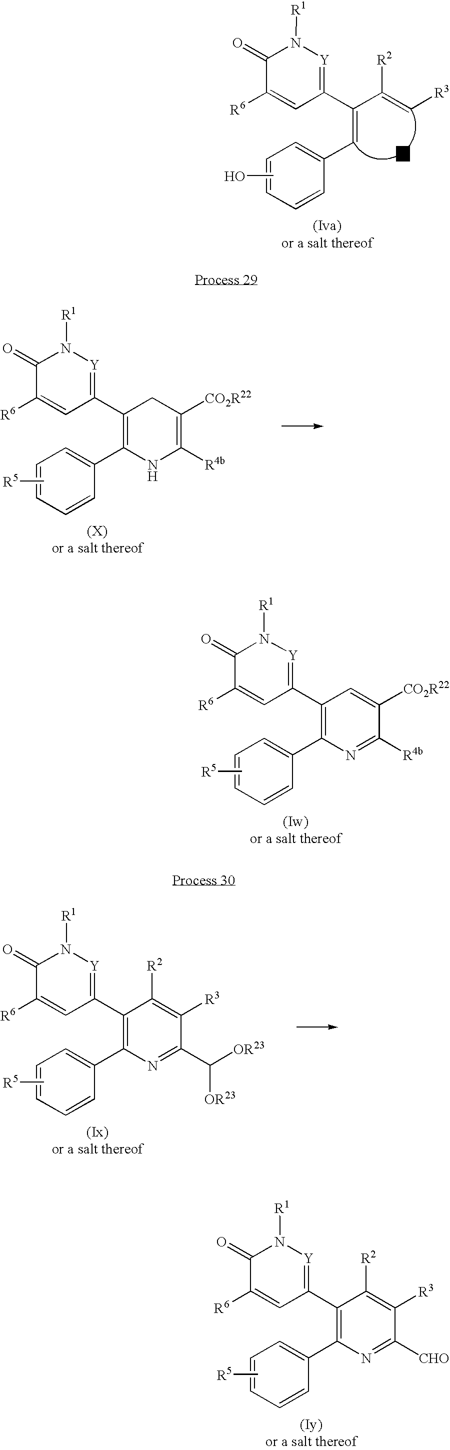

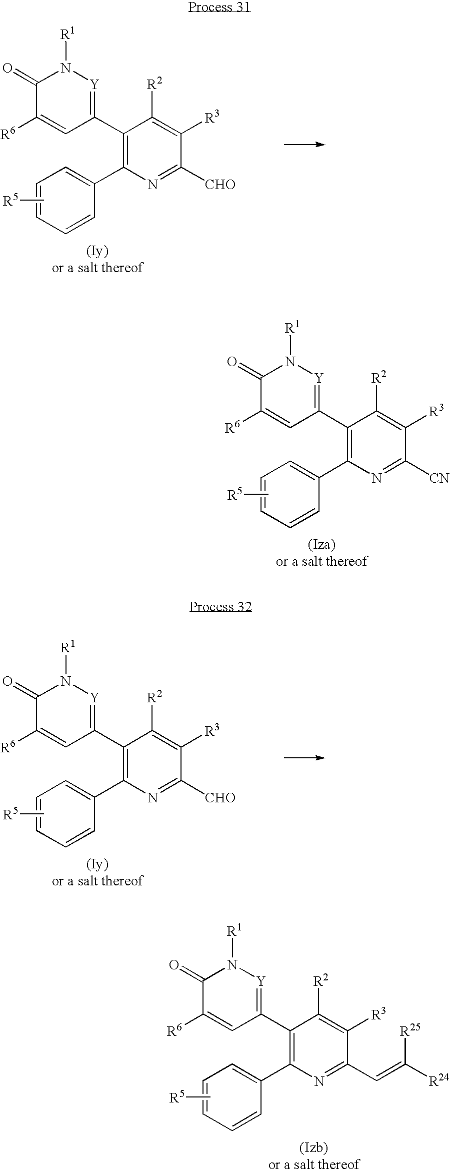

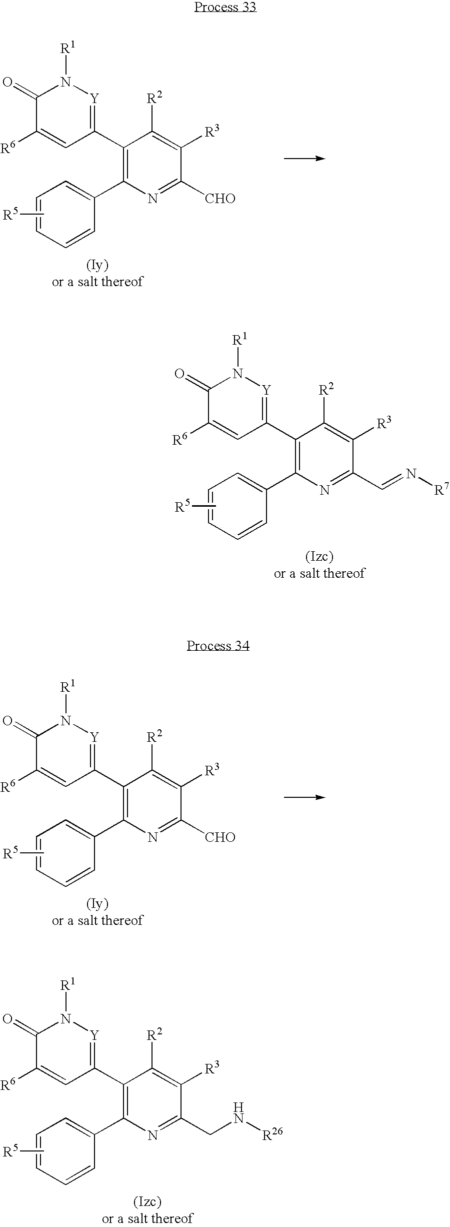

wherein R¹, R², R³, R⁴, R⁶, R⁷, X and Y are as defined above,

R^{1a} is the same as R¹ defined above except for hydrogen,

R^{3a} is the same as R³ defined above except for hydrogen,

R^{4a} is the same as R⁴ defined above except for halogen,

R^{4b}, R¹¹, R¹³, R¹⁶, R¹⁹ and R²⁶ are each optionally substituted lower alkyl,

R⁹, R¹⁰, R¹², R¹⁸, R²¹, R²² and R²³ are each lower alkyl,

R¹⁴ is optionally substituted lower alkyl, aryl or heterocyclic group,

R¹⁵ and R¹⁷ are each optionally substituted aryl or heterocyclic group,

R²⁰ is hydrogen or optionally substituted lower alkyl,

R²⁴ and R²⁵ are each independently hydrogen or the same as R⁷ defined above,

Z is hydrogen or alkali metal,

Hal is a halogen atom,

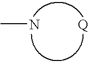

is optionally substituted heteromonocyclic group containing nitrogen atom(s), and n is 1 or 2.

The starting compounds or a salt thereof is novel and can be prepared, for example, by the following reaction schemes.

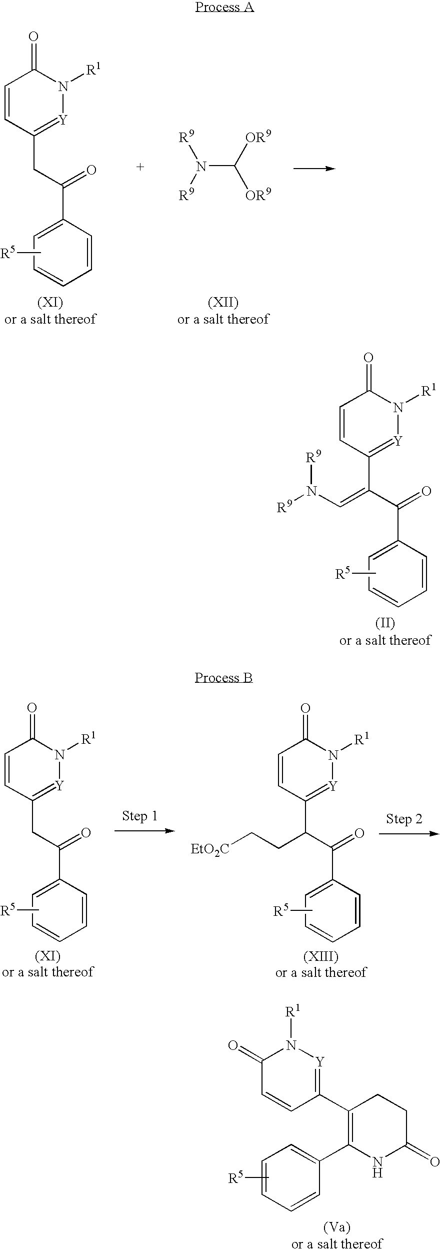

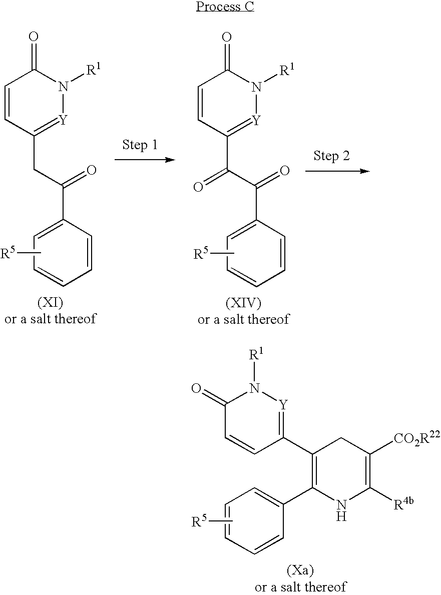

wherein R¹, R^{4b}, R⁵, R⁹, R²² and Y are each as defined above.

In addition to the processes as mentioned above, the object compound (I) and a salt thereof can be prepared, for example, according to the procedures as illustrated in Examples in the present specification or in a manner similar thereto.

The starting compounds can be prepared, for example, according to the procedures as illustrated in Preparations in the present specification or in a manner similar thereto.

The object compound (I) and a salt thereof can be prepared according to the methods as shown in Preparations or Examples, or in a manner similar thereto.

It is to be noted that the object compound (I) may include the geometrical isomer(s) due to the double bond(s) and/or the stereo isomer(s) due to the asymmetric carbon atom(s). In this regard, one isomer can be converted to another according to a conventional method in this field of the art.

It is also to be noted that the solvating form of the compound (I) (e.g. hydrate, etc.) and any form of the crystal of the compound (I) are included within the scope of the present invention.

It is also to be noted that radiolabelled derivatives of compound (I), which are suitable for biological studies, are included within the scope of the present invention.

Suitable salts of the object compound (I) are conventional pharmaceutically acceptable ones and include a metal salt such as an alkali metal salt (e.g. sodium salt, potassium salt, etc.) and an alkaline earth metal salt (e.g. calcium salt, magnesium salt, etc.), an ammonium salt, an organic base salt (e.g. trimethylamine salt, triethylamine salt, pyridine salt, picoline salt, dicyclohexylamine salt, N,N′-dibenzylethylenediamine salt, etc.), an organic acid salt (e.g. acetate, trifluoroacetate, maleate, tartrate, fumarate, methanesulfonate, benzenesulfonate, formate, toluenesulfonate, etc.), an inorganic acid salt (e.g. hydrochloride, hydrobromide, hydriodide, sulfate, phosphate, etc.), a salt with an amino acid (e.g. arginine, aspartic acid, glutamic acid, etc.), and the like.

Suitable examples and illustrations of the various definitions which the present invention includes within the scope thereof and which appear in the above and following description in the present specification are explained in detail as follows.

The term “lower” is intended to mean 1 to 6 carbon atom(s) unless otherwise indicated.

Suitable “lower alkyl” may include straight or branched ones such as methyl, ethyl, propyl, isopropyl, butyl, tert-butyl, pentyl, hexyl or the like, in which the preferred one may be methyl or isopropyl.

Suitable “optionally substituted lower alkyl” may include lower alkyl which is optionally substituted by suitable substituent(s) such as lower alkoxy, hydroxy, cyclo(lower)alkyl, amino, aryl, heterocyclic group, acyl or the like, in which the preferred one may be hydroxymethyl, hydroxyethyl, dimethoxymethyl, aminoethyl, acetoxymethyl, bis(methoxycarbonyl)methyl, benzyl, pyridylmethyl, piperidinylethyl, morpholinylethyl or carbamoylmethyl.

Suitable “lower alkoxy” may include straight or branched ones such as methoxy, ethoxy, propoxy, isopropoxy, butoxy, tert-butoxy, pentyloxy, hexyloxy or the like, in which the preferred one may be (C1-C4)alkoxy and the more preferred one may be methoxy.

Suitable “optionally substituted lower alkoxy” may include lower alkoxy which is optionally substituted by suitable substituent(s) such as hydroxy, cyclo(lower)alkyl, amino, aryl, heterocyclic group, acyl or the like, in which the preferred one may be dimethylaminoethoxy, aminoethoxy, triazolylmethoxy or carbamoylmethoxy.

Suitable “cyclo(lower)alkyl” may be cyclo(C3-C8)alkyl such ascyclopropyl, cyclobutyl, cyclopentyl, cyclohexyl, cycloheptyl, cyclooctyl or the like, in which the preferred one may be cyclohexyl.

Suitable “aryl” may include phenyl, naphthyl, indenyl, anthryl, or the like, in which the preferred one may be (C6-C10) aryl, and the more preferred one may be phenyl.

Suitable “aryl(lower)alkyl” may include phenyl(lower)alkyl (e.g. benzyl, phenethyl, etc.), diphenyl(lower)alkyl (e.g. benzhydryl, etc.), triphenyl(lower)alkyl (e.g. trityl, etc.), naphthyl(lower)alkyl, indenyl(lower)alkyl or anthryl(lower)alkyl and the like, in which the preferred one may be phenyl(lower)alkyl, and the more preferred one may be phenyl(C1-C4)alkyl.

Suitable “optionally substituted aryl” may include aryl which is optionally substituted by suitable substituent(s), preferably 1 to 3 substituent(s), such as lower alkyl, lower alkoxy, hydroxy, halogen, etc. Suitable examples of optionally substituted aryl include lower alkylphenyl, lower alkoxyphenyl and halophenyl, in which more preferred one is fluorophenyl.

Suitable “heterocyclic group” may be saturated or unsaturated monocyclic or polycyclic heterocyclic groups containing at least one heteroatom selected from among oxygen, sulfur and nitrogen.

The particularly preferred example of said heterocyclic group may include unsaturated 3- through 8-membered heteromonocyclic groups containing 1 through 4 nitrogen atom(s), such as pyrrolyl, pyrrolinyl, imidazolyl, pyrazolyl, pyridyl and its N-oxide, pyrimidinyl, pyrazinyl, pyridazinyl, triazolyl (e.g. 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), dihydrotriazinyl (e.g. 4,5-dihydro-1,2,4-triazinyl, 2,5-dihydro-1,2,4-triazinyl, etc.), etc, in which more preferred one is pyrrolyl, pyrazolyl and pyridyl.;

3- through 8-membered saturated heteromonocyclic groups containing 1 through 4 nitrogen atom(s), such as pyrrolidinyl, imidazolidinyl, piperidyl (e.g. piperidino, etc.), piperazinyl, etc, in which more preferred one is piperidyl and piperazinyl.;

unsaturated condensed heterocyclic groups containing 1 through 5 nitrogen atom(s), such as indolyl, isoindolyl, indolizinyl, benzimidazolyl, quinolyl, isoquinolyl, indazolyl, benzotriazolyl, tetrazolopyridyl, tetrazolopyridazinyl (e.g. tetrazolo[1,5-b]pyridazinyl etc.), dihydrotriazolopyridazinyl, etc.;

3- through 8-membered unsaturated heteromonocyclic groups containing 1 or 2 oxygen atoms and 1 through 3 nitrogen atom(s), such as oxazolyl, isoxazolyl, oxadiazolyl (e.g. 1,2,4-oxadiazolyl, 1,3,4-oxadiazolyl, 1,2,5-oxadiazolyl, etc.), etc.;

3- through 8-membered saturated heteromonocyclic groups containing 1 or 2 oxygen atom(s) and 1 through 3 nitrogen atoms, such as morpholinyl, oxazolidinyl (e.g. 1,3-oxazolidinyl etc.), etc.;

unsaturated condensed heterocyclic groups containing 1 or 2 oxygen atom(s) and 1 through 3 nitrogen atom(s), such as benzoxazolyl, benzoxadiazolyl, etc.;

3- through 8-membered unsaturated heteromonocyclic groups containing 1 or 2 sulfur atom(s) and 1 through 3 nitrogen atom(s), such as thiazolyl, isothiazolyl, thiazolinyl, thiadiazolyl (e.g. 1,2,4-thiadiazolyl, 1,3,4-thiadiazolyl, 1,2,5-thiadiazolyl, 1,2,3-thiadiazolyl), etc.;

3- through 8-membered saturated heteromonocyclic groups containing 1 or 2 sulfur atom(s) and 1 through 3 nitrogen atom(s), such as thiazolidinyl etc.;

3- through 8-membered unsaturated heteromonocyclic groups containing 1 sulfur atom, such as thienyl etc.;

unsaturated condensed heterocyclic groups containing 1 or 2 sulfur atoms and 1 through 3 nitrogen atom(s), such as benzothiazolyl, benzothiadiazolyl, etc.;

3- through 8-membered unsaturated heteromonocyclic groups containing 1 or2 oxygen atom(s), such as furyl, pyranyl, dioxolyl, etc.;

3- through 8-membered saturated heteromonocyclic groups containing 1 or 2 oxygen atom(s), such as oxolanyl, tetrahydropyranyl (e.g. tetrahydro-2H-pyran-2-yl etc.), dioxolanyl, etc.; and

unsaturated condensed heterocyclic groups containing 1 or 2 oxygen atom(s), such as isobenzofuranyl, chromenyl (e.g. 2H-chromen-3-yl etc.), dihydrochromenyl (e.g. 3,4-dihydro-2H-chromen-4-yl etc.), etc.

Suitable “N-containing heterocyclic group” may be aforesaid “heterocyclic group”, in which said group contains at least one N atom in its ring members.

Suitable “optionally substituted heterocyclic group” may include heterocyclic group which is optionally substituted by suitable substituent(s), preferably 1 to 3 substituent(s), such as lower alkyl, lower alkoxy, hydroxy, halogen, or the like.

Suitable “acyl” may include lower alkanoyl, carboxy, protected carboxy, and the like.

Suitable examples of aforesaid “lower alkanoyl” may be formyl, acetyl, propionyl, butyryl, isobutyryl, pivaloyl, hexanoyl, or the like, in which the preferred one may be (C1-C4)alkanoyl and the more preferred one may be formyl and acetyl.

Suitable examples of aforesaid “protected carboxy” may be

i) esterified carboxy, in which suitable esterified carboxy may include lower alkoxycarbonyl (e.g. methoxycarbonyl, ethoxycarbonyl, propoxycarbonyl, butoxycarbonyl, t-butoxycarbonyl, pentyloxycarbonyl, hexyloxycarbonyl, etc.), aryl(lower)alkoxycarbonyl (e.g. benzyloxycarbonyl, phenethyloxycarbonyl, 2-phenylpropoxycarbonyl, 4-phenylbutoxycarbonyl, 4-phenylpentyloxycarbonyl, 1,3-diphenylhexyloxycarbonyl, etc.), and the like;

ii) amidated carboxy, in which suitable amidated carboxy may include carbamoyl, N-(lower)alkylcarbamoyl (e.g. N-methylcarbamoyl, N-ethylcarbamoyl, N-isopropylcarbamoyl, N-butylcarbamoyl, N-pentylcarbamoyl, N-hexylcarbamoyl, etc.), N,N-di(lower)alkylcarbamoyl [e.g. N,N-dimethylcarbamoyl, N,N-diethylcarbamoyl, N-methyl-N-ethylcarbamoyl, N,N-dipropylcarbamoyl, N,N-di(t-butyl)carbamoyl, N-pentyl-N-hexylcarbamoyl, etc.], N-lower alkyl-N-ar(lower)alkylcarbamoyl (e.g. N-methyl-N-benzylcarbamoyl, etc), and the like.

Suitable “halogen” may be fluoro, chloro, bromo and iodo.

The processes for preparing the object pyridazinone or pyridone compound (I) are explained in detail in the following.

### Process 1

The compound (Iab) or a salt thereof can be prepared by subjecting the compound (II) or a salt thereof to formation reaction of pyridine ring.

Suitable salt of the compound (II) and (Iab) can be referred to the ones as exemplified for the compound (I).

This reaction can be carried out by reacting the compound (II) or a salt thereof with 2-cyanoacetamide.

The reactions may be carried out in a conventional solvent such as water, alcohol (e.g. methanol, ethanol, etc.), acetone, dioxane, acetonitrile, chloroform, methylene chloride, ethylene dichloride, tetrahydrofuran, ethyl acetate, N,N-dimethylformamide, pyridine or any other organic solvent which does not adversely affect the reaction. These conventional solvents may also be used in a mixture with water. The reaction is preferably conducted in the presence of a base, for example, inorganic base such as alkali metal hydroxide (e.g. sodium hydroxide, potassium hydroxide, etc.), alkali metal carbonate (e.g. sodium carbonate, potassium carbonate, etc.), alkali metal bicarbonate(e.g. sodium hydrogen carbonate, potassium hydrogen carbonate, etc.), alkali metal hydride (e.g. sodium hydride), alkali metal alkoxide (e.g. NaOMe, NaOEt, t-BuOK, etc.) organic base such as trialkylamine, and the like.

The reaction temperature is not critical, and the reaction is usually carried out at ambient temperature, under warming or under heating.

### Process 2

The compound (Ibb) or a salt thereof can be prepared by subjecting the compound (Iaa) or a salt thereof to halogenation.

Suitable salt of the compound (Iaa) can be referred to the ones as exemplified for the compound (I).

The present reaction may be carried out in a conventional manner for transforming oxo group to halogen, by using the compound (III) such as phosphorus oxychloride.

This reaction may be carried out in a conventional solvent which does not adversely influence the reaction such as alcohol [e.g. methanol, ethanol, propanol, etc.], tetrahydrofuran, dioxane, dimethyl sulfoxide, N,N-dimethylformamide, triethylamine hydrochloride or a mixture thereof.

The reaction temperature is not critical, and the reaction is usually carried out at ambient temperature, under warming or under heating.

### Process 3

The compound (Id) or a salt thereof can be prepared by subjecting the compound (Ic) or a salt thereof to hydration reaction.

Suitable salt of the compound (Ic) can be referred to the ones as exemplified for the compound (I).

The present reaction may be carried out in a conventional manner for transforming nitrile to amide.

This reaction may be carried out in a conventional solvent which does not adversely influence the reaction such as water, alcohol [e.g. methanol, ethanol, propanol, etc.], tetrahydrofuran, dioxane, dimethyl sulfoxide, N,N-dimethylformamide, or a mixture thereof.

The reaction temperature is not critical, and the reaction is usually carried out at ambient temperature, under warming or under heating.

### Process 4

The compound (Ic) or a salt thereof can be prepared by subjecting the compound (Id) or a salt thereof to dehydration reaction.

Suitable salt of the compound (Id) can be referred to the ones as exemplified for the compound (I).

The dehydrating agent to be used in this dehydration reaction may include phosphorus oxychloride, thionyl chloride, phosphorus pentoxide, phosphorus pentachloride, phosphorus pentabromide and the like.

The present reaction may be carried out in a solvent such as dioxane, chloroform, methylene chloride, 1,2-dichloroethane, tetrahydrofuran, pyridine, acetonitrile, N,N-dimethylformamide or any other solvent which does not adversely affect the reaction.

The reaction temperature is not critical, and the reaction is usually carried out at ambient temperature, under warming or under heating.

### Process 5

The compound (Ie) or a salt thereof can be prepared by dehalogenation or esterification or reacting the compound (Ib) or a salt thereof with the compound (IV) or a salt thereof.

Suitable salt of the compound (Ib) and (IV) can be referred to the ones as exemplified for the compound (I).

The present reaction may be carried out in a solvent such as water, alcohol [e.g. methanol, ethanol, propanol, etc.], tetrahydrofuran, dioxane, dimethyl sulfoxide, N,N-dimethylformamide, chloroform, methylene chloride, 1,2-dichloromethane, pyridine, acetonitrile, or a mixture thereof or any other solvent which does not adversely affect the reaction.

The reaction temperature is not critical, and the reaction is usually carried out at ambient temperature, under warming or under heating.

The dehalogenation reaction can be carried out by the method disclosed in Example 6, etc. mentioned later or the similar manner thereto.

The esterification reaction can be carried out by the method disclosed in Example 9, etc. mentioned later or the similar manner thereto.

And the reaction with the compound (IV) can be carried out by the method disclosed in Example 3, etc. mentioned later or the similar manner thereto.

### Process 6

The compound (Ifa) or a salt thereof can be prepared by carboxylating the compound (Ic) or a salt thereof.

This reaction can be carried out in a similar manner as in Example 13 mentioned below.

### Process 7

The compound (Ifa) or a salt thereof can be prepared by carboxylating the compound (Id) or a salt thereof.

This reaction can be carried out by the method disclosed in Example 14, etc. mentioned later or the similar manner thereto.

### Process 8

The compound (If) or a salt thereof can be prepared by esterificating the compound (Ifa) or a salt thereof.

This reaction can be carried out by the method disclosed in Example 42, etc. mentioned later or the similar manner thereto.

### Process 9

The compound (Iga) or a salt thereof can be prepared by subjecting the compound (Ig) or a salt thereof to hydrolysis.

The hydrolysis is preferably carried out in the presence of a base or an acid including Lewis acid.

Suitable base includes an inorganic base and organic base such as an alkali metal (e.g. sodium, potassium, etc.), an alkaline earth metal (e.g. magnesium, calcium, etc.), the hydroxide or carbonate or bicarbonate thereof, trialkylamine (e.g. trimethylamine, triethylamine, etc.), hydrazine, picoline, 1,5-diazabicyclo[4.3.0]non-5-ene, 4,4-diazabicyclo[2.2.2]octane, 1,8-diazabicyclo[5.4.0]undec-7-ene, or the like.

Suitable acid includes an organic acid (e.g. formic acid, acetic acid, propionic acid, trichloroacetic acid, trifluoroacetic acid, etc.) and an inorganic acid (e.g. hydrochloric acid, hydrobromic aacid, sulfuric acid, hydrogen chloride, hydrogen bromide, etc.).

The elimination using Lewis acid such as boron tribromide, boron trichloride, boron trifluoride, alminium chloride, titanium trichloride or the like is preferably carried out in the presence of cation trapping agents (e.g. anisole, phenol, etc.).

The reaction is usually carried out in a solvent such as water, an alcohol (e.g. methanol, ethanol, isopropyl alcohol, etc.), tetrahydrofuran, dioxane, toluene, methylene chloride, ethylene dichloride, chloroform, N,N-dimethylformamide, N,N-dimethylacetamide, or any other organic solvent which does not adversely affect the reaction, or a mixture thereof.

A liquid base or acid can be also used as the solvent.

The reaction temperature is not critical and the reaction is usually carried out under cooling to heating.

This reaction can be carried out by the method disclosed in Example 11, etc. mentioned later or the similar manner thereto.

### Process 10

The compound (Iha) or a salt thereof and the compound (Ihb) or a salt thereof can be prepared by amidating the compound (Iga) or a salt thereof.

This reaction can be carried out by the method disclosed in Example 15, etc. mentioned later or the similar manner thereto.

In this reaction, a reactive derivative at the carboxy group may used. Suitable reactive derivative may include an acid halide, an acid azide, an acid anhydride (mixed acid anhydride or symmetrical acid anhydride), an activated amide (e.g. an activated amide with imidazole, triazole, tetrazole, etc.), an activated ester (e.g. cyanomethyl ester, methoxymethyl ester, dimethyliminomethyl ester, trichlorophenyl ester, p-nitrophenyl ester, or an ester with a N-hydroxy compound (e.g. N-hydroxysuccinimide, 1-hydroxy-1H-benzotriazole, etc.), and the like.

The reaction can be carried out in a conventional solvent such as water, alcohol [e.g. methanol, ethanol, etc.], acetone, dioxane, acetonitrile, chloroform, methylene chloride, ethylene chloride, tetrahydrofuran, ethylacetate, N,N-dimethylformamide, pyridine or any other organic solvent which does not adversely influence the reaction. These conventional solvents may also be used in a mixture with water.

This reaction is preferably carried out in the presence of a conventional condensing agent such as N,N′-dicyclohexylcarbodiimide, N,N′-diethylcarbodiimide, N,N′-diisopropylcarbodiimide, 1-ethyl-3-[3′-(dimethylamino)propyl]carbodiimide, N,N′-carbonylbis(2-methylimidazole), diphenylketene-N-cyclohexylimine, ethoxyacetylene, ethyl polyphosphate, isopropyl polyphosphate, phosphorus oxychloride (phosphoryl chloride), phosphorus trichloride, diphenyl phosphorylazide, thionyl chloride, oxalyl chloride, lower alkyl haloformate (e.g. ethyl chloroformate, isopropyl chloroformate, etc.), triphenylphosphine, so-called Vilsmeier reagent prepared by the reaction of N,N-dimethylformamide with thionyl chloride, phosgene, trichloromethylchloroformate, phosphorus oxychloride, etc., or the like.

The reaction may also be carried out in the presence of an inorganic or organic base such as an alkali metal bicarbonate, tri(lower)alkylamine, pyridine, alkali metal hydroxide, or the like.

The reaction temperature is not critical, and the reaction can be carried out under cooling to warming.

### Process 11

The compound (Iia) or a salt thereof can be prepared by subjecting acylamino group for carboxy group of the compound (Ifa) or a salt thereof.

This reaction can be carried out by the method disclosed in Example 25, etc. mentioned later or the similar manner thereto.

### Process 12

The compound (Ii) or a salt thereof can be prepared by hydrolyzing acylamino group of the compound (Iia) or a salt thereof.

This reaction can be carried out by the method disclosed in Example 26, etc. mentioned later or the similar manner thereto.

### Process 13

The compound (Iac) or a salt thereof can be prepared by dehydrogenating the compound (V) or a salt thereof.

This reaction can be carried out by the method disclosed in Example 30, etc. mentioned later or the similar manner thereto.

### Process 14

The compound (Ij) or a salt thereof can be prepared by alkylating oxygen atom of the compound (Ia) or a salt thereof.

The present reaction may be carried out in a solvent such as water, phosphate buffer, acetone, chloroform, acetonitrile, nitrobenzene, methylene chloride, ethylene dichloride, formamide, N,N-dimethylformamide, methanol, ethanol, sec-butanol, amyl alcohol, diethyl ether, dioxane, tetrahydrofuran, dimethyl sulfoxide, or any other organic solvent, which does not adversely affect the reacyion, preferably in ones having strong polarities. Among the solvents, hydrophilic solvents may used in a mixture with water. The reaction is preferably conducted in the presence of base, for example, in organic base such as alkali metal hydroxide, alkalimetal carbonate, alkalimatal bicarbonate, alkali metal hydride (e.g. sodium hydride, etc.), organic base such as trialkylamine, and the like.

The reaction temperature is not critical, and the reaction is usually carried out at ambient temperature, under warming or under heating.

The present reaction is preferably carried out in the presence of alkali metal halide (e.g. sodium iodide, potassium iodide, etc.), alkali metal thiocyanate (e.g. sodium thiocyanate, potassium thiocyanate, etc.), di(lower)alkyl azodicarboxylate (e.g. diethyl azodicarboxylate, diisopropyl azodicarboxylate, etc.) or the like.

This reaction can be carried out by the method disclosed in Example 31, etc. mentioned later or the similar manner thereto.

### Process 15

The compound (Ik) or a salt thereof can be prepared by amidating the compound (Ija) or a salt thereof.

This reaction can be carried out by the method disclosed in Example 32, etc. mentioned later or the similar manner thereto.

### Process 16

The compound (Ila) or a salt thereof can be prepared by amidating the compound (Il) or a salt thereof.

This reaction can be carried out in the same manner as in the aforementioned Process 10, and therefore the reagents to be used and the reaction conditions (e.g., solvent, reaction temperature, etc.) can be referred to those of Process 10.

### Process 17

The compound (In) or a salt thereof can be prepared by reacting the compound (Im) or a salt thereof with the compound (VI) or a salt thereof.

This reaction can be carried out by the method disclosed in Example 33, etc. mentioned later or the similar manner thereto.

### Process 18

The compound (Iba) or a salt thereof can be prepared by reacting the compound (Io) or a salt thereof with the compound (VI) or a salt thereof.

This reaction can be carried out by the method disclosed in Example 75, etc. mentioned later or the similar manner thereto.

### Process 19

The compound (Iq) or a salt thereof can be prepared by reacting the compound (Ip) or a salt thereof with the compound (VII) or a salt thereof.

This reaction can be carried out by the method disclosed in Example 36, etc. mentioned later or the similar manner thereto.

### Process 20

The compound (Im) or a salt thereof can be prepared by subjecting the compound (Ifa) or a salt thereof to decarboxylation.

This reaction can be carried out by the method disclosed in Example 45, etc. mentioned later or the similar manner thereto.

### Process 21

The compound (Ira) or a salt thereof can be prepared by subjecting the compound (Ii) or a salt thereof to hydroxylation.

This reaction can be carried out by the method disclosed in Example 53, etc. mentioned later or the similar manner thereto.

### Process 22

The compound (Ir) or a salt thereof can be prepared by alkylating oxygen atom of the compound (Ira) or a salt thereof.

This reaction can be carried out in the same manner as in the aforementioned Process 14, and therefore the reagents to be used and the reaction conditions (e.g., solvent, reaction temperature, etc.) can be referred to those of Process 14.

### Process 23

The compound (Ilb) or a salt thereof can be prepared by subjecting the compound (Il) to reductive amination with the compound (VIII).

This reaction can be carried out by the method disclosed in Example 67, etc. mentioned later or the similar manner thereto.

In this reaction, a reactive derivative at the amino group may used. Suitable reactive derivative may include Schiff's base type amino or its tautomeric enamine type isomer formed by the reaction of the compound (Il) with a carbonyl compound (e.g. aldehyde, ketone or the like), a silyl derivative formed by the reaction of the compound (Il) with a silyl compound (e.g. bis(trimethylsilyl)acetamide, mono(trimethylsilyl)acetamide, bis(trimethylsilyl)urea or the like), a derivative formed by reaction of the compound (Il) with phosphorus trichloride or phosgene, and the like.

The reaction is usually carried out in a conventional solvent such as water, alcohol (e.g. methanol, ethanol, etc.), acetone, dioxane, acetonitrile, chloroform, methylene chloride, ethylene chloride, tetrahydrofuran, ethyl acetate, N,N-dimethylformamide, pyridine or any other organic solvent which does not adversely influence the reaction, or the mixture thereof.

The reaction may also be carried out in the presence of a reductive regent such as hydrides (e.g. hydrogen iodide, hydrogen sulfide, lithium aluminum hydride, sodium borohydride, sodium cyanoborohydride, sodium triacetoxyborohydride, etc.), or the like.

The reaction temperature is not critical, and the reaction is usually carried out under cooling to heating.

### Process 24

The compound (Is) or a salt thereof can be prepared by subjecting the compound (IX) or a salt thereof to hydrolysis.

This reaction can be carried out in the same manner as in the aforementioned Process 9, and therefore the reagents to be used and the reaction conditions (e.g., solvent, reaction temperature, etc.) can be referred to those of Process 9.

### Process 25

The compound (I′) or a salt thereof can be prepared by alkylating nitrogen atom of the compound (Is) or a salt thereof.

This reaction can be carried out in the same manner as in the aforementioned Process 14, and therefore the reagents to be used and the reaction conditions (e.g., solvent, reaction temperature, etc.) can be referred to those of Process 14.

This reaction can be carried out by the method disclosed in Example 87, etc. mentioned later or the similar manner thereto.

### Process 26

The compound (Ita) or a salt thereof can be prepared by subjecting the compound (It) or a salt thereof to hydrolysis.

This reaction can be carried out in the same manner as in the aforementioned Process 9, and therefore the reagents to be used and the reaction conditions (e.g., solvent, reaction temperature, etc.) can be referred to those of Process 9.

### Process 27

The compound (Iua) or a salt thereof and the compound (Iub) or a salt thereof can be prepared by amidating the compound (Ita) or a salt thereof.

This reaction can be carried out in the same manner as in the aforementioned Process 10, and therefore the reagents to be used and the reaction conditions (e.g., solvent, reaction temperature, etc.) can be referred to those of Process 10.

### Process 28

The compound (Iva) or a salt thereof can be prepared by subjecting the compound (Iv) or a salt thereof to elimination reaction of alkyl group.

This reaction is carried out in accordance with a conventional method such as hydrolysis.

The hydrolysis can be carried out in the same manner as in the aforementioned Process 9, and therefore the reagents to be used and the reaction conditions (e.g., solvent, reaction temperature, etc.) can be referred to those of Process 9.

### Process 29

The compound (Iw) or a salt there of can be prepared by subjecting the compound (X) or a salt thereof to dehydrogenation.

This reaction is carried out in accordance with a conventional method such as oxidation.

The oxidation can be carried out in the presence of catalyst such as manganese(IV) oxide.

The present reaction may be carried out in a solvent such as dioxane, chloroform, methylene chloride, 1,2-dichloroethane, tetrahydrofuran, pyridine, acetonitrile, N,N-dimethylformamide, N,N-dimethylacetamide or any other solvent which does not adversely affect the reaction.

The reaction temperature is not critical, and the reaction is usually carried out at ambient temperature, under warming or under heating.

### Process 30

The compound (Iy) or a salt thereof can be prepared by subjecting the compound (Ix) or a salt thereof to hydrolysis.

The hydrolysis can be carried out in the same manner as in the aforementioned Process 9, and therefore the reagents to be used and the reaction conditions (e.g., solvent, reaction temperature, etc.) can be referred to those of Process 9.

### Process 31

The compound (Iza) or a salt thereof can be prepared by reacting the compound (Iy) or a salt thereof with hydroxylamine in the presence of sodium acetate, following to hydrolysis.

This reaction can be carried out by the method disclosed in Example 134, etc. mentioned later or the similar manners thereto.

### Process 32

The compound (Izb) or a salt thereof can be prepared by subjecting the compound (Iy) or a salt thereof to olefin formation reaction.

This reaction is carried out in accordance with a conventional method such as Horner-Wadsworth-Emmons reaction, Wittig reaction, or the like.

This reaction can be carried out by the method disclosed in Example 138 or 150, etc. mentioned later or the similar manners thereto.

### Process 33

The compound (Izc) or a salt thereof can be prepared by reacting the compound (Iy) or a salt thereof with N-optionally substituted hydroxylamine.

This reaction can be carried out by the method disclosed in Example 136 or 155, etc. mentioned later or the similar manners thereto.

### Process 34

The compound (Izd) or a salt thereof can be prepared by subjecting the compound (Iy) or a salt thereof to reductive amination.

The hydrolysis can be carried out in the same manner as in the aforementioned Process 23, and therefore the reagents to be used and the reaction conditions (e.g., solvent, reaction temperature, etc.) can be referred to those of Process 23.

### Process A

The compound (II) or a salt thereof can be prepared by reacting the compound (XI) or a salt thereof with the compound (XII) or a salt thereof.

This reaction can be carried out by the method disclosed in Preparation 1, etc. mentioned later or the similar manner thereto.

### Process B

The reaction of Step 1 can be carried out by the method disclosed in Preparation 2, etc. mentioned later or the similar manners thereto. The reaction of Step 2 can be respectively carried out by the method disclosed in Preparation 3, etc. mentioned later or the similar manners thereto.

### Process C

The reaction of Step 1 can be carried out by the method disclosed in Preparation 18, etc. mentioned later or the similar manners thereto. The reaction of Step 2 can be respectively carried out by the method disclosed in Preparation 19, etc. mentioned later or the similar manners thereto.

The object compound (I) of the present invention is an adenosine antagonist and possesses the various pharmacological actions as stated before.

In order to show the usefulness of the compound (I) of the present invention, the pharmacological test result of the representative compound of the present invention is shown in the following.

### Test 1 : Adenosine Antagonistic Activity

[I] Test Method

The adenosine antagonistic activity [Ki(nM)] of the test compound was examined by radioligand binding techniques using 8-cyclopentyl-1,3-dipropylxanthine, [dipropyl-2,3-³H (N)] ([³H]DPCPX, 4.5 nM) for human A₁ receptor and [³H]CGS 21680 (20 nM) for human A₂ₐ receptor.

[II] Test Compound

2-Amino-5-(1-isopropyl-6-oxo-1,6-dihydro-3-pyridazinyl)-6-phenylnicotinonitrile (Example 3)

2-isopropyl-6-[2-phenyl-5-(pyrazol-5-yl)-3-pyridyl]-3(2H)-pyridazinone (Example 24)

N-[5-(1-isopropyl-6-oxo-1,6-dihydro-3-pyridazinyl)-6-phenyl-3-pyridyl]benzamide (Example 27)

6-(6-amino-5-bromo-2-phenyl-3-pyridyl)-2-isopropyl-3(2H)-pyridazinone (Example 33)

6-[6-Amino-5-(4-phenyl-1,3-thiazol-2-yl)-2-phenyl-3-pyridyl]-2-isopropyl-3(2H)-pyridazinone (Example 40)

6-(5-hydroxy-2-phenyl-3-pyridyl)-2-isopropyl-3(2H)-pyridazinone (Example 53)

6-[6-Amino-5-chloro-2-(4-fluorophenyl)-3-pyridyl]-2-isopropyl-3(2H)-pyridazinone (Example 74)

6′-Amino-5,5′-dichloro-1-isopropyl-2′-phenyl-3,3′-bipyridin-6(1H)-one (Example 119)

[III] Test Result

TABLE 1

Adenosine
receptor binding
(Ki:nM)

| Test compound (Example No.) | A₁ | A₂ₐ |
|---|---|---|
| 3 | 0.56 | 0.65 |
| 24 | 0.46 | 2.70 |
| 27 | 0.31 | 1.24 |
| 33 | 0.41 | 0.91 |
| 40 | 4.44 | 6.58 |
| 53 | 0.43 | 3.87 |
| 74 | 6.42 | 1.28 |
| 119 | 1.99 | 2.06 |

### Test 2

### Anticatalepsy Activity in Mouse

[I] Test Method

The test compound (3.2 mg/kg) was administered orally with ddY mice (n=7). Then, haloperidol (0.32 mg/kg) was injected intraperitoneally 30 min. after the administration of the compound. Thirty min. After the injection, the cataleptic responses of mice were measured. The forelimbs of each mouse were placed on a 3 cm high, 3 mm wide horizontal bar, and the duration of cataleptic posture was measured for up to 30 sec.

[II] Test Compound

2-Amino-5-(1-isopropyl-6-oxo-1,6-dihydro-3-pyridazinyl)-6-phenylnicotinonitrile (Example 3)

2-isopropyl-6-[2-phenyl-5-(pyrazol-5-yl)-3-pyridyl]-3(2H)-pyridazinone (Example 24)

N-[5-(1-isopropyl-6-oxo-1,6-dihydro-3-pyridazinyl)-6-phenyl-3-pyridyl]benzamide (Example 27)

6-[6-Amino-5-chloro-2-(4-fluorophenyl)-3-pyridyl]-2-isopropyl-3(2H)-pyridazinone (Example 74)

6′-Amino-5,5′-dichloro-1-isopropyl-2′-phenyl-3,3′-bipyridin-6(1H)-one (Example 119)

[III] Test Result

TABLE 2

| Test compound | Manifestation rate of catalepsy |
|---|---|
| (Example No.) | (number of mouse) |
| 3 | 0/7 |
| 24 | 0/7 |
| 27 | 0/7 |
| 74 | 0/7 |
| 119 | 0/7 |

The pyridazinone or pyridone compound (I) and a salt thereof of this invention are useful as adenosine antagonists (especially, A₁ receptor and A₂ (particularly A₂ₐ) receptor dual antagonists) and for the prevention and/or the treatment of depression, dementia (e.g. Alzheimer's disease, cerebrovascular dementia, dementia accompanying Parkinson's disease, etc.), Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's ndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack, angina pectoris, and the like.

Adenosine antagonists can be useful for Parkinson's disease by co-administrating with L-3,4-dihidroxy-phenylalanine (L-DOPA), which is the most popular drug for Parkinson's disease (R. Grondin et. al, Neurology, 52, 1673-1677(1999)). So the combination use of the pyridazinone or pyridone compound (I) and a salt thereof of this invention with L-DOPA may be also useful for treatment and/or prevention of Parkinson's disease with decreasing or reducing the side effect such as the onset of dyskinesia eliciting by the long-team application of L-DOPA, and so on.

And additionally, as to a series of the compounds disclosed in our previous patents and patent applications of this field (e.g. WO 99/24424, WO 02/18382, WO 02/100864, WO 03/039451, WO 03/057689, etc.), the combination use with L-DOPA may be also useful same as mentioned above.

Further, in view of the field using these compounds for as a medicament, these compounds should be durable to some degree. And the duration time of the pyridazinone or pyridone compound (I) or a salt thereof of this invention are expected to be long-lasting.

The pharmaceutical composition of this invention can be used in the form of a pharmaceutical preparation, for example, in a solid, semisolid or liquid form, which contains the pyridazinone or pyridone compound (I) or a pharmaceutically acceptable salt thereof as an active ingredient in admixture with an organic or inorganic carrier or excipient suitable for rectal, pulmonary (nasal or buccal inhalation), nasal, ocular, external (topical), oral or parenteral (including subcutaneous, intravenous and intramuscular) administrations or insufflation. The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. In addition, auxiliary, stabilizing agents, thickening agents, coloring agents and perfumes maybe used where necessary. The pyridazinone or pyridone compound (I) or a pharmaceutically acceptable salt thereof is included in a pharmaceutical composition in an amount sufficient to produce the desired aforesaid pharmaceutical effect upon the process or condition of diseases.

For applying the composition to a human being or an animal, it is preferable to apply it by intravenous, intramuscular, pulmonary or oral administration, or insufflation. While the dosage of therapeutically effective amount of the pyridazinone or pyridone compound (I) varies depending on the age and condition of each individual patient to be treated, in the case of intravenous administration, a daily dose of 0.01-100 mg of the pyridazinone or pyridone compound (I) per kg weight of a human being or an animal, in the case of intramuscular administration, a daily dose of 0.1-100 mg of the pyridazinone or pyridone compound (I) per kg weight of a human being or an animal, and in case of oral administration, a daily dose of 0.5-100 mg of the pyridazinone or pyridone compound (I) per kg weight of a human being or an animal is generally given for the prevention and/or treatment of the aforesaid diseases.

The following Preparations and Examples are given for the purpose of illustrating the present invention in more detail.

The abbreviations, symbols and terms used in the Preparations and Examples have the following meanings.

| | |
|---|---|
| AcOH: | acetic acid |
| CH₂Cl₂: | dichloromethane |
| CHCl₃: | chlorofarm |
| DME: | 1,2-dimethoxyethane |
| DMF: | N,N-dimethylformamide |
| DMSO: | dimethyl sulfoxide |
| Et₃N: | triethylamine |
| EtOAc: | ethyl acetate |
| EtOH: | ethanol |
| IPE: | diisopropyl ether |
| MeOH: | methanol |
| THF: | tetrahydrofuran |
| HCl | hydrochloric acid |
| H₂O₂: | hydrogen peroxide |
| H₂SO₄: | sulfuric acid |
| EDCI: | 1-ethyl- |
| | 3-[3′-(dimethylamino)propyl] carbodiimide |
| HOBT: | 1-hydroxybenzotriazole |
| K₂CO₃: | potassium carbonate |
| KOH: | potassium hydroxide |
| MgSO₄: | magnesium sulfate |
| NaBH(OAc)₃: | sodium triacetoxyborohydride |
| NaH: | sodium hydride |
| NaHCO₃: | sodium hydrogen carbonate |
| Na₂CO₃: | sodium carbonate |
| Na₂SO₄: | sodium sulfate |
| NaOAc: | sodium acetate |
| NaOH: | sodium hydroxide |
| NaOMe: | sodium methoxide |
| LiBH₄: | lithium borohydride |
| Pd/C: | palladium on carbon |
| CO: | carbon monoxide |
| aq.: | aqueous |

### Preparation 1

To a mixture of 2-isopropyl-6-(2-oxo-2-phenylethyl)-3(2H)-pyridazinone (500 mg) and N,N-dimethylformamide-dimethoxyacetal (0.518 ml) was heated at 100-105° C. for 1 hour. The mixture was concentrated under reduced pressure to give a residue. The residue was purified by column chromatography on silica gel (CHCl₃) to give 6-[1-benzoyl-2-(dimethylamino)ethenyl]-2-isopropyl-3(2H)-pyridazinone (604 mg) as a solid.

mp: 103-104.5° C. (IPE) IR (KBr): 1647, 1628, 1583, 1554 cm⁻¹; ¹H NMR (CDCl₃, δ): 1.32 (6H, d, J=6.64 Hz), 2.89 (6H, s), 5.33 (1H, 7-plet, J=6.64 Hz), 6.75 (1H, d, J=9.43 Hz), 7.11 (1H, d, J=9.43 Hz), 7.26-7.48 (6H, m); ESI/MS: 645[2M+Na]⁺, 334[M+Na]⁺, 312[M+H]⁺; Elemental Analysis for C₁₈H₂₁N₃O₂.0.1H₂O; Calcd.: C, 69.03; H, 6.82; N, 13.42; Found : C, 69.08; H, 6.75; N, 13.34.

### Preparation 2

A solution of 2-isopropyl-6-(2-oxo-2-phenylethyl)-3(2H)-pyridazinone (200 g) in DMSO (1000 ml) was stirred at 10° C. NaH (32.8 g) was added to the solution. After 30 minutes, the reaction mixture was stirred at ambient temperature for 1 hour. The reaction mixture was cooled at 10° C., 3-bromopropionic acid ethyl ester (105 ml) was added to the reaction mixture under the same conditions. After 4 hours, 1N HCl, water and EtOAc were added to the reaction muxture. The organic layer was separated, and washed with water, aq.NaHCO₃ solution and brine respectively, and dried over MgSO₄. The solvent was removed in vacuo. The residue was purified by silica gel column chromatography eluted with a mixture of n-hexane and EtOAc (3:1). The fractions were concentrated in vacuo to obtain ethyl 4-(1-isopropyl-6-oxo-1,6-dihydro-3-pyridazinyl)-5-oxo-5-phenylpentanoate (198.2 g) as pale yellow oil.

IR (KBr): 3451, 1700, 1662, 1589 cm⁻¹; ¹H NMR (DMSO-d₆, δ): 1.1-1.4 (9H, m), 2.0-2.55 (4H, s), 4.13 (2H, q, J=7.1 Hz), 4.81 (1H, m), 5.27 (1H, 7-plet, J=6.6 Hz), 6.85 (1H, d, J=9.6 Hz), 7.22 (1H, d, J=9.6 Hz), 7.35-7.48 (6H, m), 7.95-8.1 (1H, m); API-ES/MS: 379[M+Na]⁺.

### Preparation 3

A mixture of ethyl 4-(1-isopropyl-6-oxo-1,6-dihydro-3-pyridazinyl)-5-oxo-5-phenylpentanoate (225 g) and ammonium acetate (146 g) in ACOH (450 ml) was stirred at 95° C. After 12 hours, ammonium acetate (100 g) was added to the reaction mixture. After 3 days, the reaction mixture was cooled to 25° C. The solvent was removed in vacuo. Water and EtOAc were added to the reaction mixture. The organic layer was separated, and washed with water, aq.NaHCO₃ solution and brine respectively, and dried over MgSO₄. The solvent was removed in vacuo. The precipitate was collected by filtration to obtain 2-isopropyl-6-(6-oxo-2-phenyl-1,4,5,6-tetrahydro-3-pyridyl)-3(2H)-pyridazinone (135 g) as pale yellow powder.

mp: 88-95° C.; ¹H NMR (DMSO-d₆, δ): 1.09 (6H, d, J=6.6 Hz), 2.4-2.6 (2H, s), 2.7-2.85 (2H, m), 5.01 (1H, 7-plet, J=6.6 Hz), 6.85 (1H, d, J=9.6 Hz), 6.64 (1H, d, J=9.6 Hz), 7.1-7.4 (5H, m), 9.58 (1H, br); API-ES/MS: 310[M+H]⁺, 332[M+Na]⁺.

### Preparation 4

Ethyl 5-(4-fluorophenyl)-4-(1-isopropyl-6-oxo-1,6-dihydro-3-pyridazinyl)-5-oxopentanoate was obtained according to a similar manner to that of Preparation 2.

¹H NMR (CDCl₃, δ): 1.1-1.4 (9H, m), 2.0-2.6 (4H, m), 4.0-4.2 (2H, m), 4.7-4.9 (1H, m), 5.26 (1H, 7-plet, J=6.6 Hz), 6.86 (1H, d, J=9.6 Hz), 7.0-7.3 (3H, m), 7.9-8.2 (1H, m); API-ES/MS: 375[M+1]⁺, 379[M+Na]⁺.

### Preparation 5

6-[2-(4-Fluorophenyl)-6-oxo-1,4,5,6-tetrahydro-3-pyridyl]-2-isopropyl-3(2H)-pyridazinone was obtained according to a similar manner to that of Preparation 3.

¹H NMR (DMSO-d₆, δ): 1.07 (6H, d, J=6.6 Hz), 2.4-2.9 (4H, m), 5.01 (1H, 7-plet, J=6.6 Hz), 6.60 (1H, d, J=9.7 Hz), 6.71 (1H, d, J=9.7 Hz), 7.1-7.4 (4H, m), 9.60 (1H, br); API-ES/MS: 328[M+1]⁺, 350[M+Na]⁺.

### Preparation 6

6-[(E)-1-Benzoyl-2-(dimethylamino)vinyl]-2-methyl-3(2H)-pyridazinone was obtained according to a similar manner to that of Preparation 1.

### Preparation 7

A mixture of 6-ethynyl-2-isopropyl-3(2H) -pyridazinone (27.0 g), bistriphenylphophine palladium dichloride (467 mg), cupper iodide (127 mg), 2-bromo-1-iodobenzene (822.9 ml) and Et₃N (24 ml) in THF (120 ml) was stirred at 70° C. After 4 hours, water, aq.NaHCO₃ solution and EtOAc were added to the reaction mixture at 25° C. The organic layer was separated, washed with water, dried over Na₂SO₄. The solvent was removed in vacuo. The residue was purified by silica gel column chromatography eluted with a mixture of n-hexane and EtOAc(1:1). The fractions were concentrated in vacuo to obtain 6-[(2-bromophenyl)ethynyl]-2-isopropyl-3(2H)-pyridazinone (30.8 g) as pale yellow amorphous powder.

¹H NMR (CDCl₃, δ): 1.41 (6H, d, J=6.6 Hz), 5.33 (1H, 7-plet, J=6.6 Hz), 6.66 (1H, d, J=9.5 Hz), 7.1-7.45 (3H, m), 7.6-7.8 (2H, m); API-ES/MS: 317[M]⁺, 339[M+Na]⁺, 341[M+2+Na]⁺.

### Preparation 8

A mixture of 6-[(2-bromophenyl)ethynyl]-2-isopropyl-3 (2H)-pyridazinone (30.0g) and H₂SO₄ (60 ml) in AcOH (150 ml) was stirred at 100° C. After 1 hour, the reaction mixture was poured into the mixture of ice (900 g) and Na₂CO₃ (180 g) at 25° C. The aqueous solution was extracted with EtOAc. The organic layer was separated, washed with water, dried over Na₂SO₄. The solvent was removed invacuo to obtain 6-[2-(2-bromophenyl)-2-oxoethyl]-2-isopropyl-3 (2H)-pyridazinone (24 g) as pale yellow amorphous powder.

¹H NMR (CDCl₃, δ): 1.29 (6H, d, J=6.6 Hz), 4.28 (2H, s), 5.27 (1H, 7-plet, J=6.6 Hz), 6.88 (1H, d, J=9.5 Hz), 7.21 (1H, d, J=9.5 Hz), 7.25-7.7 (4H, m); API-ES/MS: 337[M+2]⁺, 357[M+Na]⁺, 359[M+2+Na]⁺.

### Preparation 9

A mixture of 6-[2-(2-bromophenyl)-2-oxoethyl]-2-isopropyl-3 (2H)-pyridazinone (28.4 g) and NaH (3.56 g) in DMSO (150 ml) was stirred at 25° C. After 1 hour, 3-bromopropionic acid ethyl ester (810.9 ml) was added into the reaction mixture. After 5 hours, ammonium acetate (39.2 g) was added to the reaction mixture, and stirred at 100° C. for 12 hours. Water was poured into the reaction mixture at 25° C. The aqueous solution was extracted with EtOAc. The organic layer was washed with brine, dried over Na₂SO₄. The solvent was removed in vacuo to give a pale wellow residue. The residue was purified by silica gel column chromatography eluted with a mixture of CHCl₃ and MeOH. The fractions were concentrated in vacuo to afford a yellow powder. The powder was collected by filtration to give 6-[2-(2-bromophenyl)-6-oxo-1,4,5,6-tetrahydro-3-pyridyl]-2-isopropyl-3 (2H)-pyridazinone (20.0 g) as pale yellow powder.

¹H NMR (CDCl₃, δ): 1.1-1.4 (6H, m), 2.4-3.6 (4H, m), 5.0-5.4 (1H, m), 6.8-7.7 (6H, m); API-ES/MS: 410[M+Na]⁺, 412[M+2+Na]⁺.

### Preparation 10

5-(6-Methoxy-3-pyridazinyl)-6-phenyl-3,4-dihydro-2 (1H)-pyridinone was obtained according to a similar manner to that of Preparation 9.

¹H NMR (CDCl₃, δ): 2.6-2.85 (2H, m), 3.0-3.2 (2H, m), 4.10 (3H, s), 6.51 (1H, d, J=9.4 Hz), 6.60 (1H, d, J=9.4 Hz), 7.0-7.5 (6H, m); API-ES/MS: 282[M+H]⁺, 304[M+Na]⁺.

### Preparation 11

5-(6-Methoxy-3-pyridazinyl)-6-phenyl-2 (1H)-pyridinone was obtained according to a similar manner to that of Example 30 mentioned later.

IR (KBr): 3453, 1648 cm⁻¹; ¹H NMR (CDCl₃, δ): 4.00 (3H, s), 6.51 (1H, d, J=9.4 Hz), 6.84 (1H, d, J=9.1 Hz), 6.94 (1H, d, J=9.1 Hz), 7.0-7.5 (5H, m), 7.80 (1H, d, J=9.4 Hz), 11.9 (1H, br); API-ES/MS: 280 [M+H]⁺, 302 [M+Na]⁺.

### Preparation 12

3-Chloro-5-(6-methoxy-3-pyridazinyl)-6-phenyl-2 (1H)-pyridinone was obtained according to a similar manner to that of Example 34 mentioned later.

IR (KBr): 3428, 1648 cm⁻¹; ¹H NMR (CDCl₃, δ): 4.05 (3H, s), 6.88 (1H, d, J=9.2 Hz), 6.97 (1H, d, J=9.2 Hz), 7.1-7.5 (5H, m), 8.06 (1H, s), 12.5 (1H, br); API-ES/MS: 336[M+Na]⁺, 338[M+2+Na]⁺.

### Preparation 13

3-Chloro-5-(6-methoxy-3-pyridazinyl)-6-phenyl-2-pyridinamine was obtained according to a similar manner to that of Example 81 mentioned later.

IR (KBr): 3156, 1641 cm⁻¹; ¹H NMR (CDCl₃, δ): 4.01 (3H, s), 6.73 (2H, br), 6.85-7.05 (2H, m), 7.1-7.4 (5H, m), 7.85 (1H, s), API-ES/MS: 335[M+Na]⁺, 337[M+2+Na]⁺.

### Preparation 14

1′-Isopropyl-2-phenyl-4,5-dihydro-3,3′-bipyridine-6,6′(1H,1′H)-dione was obtained according to a similar manner to that of Preparation 9.

¹H NMR (DMSO-d₆, δ): 0.96 (6H, d, J=6.6 Hz), 2.6-2.8 (4H, m), 5.07 (1H, 7-plet, J=6.6 Hz), 6.47 (1H, d, J=9.6 Hz), 6.75 (1H, d, J=2.5 Hz), 6.9-7.4 (7H, m); API-ES/MS: 309[M+H]⁺, 331[M+Na]⁺.

### Preparation 15

2-Isopropyl-6-[2-(4-methoxyphenyl)-6-oxo-1,4,5,6-tetrahydro-3-pyridyl]-3(2H)-pyridazinone was obtained according to a similar manner to that of Preparation 9.

¹H NMR (DMSO-d₆, δ): 1.16 (6H, d, J=6.6 Hz), 2.4-2.6 (2H, m), 2.65-285 (2H, m), 3.78 (3H, s), 6.4-6.65 (2H, m), 6.8-7.0 (2H, m), 7.1-7.2 (2H, m), 9.52 (1H, br); API-ES/MS: 340[M+H]⁺, 362[M+Na]⁺.

### Preparation 16

2-Isopropyl-6-[2-(2-methoxyphenyl)-6-oxo-1,4,5,6-tetrahydro-3-pyridyl]-3(2H)-pyridazinone was obtained according to a similar manner to that of Preparation 9.

¹H NMR (DMSO-d₆, δ): 1.07 (6H, d, J=6.6 Hz), 2.4-2.6 (2H, m), 2.7-2.9 (2H, m), 3.75 (3H, s), 4.99 (1H, 7-plet, J=6.6 Hz), 6.54 (1H, d, J=9.7 Hz), 6.68 (1H, d, J=9.7 Hz), 6.8-7.1 (3H, m), 7.2-7.4 (2H, m), 7.75 (1H, m), 9.37 (1H, br); API-ES/MS: 340[M+H]⁺, 262[M+Na]⁺.

### Preparation 17

2-Isopropyl-6-[2-(3-methoxyphenyl)-6-oxo-1,4,5,6-tetrahydro-3-pyridyl]-3(2H)-pyridazinone was obtained according to a similar manner to that of Preparation 9.

¹H NMR (CDCl₃, δ): 1.1-1.4 (8H, m), 2.0-2.6 (2H, m), 4.0-4.2 (2H, m), 4.7-4.9 (2H, m), 5.1-5.4 (1H, s), 6.85 (1H, d, J=9.6 Hz), 7.0-7.7 (5H, m), API-ES/MS: 387[M+H]⁺, 409[M+Na]⁺.

### Preparation 18

A mixture of 2-isopropyl-6-(2-oxo-2-phenylethyl)-3 (2H)-pyridazinone (2.56 g), dimethylamine hydrochloride (0.90 g) and paraformaldehyde (0.34 g) in EtOH (50 ml) was refluxed for 2 hours. An additional paraformaldehyde (0.35 g) was added to the mixture, which was refluxed for 2.5 hours further. This procedures were repeated three times. The reaction mixture was evaporated in vacuo and dissolved in EtOAc. The resultant mixture was washed with water, aq.NaHCO₃ solution and water successively. After drying over MgSO₄, the solvent was removed in vacuo to afford a yellow oil, which was subjected to column chromatography on silica gel eluting with CHCl₃. The fractions containing the desired product were combined and evaporated in vacuo to give 2-(2-isopropyl-3 (2H)-pyridazinon-6-yl)-1-phenyl-2-propen-1-one (2.54 g) as an oil.

¹H NMR (DMSO-d₆, δ): 0.84 (6H, d, J=6.59 Hz), 4.95 (1H, 7-plet, J=6.59 Hz), 5.85 (1H, s), 6.36 (1H, s), 6.99 (1H, d, J=9.69 Hz), 7.45-7.79 (5H, m), 7.95 (1H, d, J=9.69 Hz); API-ES/MS: 291[M+Na]⁺.

### Preparation 19

A mixture of 2-(2-isopropyl-3 (2H)-pyridazinon-6-yl)-1-phenyl-2-propen-1-one (0.54 g) and methyl 3-amino-4,4-dimethoxycrotonate (0.39 g) was heated in neat at 110° C. for 10 hours. The reaction mixture was dissolved in EtOAc, washed with water three times and dried over MgSO₄. The solvent was removed in vacuo to give a red oil, which was subjected to column chromatography on silica gel eluting with a mixture of CHCl₃ and EtOAc (40:1). The fractions containing the desired product only were combined and evaporated in vacuo to give methyl 2-dimethoxymethyl-5-(2-isopropyl-3 (2H)-pyrodazinon-6-yl)-6-phenyl-1,4-dihydro-pyridine-3-carboxylate (38.5 mg). The fractions cotaining a mixture of the desired product and the oxidized pyridine derivative (methyl 2-(dimethoxymethyl)-5-(1-isopropyl-6-oxo-1,6-dihydro-3-pyridazinyl)-6-phenylnicotinate) were combined and evaporated to afford a light yellow oil (158.5 mg, when calculated as the desired product).

¹H NMR (DMSO-d₆, δ): 1.12 (6H, d, J=6.60 Hz), 3.39 (6H, s), 3.67 (3H, s), 5.03 (1H, 7-plet, J=6.60 Hz), 5.92 (1H, s), 6.51 (1H, d, J=9.70 Hz), 6.63 (1H, d, J=9.70 Hz), 7.18-7.42 (6H, m); API-ES/MS: 426[M+H]⁺, 448[M+Na]⁺.

### EXAMPLE 1

A mixture of 6-[(E)-1-benzoyl-2-(dimethylamino)ethenyl]-2-isopropyl-3 (2H)-pyridazinone (19.67 g), 28% NaOMe in MeOH solution (26.9 ml) and 2-cyanoacetamide (5.85 g) in DMF (83 ml) was stirred at 80° C. for 2 hours. Water (400 ml) was added to the reaction mixture at ambient temperature to appear brown powder. The precipitate was collected by filtration. The pale brown powder was recrystalized in EtOH to give white powder. The powder was collected by filtration to afford 5-(1-isopropyl-6-oxo-1,6-dihydro-3-pyridazinyl)-2-oxo-6-phenyl-1,2-dihydro-3-pyridine-carbonitrile (18.6 g).

mp: >250° C.; IR (KBr): 2225, 1683, 1664, 1641 cm⁻¹; ¹H NMR (DMSO-d₆, δ): 0.96 (6H, d, J=6.6 Hz), 4.96 (1H, 7-plet, J=6.6 Hz), 6.79 (1H, d, J=9.6 Hz), 7.15 (1H, d, J=9.6 Hz), 7.25-7.35 (2H, m), 7.4-7.5 (3H, m), 8.42 (1H, s), 12.96 (1H, br), APCI/MS: 355[M+Na]⁺; Elemental Analysis for C₁₉H₁₆N₄O₂; Calcd.: C, 68.60; H, 4.85; N, 16.86; Found : C, 68.60; H, 4.89; N, 16.81.

### EXAMPLE 2

A mixture of 5-(1-isopropyl-6-oxo-1,6-dihydro-3-pyridazinyl)-2-oxo-6-phenyl-1,2-dihydro-3-pyridine-carbonitrile (200 mg), phosphorus oxychloride (337 μl) and triethylamine hydrochloride (99 mg) was stirred at 110° C. for 1.5 hours. Water (4.0 ml) was added to the reaction mixture at ambient temperature. Ethyl acetate was added to the mixtute. The organic layer was separated, and dried over diatomaceous earth. The solvent was removed in vacuo. The residue was purified by silica gel column chromatography eluted with a mixture of n-hexane and ethyl acetate. The fractions were concentrated in vacuo to obtain 2-chloro-5-(1-isopropyl-6-oxo-1,6-dihydro-3-pyridazinyl)-6-phenylnicotinonitrile (160 mg) as white powder.

mp: 191-192° C.; IR (KBr): 2233, 1662 cm⁻¹; ¹H NMR (CDCl₃, δ): 1.35 (6H, d, J=6.6 Hz), 5.33 (1H, 7-plet, J=6.6 Hz), 6.69 (1H, d, J=9.6 Hz), 6.73 (1H, d, J=9.6 Hz), 7.35-7.5 (5H, m), 8.22 (1H, s); APCI/MS: 350[M]⁺; Elemental Analysis for C₁₉H₁₅ClN₄O; Calcd.: C, 65.05; H, 4.31; N, 15.97; Found : C, 65.12; H, 4.31; N, 15.87.

### EXAMPLE 3

A mixture of 2-chloro-5-(1-isopropyl-6-oxo-1,6-dihydro-3-pyridazinyl)-6-phenylnicotinonitrile (110 mg), 28% aq.ammonia (2 ml) and dioxane (2 ml) in sealed tube was stirred at 100° C. for 3 hours. Water (4 ml) was added to the reaction mixture at ambient temperature. EtOAc and water were added to the mixture at ambient temperature. The organic layer was separated, and dried over diatomaceous earth. The solvent was removed in vacuo to give a pale yellow powder. The powder was recrystalized in EtOH to obtain 2-amino-5-(1-isopropyl-6-oxo-1,6-dihydro-3-pyridazinyl)-6-phenylnicotinonitrile (70 mg) as white powder.

mp: 197-198° C.; IR (KBr): 2219, 1641 cm⁻¹; ¹H NMR (CDCl₃, δ): 1.32 (6H, d, J=6.6 Hz), 5.31 (1H, 7-plet, J=6.6 Hz), 5.48 (2H, br), 6.64 (2H, s), 7.3-7.45 (5H, m), 7.97 (1H, s); APCI/MS: 332[M+1]⁺, 354[M+Na]⁺; Elemental Analysis for C₁₉H₁₇N₅O; Calcd.: C, 68.79; H, 5.17; N, 21.13; Found : C, 68.79; H, 5.16; N, 21.38.

### EXAMPLE 4

A mixture of 5-(1-isopropyl-6-oxo-1,6-dihydro-3-pyridazinyl)-2-oxo-6-phenyl-1,2-dihydro-3-pyridine-carbonitrile (190 mg), 30% aq.H₂O₂ (290 μl) and K₂CO₃ (40 mg) in DMSO (1.9 ml) was stirred at 80° C. for 4 days. 1N HCl and EtOAc were added to the reaction mixture at ambient temperature. The organic layer was separated, and dried over diatomaceous earth. The solvent was removed in vacuo. The residue was purified by silica gel column chromatography eluted with a mixture of MeOH and EtOAc. The fractions were concentrated in vacuo to obtain 5-(1-isopropyl-6-oxo-1,6-dihydro-3-pyridazinyl)-2-oxo-6-phenyl-1,2-dihydro-3-pyridinecarboxamide (12 mg) as white powder.

mp: >25⁰° C.; IR (KBr): 3343, 1679, 1650 cm⁻¹; ¹H NMR (CDCl₃, δ): 1.16 (6H, d, J=6.6 Hz), 5.21 (1H, 7-plet, J=6.6 Hz), 5.7-5.8 (1H, br), 6.73 (1H, d, J=9.6 Hz), 6.87 (1H, d, J=9.6 Hz), 7.3-7.6 (5H, m), 8.78 (1H, s), 8.9-9.0 (1H, br), 11.55 (1H, br); API-ES/MS: 373[M+Na]⁺; Elemental Analysis for C₁₉H₁₈N₄O₃.0.3H₂O; Calcd.: C, 64.14; H, 5.27; N, 15.75; Found : C, 64.22; H, 5.21; N, 15.67.

### EXAMPLE 5

A mixture of 2-chloro-5-(1-isopropyl-6-oxo-1,6-dihydro-3-pyridazinyl)-6-phenylnicotinonitrile (2.0 g), 30% aq.H₂O₂ (2.1 ml) K₂CO₃ (315 mg) in DMSO (20 ml) was stirred at ambient temperature for 5 hours. Water and EtOAc were added to the reaction mixture. The organic layer was separated, and dried over diatomaceous earth. The solvent was removed in vacuo. The residue was purified by silica gel column chromatography eluted with a mixture of MeOH and EtOAc. The fractions were concentrated in vacuo to obtain 2-chloro-5-(1-isopropyl-6-oxo-1,6-dihydro-3-pyridazinyl)-6-phenylnicotinamide (1.4 g) as white powder.

mp: 199-200° C.; IR (KBr): 1691, 1658 cm⁻¹; ¹H NMR (CDCl₃, δ): 1.28 (6H, d, J=6.6 Hz), 5.29 (1H, 7-plet, J=6.6 Hz), 6.30 (1H, br), 6.72 (1H, d, J=9.6 Hz), 6.83 (1H, br), 6.84 (1H, d, J=9.6 Hz), 7.3-7.5 (5H, m), 8.47 (1H, s), 8.9-9.0 (1H, br), 11.55 (1H, br); API-ES/MS: 369[M+1]⁺; Elemental Analysis for C₁₉H₁₇ClN₄O₂; Calcd.: C, 61.88; H, 4.65; N, 15.19; Found : C, 62.03; H, 4.66; N, 15.18.

### EXAMPLE 6

A mixture of 2-chloro-5-(1-isopropyl-6-oxo-1,6-dihydro-3-pyridazinyl)-6-phenylnicotinamide (120 mg), 10% Pd/C (24 mg) and ammonium formate (82 mg) in MeOH (2 ml) was stirred at 60° C. for 3 hours. Pd/C was removed by filtration and the solvent was removed in vacuo. Aq.NaHCO₃ solution and EtOAc were added to the residue to give white precipitate. The precipitate was collected by filtration to obtain 5-(1-isopropyl-6-oxo-1,6-dihydro-3-pyridazinyl)-6-phenylnicotinamide (40 mg) as white powder.

mp: 229-230° C.; IR (KBr): 1648, 1629 cm⁻¹; ¹H NMR (CDCl₃, δ): 1.28 (6H, d, J=6.6 Hz), 5.29 (1H, 7-plet, J=6.6 Hz), 6.30 (1H, br), 6.72 (1H, d, J=9.6 Hz), 6.83 (1H, br), 6.84 (1H, d, J=9.6 Hz), 7.3-7.5 (5H, m), 8.47 (1H, s), 8.9-9.0 (1H, br), 11.55 (1H, br); API-ES/MS: 335[M+1]⁺, 357[M+Na]⁺; Elemental Analysis for C₁₉H₁₈N₄O₂.0.2H₂O; Calcd.: C, 67.52; H, 5.49; N, 16.58; Found : C, 67.36; H, 5.37; N, 16.50.

### EXAMPLE 7

2-Amino-5-(1-isopropyl-6-oxo-1,6-dihydro-3-pyridazinyl)-6-phenylnicotinamide was obtained according to a similar manner to that of Example 3.

mp: >250° C.; IR (KBr): 1660, 1627 cm⁻¹; ¹H NMR (CDCl₃, δ): 1.33 (6H, d, J=6.6 Hz), 5.35 (1H, 7-plet, J=6.6 Hz), 5.78 (2H, br), 6.55-6.8 (4H, m), 7.2-7.4 (5H, m), 7.88 (1H, s); API-ES, Negative/MS: 348[M−1]⁺; Elemental Analysis for C₁₉H₁₉N₅O₂.0.1H₂O; Calcd.: C, 64.98; H, 5.51; N, 19.94; Found : C, 65.07; H, 5.58; N, 19.71.

### EXAMPLE 8

A mixture of DMF (2.1 ml) and phosphorus oxychloride (26 μl) was stirred at 0° C. for 30 minutes. 5-(1-Isopropyl-6-oxo-1,6-dihydro-3-pyridazinyl)-6-phenylnicotinamide (100 mg) was added to the reaction mixture. After 1 hour, water and EtOAc were added to the reaction mixture. The organic layer was separated, and dried over diatomaceous earth. The solvent was removed in vacuo. The residue was purified by silica gel column chromatography eluted with a mixture of n-hexane and EtOAc. The fractions were concentrated in vacuo to obtain 5-(1-isopropyl-6-oxo-1,6-dihydro-3-pyridazinyl)-6-phenylnicotinonitrile (60 mg) as white powder.

mp: 133-135° C.; IR (KBr): 2227, 1662 cm⁻¹; ¹H NMR (CDCl₃, δ): 1.36 (6H, d, J=6.6 Hz), 5.34 (1H, 7-plet, J=6.6 Hz), 5.78 (2H, br), 6.68 (1H, d, J=9.6 Hz), 6.75 (1H, d, J=9.6 Hz), 7.3-7.5 (5H, m), 8.22 (1H, d, J=2.0 Hz), 9.00 (1H, d, J=2.0 Hz), API-ES/MS: 317[M+1]⁺, 339[M+Na]⁺; Elemental Analysis for C₁₉H₁₆N₄O; Calcd.: C, 72.14; H, 5.10; N, 17.71; Found : C, 71.96; H, 5.14; N, 17.60.

### EXAMPLE 9

A mixture of 2-chloro-5-(1-isopropyl-6-oxo-1,6-dihydro-3-pyridazinyl)-6-phenylnicotinonitrile (1.0 g), palladium acetate (32 mg), diphenylphosphinopropane (59 mg) and Et₃N (1.19 ml) in DMF (5 ml) and MeOH (10 ml) was stirred at 80° C. under CO gas for 15 hours. Water and EtOAc were added to the reaction mixture. The organic layer was separated, and dried over Na₂SO₄. The solvent was removed in vacuo. The residue was purified by silica gel column chromatography eluted with a mixture of n-hexane and EtOAc. The fractions were concentrated in vacuo to obtain methyl 3-cyano-5-(1-isopropyl-6-oxo-1,6-dihydro-3-pyridazinyl)-6-phenyl-2-pyridinecarboxylate (400 mg) as white powder.

mp: 136-138° C.; IR (KBr): 1741, 1662, 1587 cm⁻¹; ¹H NMR (CDCl₃, δ): 1.36 (6H, d, J=6.6 Hz), 4.10 (3H, s), 5.34 (1H, 7-plet, J=6.6 Hz), 6.70 (1H, d, J=9.6 Hz), 6.78 (1H, d, J=9.6 Hz), 7.3-7.55 (5H, m), 8.40 (1H, s), API-ES/MS: 397[M+Na]⁺; Elemental Analysis for C₂₁H₁₈N₄O₃; Calcd.: C, 67.37; H, 4.85; N, 14.96; Found : C, 67.27; H, 4.83; N, 14.98.

### EXAMPLE 10

A mixture of 2-chloro-5-(1-isopropyl-6-oxo-1,6-dihydro-3-pyridazinyl)-6-phenylnicotinonitrile (100 mg) and NaOMe (46 mg) in DMF was stirred at 100° C. for 15 hours. Water and EtOAc were added to the reaction mixture. The organic layer was separated, and dried over diatomaceous earth. The solvent was removed in vacuo. The residue was purified by silica gel column chromatography eluted with a mixture of n-hexane and EtOAc. The fractions were concentrated in vacuo to obtain 5-(1-isopropyl-6-oxo-1,6-dihydro-3-pyridazinyl)-2-methoxy-6-phenylnicotinonitrile (23 mg) as white powder.

mp: 187-189° C.; IR(KBr): 2227, 1662, 1592cm⁻¹; ¹H NMR (CDCl₃, δ): 1.34 (6H, d, J=6.6 Hz), 4.16 (3H, s), 5.33 (1H, 7-plet, J=6.6 Hz), 6.65 (1H, d, J=9.6 Hz), 6.71 (1H, d, J=9.6 Hz), 7.3-7.5 (5H, m), 8.12 (1H, s), API-ES/MS: 369[M+Na]⁺; Elemental Analysis for C₂₀H₁₈N₄O₂.0.35H₂O; Calcd.: C, 68.11; H, 5.34; N, 15.89; Found : C, 68.35; H, 5.38; N, 15.51.

### EXAMPLE 11

A mixture of methyl 3-cyano-5-(1-isopropyl-6-oxo-1,6-dihydro-3-pyridazinyl)-6-phenyl-2-pyridinecarboxylate (1.0 g), 1N aq.NaOH solution (5 ml) in MeOH (5 ml) was stirred at ambient temperature for 3 hours. 1N HCl was added to the reaction mixture to give pale yellow precipitate. The precipitate was collected by filtration to obtain 3-cyano-5-(1-isopropyl-6-oxo-1,6-dihydro-3-pyridazinyl)-6-phenyl-2-pyridinecarboxylic acid (850 mg) as white powder.

mp: 196-198° C.; IR (KBr): 3453, 1741, 1641, 1569 cm⁻¹; ¹H NMR (DMSO-d₆, δ): 0.98 (6H, d, J=6.6 Hz), 5.02 (1H, 7-plet, J=6.6 Hz), 6.96 (1H, d, J=9.6 Hz), 7.3-7.45 (5H, m), 7.50 (1H, d, J=9.6 Hz), 8.75 (1H, s), 13.0-14.0 (1H, br); API-ES, Negative/MS: 359[M−1]⁺; Elemental Analysis for C₂₀H₁₆N₄O₃.0.1H₂O; Calcd.: C, 66.33; H, 4.51; N, 15.47; Found : C, 68.23; H, 4.56; N, 15.25.

### EXAMPLE 12

A mixture of methyl 3-cyano-5-(1-isopropyl-6-oxo-1,6-dihydro-3-pyridazinyl)-6-phenyl-2-pyridinecarboxylic acid (300 mg), EDCI HCl (207 mg), HOBT (146 mg) in DMF (3 ml) was stirred at ambient temperature for 30 minutes. Ammonium chloride (111 mg) and Et₃N (464 μl) were added to the reaction mixture. After 30 minutes, the reaction mixture was stirred at 70° C. for 2 hours. Water and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 3-cyano-5-(1-isopropyl-6-oxo-1,6-dihydro-3-pyridazinyl)-6-phenyl-2-pyridinecarboxamide (140 mg) as white powder.

mp: 221-223° C.; IR (KBr): 3451, 1700, 1662, 1589 cm⁻¹; ¹H NMR (DMSO-d₆, δ): 1.01 (6H, d, J=6.6 Hz), 5.02 (1H, 7-plet, J=6.6 Hz), 6.96 (1H, d, J=9.7 Hz), 7.3-7.6 (6H, m), 8.06 (1H, br), 8.32 (1H, br), 8.70 (1H, s); API-ES/MS: 360[M+1]⁺, 382[M+Na]⁺; Elemental Analysis for C₂₀H₁₇N₅O₂; Calcd.: C, 66.84; H, 4.77; N, 19.49; Found : C, 66.96; H, 4.79; N, 19.56.

### EXAMPLE 13

A mixture of 5-(1-isopropyl-6-oxo-1,6-dihydro-3-pyridazinyl)-2-oxo-6-phenyl-1,2-dihydro-3-pyridine-carbonitrile (25.0 g) and KOH (16.9 g) in a solution of ethylene glycol (75 ml) and water (37 ml) was stirred at 165° C. After 3 days, the reaction mixture was cooled to ambient temperature. 6N HCl was added to the reaction mixture to appere a white powder. The powder was collected by filtration to obtain 5-(1-isopropyl-6-oxo-1,6-dihydro-3-pyridazinyl)-2-oxo-6-phenyl-1,2-dihydro-3-pyridinecarboxylic acid (26.2 g) as white powder.

mp: 212-215° C.; IR (KBr): 3409, 3318, 1646, 1623, 1581 cm⁻¹; ¹H NMR (DMSO-d₆, δ): 1.02 (6H, d, J=6.7 Hz), 5.00 (1H, 7-plet, J=6.7 Hz), 6.79 (1H, d, J=9.6 Hz), 7.12 (1H, d, J=9.6 Hz), 7.25-7.55 (5H, m), 8.51 (1H, s), 13-15 (2H, br); API-ES, Negative/MS: 350[M−H]⁺.

### EXAMPLE 14

A mixture of 5-(1-isopropyl-6-oxo-1,6-dihydro-3-pyridazinyl)-6-phenylnicotinamide (3.6 g), NaOH (1.18 g) in a mixed solvent of EtOH (20 ml) and water (20 ml) was stirred at 80° C. for 2 hours. Ethanol was removed in vacuo. The residue purified by silica gel column chromatography eluted with a mixture of CHCl₃ and MeOH. The fractions were concentrated in vacuo to obtain 5-(1-isopropyl-6-oxo-1,6-dihydro-3-pyridazinyl)-6-phenyl-nicotinic acid (3.0 g) as white powder.

mp: 221-223° C.; IR (KBr): 3413, 1689, 1652, 1633 cm⁻¹; ¹H NMR (DMSO-d₆, δ): 1.04 (6H, d, J=6.6 Hz), 5.06 (1H, 7-plet, J=6.6 Hz), 6.88 (1H, d,J=9.6 Hz), 7.35 (1H, d, J=9.6 Hz), 7.3-7.5 (5H, m), 8.43 (1 H, d, J=2.0 Hz), 9.20 (1H, d, J=2.0 Hz), 13-14 (1H, br); API-ES, Negative/MS: 354[M−1]⁺.

### EXAMPLE 15

A mixture of methyl 5-(1-isopropyl-6-oxo-1,6-dihydro-3-pyridazinyl)-6-phenylnicotinic acid (150 mg), EDCI HCl (129 mg), HOBT (91 mg), methylamine hydrochloride (45 mg) and Et₃N (94 μl) in DMF (2 ml) was stirred at ambient temperature for 15 hours. Water and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 5-(1-isopropyl-6-oxo-1,6-dihydro-3-pyridazinyl)-N-methyl-6-phenyl-nicotinamide (90 mg) as white powder.

mp: 212-213° C.; IR (KBr): 3369, 1643, 1604, 1579 cm⁻¹; ¹H NMR (DMSO-d₆, δ): 1.01 (6H, d, J=6.6 Hz), 2.85 (3H, d, J=4.5 Hz), 5.04 (1H, 7-plet, J=6.6 Hz), 6.93 (1H, d, J=9.6 Hz), 7.3-7.5 (6H, m), 8.37 (1H, d, J=2.1 Hz), 8.7-8.8 (1H, m), 9.12 (1H, d, J=2.1 Hz); API-ES/MS: 349[M+1]⁺, 371[M+Na]⁺.

### EXAMPLE 16

N-Benzyl-5-(1-isopropyl-6-oxo-1,6-dihydro-3-pyridazinyl)-6-phenylnicotinamide was prepared in a similar manner to that of Example 15.

mp: 205-206° C.; IR (KBr): 3343, 1648, 1600, 1583 cm⁻¹; ¹H NMR (DMSO-d₆, δ): 0.98 (6H, d, J=6.6 Hz), 4.56 (2H, d, J=5.8 Hz), 5.03 (1H, 7-plet, J=6.6 Hz), 6.93 (1H, d, J=9.6 Hz), 7.2-7.5 (11H, m), 8.45 (1H, d, J=2.0 Hz), 9.19 (1H, d, J=2.0 Hz), 9.37 (1H, d, J=5.8 Hz); API-ES/MS: 425[M+1]⁺, 447[M+Na]⁺.

### EXAMPLE 17

5-(1-Isopropyl-6-oxo-1,6-dihydro-3-pyridazinyl)-6-phenyl-N-(2-pyridylmethyl)nicotinamide was prepared in a similar manner to that of Example 15.

mp: 193-194° C.; IR (KBr): 3288, 1662 cm⁻¹; ¹H NMR (DMSO-d₆, δ): 1.00 (6H, d, J=6.6 Hz), 4.65 (2H, d, J=5.7 Hz), 5.03 (1H, 7-plet, J=6.6 Hz), 6.93 (1H, d, J=9.6 Hz), 7.2-7.45 (7H, m), 7.44 (1H, d, J=9.6 Hz), 7.7-7.9 (1H, m), 8.47 (1H, d, J=2.0 Hz), 8.5-8.6 (1H, m), 9.21 (1H, d, J=9.6 Hz), 9.3-9.5 (1H, m); API-ES/MS: 426[M+1]⁺, 448[M+Na]⁺.

### EXAMPLE 18

2-Isopropyl-6-[5-(4-morpholinylcarbonyl)-2-phenyl-3-pyridyl]-3 (2H)-pyridazinone was prepared in a similar manner to that of Example 15.

mp: 132-133° C.; IR (KBr): 3423, 1662, 1621, 1587 cm⁻¹; ¹H NMR (DMSO-d₆, δ): 1.02 (6H, d, J=6.6 Hz), 3.3-3.8 (8H, m), 5.03 (1H, 7-plet, J=6.6 Hz), 6.88 (1H, d, J=9.6 Hz), 7.2-7.45 (6H, m), 8.09 (1H, d, J=2.0 Hz), 8.79 (1H, d, J=2.0 Hz); API-ES/MS: 405[M+1]⁺, 427[M+Na]⁺.

### EXAMPLE 19

2-Isopropyl-6-{5-[(4-methyl-1-piperazinyl)carbonyl]-2-phenyl-3-pyridyl}-3(2H)-pyridazinone was prepared in a similar manner to that of Example 15.

mp: 165-166° C.; IR (KBr): 3421, 1664, 1629, 1587 cm⁻¹; ¹H NMR (DMSO-d₆, δ): 1.02 (6H, d, J=6.6 Hz), 2.21 (3H, m), 2.3-2.5 (4H, m), 3.3-3.8 (4H, m), 5.04 (1H, 7-plet, J=6.6 Hz), 6.88 (1H, d, J=9.6 Hz), 7.2-7.45 (6H, m), 8.06 (1H, d, J=2.0 Hz), 8.76 (1H, d, J=2.0 Hz); API-ES/MS: 418[M+1]⁺, 440[M+Na]⁺.

### EXAMPLE 20

N-(2-Hydroxyethyl)-5-(1-isopropyl-6-oxo-1,6-dihydro-3-pyridazinyl)-6-phenylnicotinamide was prepared in a similar manner to that of Example 15.

mp: 165-167° C.; IR (KBr): 3336, 3295, 1656, 1594 cm⁻¹; ¹H NMR(DMSO-d₆, δ): 0.99 (6H, d, J=6.6 Hz), 3.3-3.65 (4H, m), 4.79 (1H, t, J=5.4 Hz), 5.04 (1H, 7-plet, J=6.6 Hz), 6.93 (1H, d, J=9.6 Hz), 7.3-7.5 (6H, m), 8.40 (1H, d, J=2.0 Hz), 8.82 (1H, d, J=5.4 Hz), 9.14 (1 H, d, J=2.0 Hz); API-ES/MS: 379[M+1]⁺, 401[M+Na]⁺.

### EXAMPLE 21

5-(1-Isopropyl-6-oxo-1,6-dihydro-3-pyridazinyl)-6-phenyl-N-[2-(1-piperadinyl)ethyl]nicotinamide was prepared in a similar manner to that of Example 15.

mp: 92-96° C.; IR (KBr): 3332, 1643, 1583 cm⁻¹; ¹H NMR (DMSO-d₆, δ): 0.9-1.1 (8H, m), 1.2-1.6 (6H, m), 2.3-2.6 (4H, m), 3.3-3.5 (2H, m), 5.04 (1H, 7-plet, J=6.6 Hz), 6.92 (1H, d, J=9.6 Hz), 7.3-7.5 (6H, m), 8.36 (1H, d, J=2.0 Hz), 8.75 (1H, d, J=5.5 Hz), 9.10 (1H, d, J=2.0 Hz); API-ES/MS: 446[M+1]⁺.

### EXAMPLE 22

5-(1-Isopropyl-6-oxo-1,6-dihydro-3-pyridazinyl)-N-[2-(4-morpholinyl)ethyl]-6-phenylnicotinamide was prepared in a similar manner to that of Example 15.

mp: 162-163° C.; IR (KBr): 3367, 1648, 1582 cm⁻¹; ¹H NMR (DMSO-d₆, δ): 1.01 (6H, d, J=6.6 Hz), 2.3-2.6 (6H, m), 3.2-3.7 (6H, m), 5.04 (1H, 7-plet, J=6.6 Hz), 6.92 (1H, d, J=9.6 Hz), 7.2-7.5 (6H, m), 8.36 (1H, d, J=2.0 Hz), 8.77 (1H, d, J=5.5 Hz), 9.11 (1 H, d, J=2.0 Hz); API-ES/MS: 448[M+1]⁺, 470[M+Na]⁺.

### EXAMPLE 23

A mixture of 5-(1-isopropyl-6-oxo-1,6-dihydro-3-pyridazinyl)-6-phenylnicotinic acid (500 mg), 1,3-dicyclohexyl-carbodiimide (307 mg), dimethylaminopyridine (182 mg) and meldrum's acid (215 mg) in CH₂Cl₂ (10 ml) was stirred at ambient temperature for 2 hours. A white precipitate was removed by filtration. The filtrate was evaporated in vacuo to obtain a pale yellow oil. 50% Aq.AcOH solution was added to the residue, which was refluxed with stirring for 12 hours. Aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 6-(5-acetyl-2-phenyl-3-pyridyl)-2-isopropyl-3(2H)-pyridazinone (420 mg) as white powder.

mp: 207-208° C.; ¹H NMR (DMSO-d₆, δ): 1.00 (6H, d, J=6.6 Hz), 2.72 (3H, s), 5.04 (1H, 7-plet, J=6.6 Hz), 6.93 (1H, d, J=9.6 Hz), 7.3-7.5 (6H, m), 8.46 (1H, d, J=2.0 Hz), 9.25 (1H, d, J=2.0 Hz); API-ES/MS: 334[M+l]⁺, 356[M+Na]⁺.

### EXAMPLE 24

A mixture 6-(5-acetyl-2-phenyl-3-pyridyl)-2-isopropyl-3 (2H)-pyridazinone (300 mg) and N,N-dimethylformamide-dimethoxyacetal (1.72 ml) was stirred at 90° C. for 3 hours. The solvent was removed in vacuo to give a yellow powder. EtOH (3 ml) and hydrazine monohydrate (0.4 ml) were added to the residue. The mixture was refluxed with stirring for 12 hours. Water and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 2-isopropyl-6-[2-phenyl-5-(pyrazol-5-yl)-3-pyridyl]-3 (2H)-pyridazinone (90 mg) as pale yellow powder.

mp: 212-213° C.; IR (KBr): 3168, 1664, 1590 cm⁻¹; ¹H NMR (DMSO-d₆, δ): 1.03 (6H, d, J=6.6 Hz), 5.05 (1H, 7-plet, J=6.6 Hz), 6.85-7.0 (2H, m), 7.2-7.5 (6H, m), 7.8-7.9 (1H, m), 8.36 (1H, d, J=2.0 Hz), 9.19 (1H, d, J=2.0 Hz), 13.13 (1H, br); API-ES/MS: 358[M+1]⁺, 380[M+Na]⁺.

### EXAMPLE 25

A mixture of 5-(1-isopropyl-6-oxo-1,6-dihydro-3-pyridazinyl)-6-phenylnicotinic acid (455 mg), diphenylphosphoryl azide (350 μl) and Et₃N (227 μl) in tert-butanol (4 ml) was stirred at 70° C. for 6 hours. Water, aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain tert-butyl 5-(1-isopropyl-6-oxo-1,6-dihydro-3-pyridazinyl)-6-phenyl-3-pyridylcarbamate (120 mg) as white powder.

mp: 221-223° C.; IR (KBr): 3247, 1725, 1660, 1654 cm⁻¹; ¹H NMR (CDCl₃, δ): 1.26 (6H, d, J=6.6 Hz), 1.55 (9H, s), 5.29 (1H, 7-plet, J=6.6 Hz), 6.70 (1H, d, J=9.5 Hz), 6.75 (1H, br), 6.90 (1H, d, J=9.5 Hz), 7.2-7.5 (5H, m), 8.20 (1H, d, J=2.0 Hz), 8.59 (1H, d, J=2 Hz); API-ES/MS: 407[M+1]⁺.

### EXAMPLE 26

A mixture of tert-butyl 5-(1-isopropyl-6-oxo-1,6-dihydro-3-pyridazinyl)-6-phenyl-3-pyridylcarbamate (4.0 g) and 4N HCl in dioxane (50 ml) was stirred at ambient temperature for 4 hours. The solvent was removed in vacuo to give a white powder. EtOAc and aq.NaHCO₃ solution were added to the residue. The organic layer was separated, and dried over diatomaceous earth. The solvent was removed in vacuo to obtain 6-(5-amino-2-phenyl-3-pyridyl)-2-isopropyl-3 (2H)-pyridazinone (2.62 g) as white powder.

mp: 147-148° C.; ¹H NMR (DMSO-d₆, δ): 1.10 (6H, d, J=6.6 Hz), 5.09 (1H, 7-plet, J=6.6 Hz), 6.78 (1H, d, J=9.6 Hz), 7.0-7.4 (7H, m), 8.10 (1H, d, J=2.7 Hz); API-ES/MS: 307[M+1]⁺, 329[M+Na]⁺.

### EXAMPLE 27

A mixture of 6-(5-amino-2-phenyl-3-pyridyl)-2-isopropyl-3 (2H)-pyridazinone (100 mg) and benzoyl chloride (40 μl) in pyridine (2 ml) was stirred at ambient temperature for 2 hours. The solvent was removed in vacuo. The residue was purified by silica gel column chromatography eluted with a mixture of CHCl₃ and MeOH. The fractions were concentrated in vacuo to obtain N-[5-(1-isopropyl-6-oxo-1,6-dihydro-3-pyridazinyl)-6-phenyl-3-pyridyl]benzamide (40 mg) as white powder.

mp: 207-208° C.; IR (KBr): 3307, 1644, 1577 cm⁻¹; ¹H NMR (DMSO-d₆, δ): 1.11 (6H, d, J=6.6 Hz), 5.10 (1H, 7-plet, J=6.6 Hz), 6.86 (1H, d, J=9.6 Hz), 7.22 (1H, d, J=9.6 Hz), 7.3-7.45 (5H, m), 7.45-7.7 (3H, m), 7.9-8.1 (2H, m), 8.45 (1H, d, J=2.4 Hz), 9.13 (1H, d, J=2.4 Hz), 10.7 (1H, br); API-ES/MS: 411[M+1]⁺, 433[M+Na]⁺.

### EXAMPLE 28

N-[5-(1-Isopropyl-6-oxo-1,6-dihydro-3-pyridazinyl)-6-phenyl-3-pyridyl]acetamide was prepared in a similar manner to that of Example 27.

mp: 207-208° C.; IR (KBr): 3421, 1644, 1577 cm⁻¹; ¹H NMR (DMSO-d₆, δ): 1.10 (6H, d, J=6.6 Hz), 2.12 (3H, s), 5.08 (1H, 7-plet, J=6.6 Hz), 6.83 (1H, d, J=9.6 Hz), 7.16 (1H, d, J=9.6 Hz), 7.2-7.45 (5H, m), 8.27 (1H, d, J=2.3 Hz), 8.88 (1H, d, J=2.3 Hz), 10.4 (1H, br);

API-ES/MS: 349[M+1]⁺, 371[M+Na]⁺.

### EXAMPLE 29

A mixture of 6-(5-amino-2-phenyl-3-pyridyl)-2-isopropyl-3 (2H)-pyridazinone (100 mg), 2,5-dimethoxy-tetrahydrofuran (215 μl) and AcOH (0.5 ml) in dioxane (0.5 ml) was stirred at 90° C. for 12 hours. Aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 2-isopropyl-6-[2-phenyl-5-(1H-pyrrol-1-yl)-3-pyridyl]-3(2H)-pyridazinone (30 mg) as white powder.

mp: 189-190° C.; IR (KBr): 3043, 1660, 1587 cm⁻¹; ¹H NMR (DMSO-d₆, δ): 0.96 (6H, d, J=6.6 Hz), 5.01 (1H, 7-plet, J=6.6 Hz), 6.3-6.4 (2H, m), 6.96 (1H, d, J=9.6 Hz), 7.2-7.4 (5H, m), 7.5-7.7 (3H, m), 8.24 (1H, d, J=2.0 Hz), 9.06 (1H, d, J=2.0 Hz); API-ES/MS: 357[M+1]⁺, 379[M+Na]⁺.

### EXAMPLE 30

A mixture of ethyl 2-isopropyl-6-(6-oxo-2-phenyl-1,4,5,6-tetrahydro-3-pyridyl)-3(2H)-pyridazinone (140 g) and manganese(IV) oxide (393 g) in dioxane (1500 ml) was stirred at 75° C. After 24 hours, manganese(IV) oxide (200 g) was added to the reaction mixture. After 3 days, the reaction mixture was cooled to ambient temperature. Manganese oxide was removed by filtration. The filtrate was removed in vacuo. The precipitate was collected by filtration to obtain 2-isopropyl-6-(6-oxo-2-phenyl-1,6-dihydro-3-pyridyl)-3(2H)-pyridazinone (119.4 g) as pale yellow powder.

mp: 107-109° C.; ¹H NMR (DMSO-d₆, δ): 1.02 (6H, d, J=6.6 Hz), 5.00 (1H, 7-plet, J=6.6 Hz), 6.49 (1H, d, J=9.3 Hz), 6.59 (1H, d, J=9.6 Hz), 6.72 (1H, d, J=9.6 Hz), 7.1-7.5 (5H, m), 7.71 (1H, d, J=9.3 Hz), 11.8-12.2 (1H, br); API-ES/MS: 308[M+1]⁺, 330[M+Na]⁺.

### EXAMPLE 31

A mixture of 2-isopropyl-6-(6-oxo-2-phenyl-1,6-dihydro-3-pyridyl)-3 (2H)-pyridazinone (90.2 g), K₂CO₃ (60.9 g) and 2-iodoacetamide (59.8 g) in acetone (900 ml) was stirred at 65° C. After 3 hours, the reaction mixture was cooled at ambient temperature. The excess K₂CO₃ was removed by filtration. The filtrate was removed in vacuo. Water, 1N HCl and EtOAc were added to the residue. The organic layer was separated, and washed with water, aq.NaHCO₃ solution and brine respectively, and dried over Na₂SO₄. The solvent was removed in vacuo. The residue was purified by silica gel column chromatography eluted with a mixture of MeOH and EtOAc (2:100). The fractions were concentrated in vacuo to obtain 2-{[5-(1-isopropyl-6-oxo-1,6-dihydro-3-pyridazinyl)-6-phenyl-2-pyridyl]oxy}acetamide (85.7 g) as pale yellow powder.

mp: 137-138° C.; ¹H NMR (DMSO-d₆, δ): 1.04 (6H, d, J=6.6 Hz), 5.05 (1H, 7-plet, J=6.6 Hz), 6.83 (1H, d, J=9.6 Hz), 7.01 (1H, d, J=8.5 Hz), 7.21 (1H, d, J=9.6 Hz), 7.25-7.55 (7H, m), 7.96 (1H, d, J=8.5 Hz), 11.8-12.2 (1H, br); API-ES/MS: 365[M+1]⁺, 387[M+Na]⁺.

### EXAMPLE 32

A mixture of methyl 2-{[5-(1-isopropyl-6-oxo-1,6-dihydro-3-pyridazinyl)-6-phenyl-2-pyridyl]oxy}acetamide (85 g) and K₂CO₃ (64.5 g) in DMF (850 ml) was stirred at 130° C. After 23 hours, the reaction mixture was cooled to ambient temperature. Water, 1N HCl and EtOAc were added to the residue. The organic layer was separated, and washed with water and brine respectively, and dried over Na₂SO₄. The solvent was removed in vacuo. The precipitate was collected by filtration to obtain 6-(6-amino-2-phenyl-3-pyridyl)-2-isopropyl-3(2H)-pyridazinone (46.6 g) as pale yellow powder.

mp: 167-171° C.; ¹H NMR (DMSO-d₆, δ): 1.04 (6H, d, J=6.6 Hz), 5.03 (1H, 7-plet, J=6.6 Hz), 6.35 (2H, br), 6.54 (1H, d, J=8.5 Hz), 6.72 (1H, d, J=9.6 Hz), 7.05 (1H, d, J=9.5 Hz), 7.25-7.55 (5H, m), 7.61 (1H, d, J=8.5 Hz); API-ES/MS: 307[M+1]⁺, 329[M+Na]⁺.

### EXAMPLE 33

A mixture of 6-(6-amino-2-phenyl-3-pyridyl)-2-isopropyl-3 (2H)-pyridazinone (100 mg) and N-bromosuccinimide (58 mg) in DMF (2 ml) was stirred at 0° C. for 1 hour. Aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 6-(6-amino-5-bromo-2-phenyl-3-pyridyl)-2-isopropyl-3 (2H)-pyridazinone (30 mg) as pale brown powder.

mp: 174-176° C.; IR (KBr): 3419, 3316, 1646, 1621 cm⁻¹; ¹H NMR (DMSO-d₆, δ): 1.02 (6H, d, J=6.6 Hz), 5.01 (1H, 7-plet, J=6.6 Hz), 6.65 (2H, br), 6.75 (1H, d, J=9.6 Hz), 7.15 (1H, d, J=9.6 Hz), 7.2-7.45 (5H, m), 7.94 (1H, s); API-ES/MS: 385[M]⁺, 387[M+2]⁺, 407[M+Na]⁺, 409[M+2+Na]⁺.

### EXAMPLE 34

A mixture of methyl 6-(6-amino-2-phenyl-3-pyridyl)-2-isopropyl-3 (2H)-pyridazinone (13.0 g) and N-chlorosuccinimide (6.8 g) in DMF (100 ml) was stirred at ambient temperature. After 13 hours, the reaction mixture was cooled to ambient temperature. Water and EtOAc were added to the residue. The organic layer was separated, and washed with water, aq.NaHCO₃ solution and brine respectively, and dried over Na₂SO₄. The solvent was removed in vacuo. The residue was purified by silica gel column chromatography eluted with a mixture of MeOH and CHCl₃ (2:100) The fractions were concentrated in vacuo to obtain 6-(6-amino-5-chloro-2-phenyl-3-pyridyl)-2-isopropyl-3 (2H)-pyridazinone (9.0 g) as pale yellow crystal.

mp: 207-208° C.; IR (KBr): 3409, 3318, 1646, 1623, 1581 cm⁻¹; ¹H NMR (DMSO-d₆, δ): 1.02 (6H, d, J=6.6 Hz), 5.02 (1H, 7-plet, J=6.6 Hz), 6.72 (2H, br), 6.76 (1H, d, J=9.6 Hz), 7.14 (1H, d, J=9.6 Hz), 7.25-7.55 (5H, m), 7.81 (1H, s); API-ES/MS: 341[M+H]⁺, 343[M+2+H]⁺; Elemental Analysis for C₁₈H₁₇ClN₄O; Calcd.: C, 63.44; H, 5.03; N, 16.44; Found : C, 63.53; H, 4.99; N, 16.62.

### EXAMPLE 35

A mixture of 6-(6-amino-2-phenyl-3-pyridyl)-2-isopropyl-3 (2H)-pyridazinone (1.25 g) and N-iodosuccinimide (918 mg) in DMF (12.5 ml) was stirred at ambient temperature for 1 hour. Aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 6-(6-amino-5-iodo-2-phenyl-3-pyridyl)-2-isopropyl-3 (2H)-pyridazinone (977 mg) as pale brown powder.

mp: 196-197° C.; IR (KBr): 3274, 1648, 1621, 1581 cm⁻¹; ¹H NMR (DMSO-d₆, δ): 1.02 (6H, d, J=6.6 Hz), 5.01 (1H, 7-plet, J=6.6 Hz), 6.48 (2H, br), 6.75 (1H, d, J=9.6 Hz), 7.15 (1H, d, J=9.6 Hz), 7.2-7.45 (5H, m), 8.08 (1H, s); API-ES/MS: 433[M+1]⁺, 455[M+Na]⁺.

### EXAMPLE 36

A mixture of 6-(6-amino-5-iodo-2-phenyl-3-pyridyl)-2-isopropyl-3 (2H)-pyridazinone (100 mg), phenylboric acid (34 mg), 2 M aq.Na₂CO₃ solution (0.693 ml) and tetrakistriphenylphosphine palladium (27 mg) in DME (1.0 ml) was stirred at 80° C. for 13 hours. Aq.NaHCO₃ solution and EtOAc were added to the reaction mixture. The organic layer was separated, and dried over diatomaceous earth. The solvent was removed in vacuo. The residue was purified by silica gel column chromatography eluted with a mixture of CHCl₃ and MeOH. The fractions were concentrated in vacuo to obtain 6-(6-amino-2,5-diphenyl-3-pyridyl)-2-isopropyl-3 (2H)-pyridazinone (71 mg) as white powder.

mp: 208-209° C.; IR (KBr): 3286, 1658, 1621, 1587 cm⁻¹; ¹H NMR (DMSO-d₆, δ): 1.01 (6H, d, J=6.6 Hz), 5.01 (1H, 7-plet, J=6.6 Hz), 6.75 (1H, d, J=9.6 Hz), 7.23 (1H, d, J=9.6 Hz), 7.25-7.6 (11H, m); API-ES/MS: 383[M+1]⁺, 405[M+Na]⁺.

### EXAMPLE 37

6-[6-Amino-S-(4-fluorophenyl)-2-phenyl-3-pyridyl]-2-isopropyl-3 (2H)-pyridazinone was prepared in a similar manner to that of Example 36.

mp: 216-218° C.; IR (KBr): 3123, 1666, 1627, 1589 cm⁻¹; ¹H NMR (DMSO-d₆, δ): 1.00 (6H, d, J=6.6 Hz), 5.00 (1H, 7-plet, J=6.6 Hz), 6.07 (2H, br), 6.76 (1H, d, J=9.6 Hz), 7.23 (1H, d, J=9.6 Hz), 7.25-7.7 (10H, m); API-ES/MS: 401[M+1]⁺, 423[M+Na]⁺.

### EXAMPLE 38

6-(2-Amino-6-phenyl-3,3′-bipyrid-yl)-2-isopropyl-3 (2H)-pyridazinone was prepared in a similar manner to that of Example 36.

mp: 236-238° C.; IR (KBr): 3330, 1650, 1623, 1581 cm⁻¹; ¹H NMR (DMSO-d₆, δ): 1.00 (6H, d, J=6.6 Hz), 5.00 (1H, 7-plet, J=6.6 Hz), 6.21 (2H, br), 6.78 (1H, d, J=9.6 Hz), 7.2-7.6 (6H, m), 7.60 (1H, s), 7.9-8.05 (1H, m), 8.5-8.8 (2H, m); API-ES, Negative/MS: 382[M−1]⁺.

### EXAMPLE 39

A mixture of 2-amino-5-(1-isopropyl-6-oxo-1,6-dihydro-3-pyridazinyl)-6-phenylnicotinonitrile (1.0 g) andthioacetamide (227 mg)in DMF (5 ml) and 4N HCl in dioxane (5 ml) was stirred at 110° C. After 6 hours, thioacetamide (677 mg) was added to the reaction mixture, which was stirred at 110° C. for 2 hours. 1N NaOH was added to the reaction mixture to afford a precipitate. The precipitate was collected by filtration. The precipitate was purified by silica gel column chromatography eluted with a mixture of CHCl₃ and MeOH. The fractions were concentrated in vacuo to obtain 2-amino-5-(1-isopropyl-6-oxo-1,6-dihydro-3-pyridazinyl)-6-phenyl-3-pyridinecarbothioamide (890 mg) as pale yellow powder.

¹H NMR (DMSO-d₆, δ): 0.95 (6H, d, J=6.6 Hz), 4.99 (1H, 7-plet, J=6.6 Hz), 6.84 (1H, d, J=9.5 Hz), 7.2-7.5 (8H, m), 7.78 (1H, s), 9.65 (1H, br), 9.93 (1H, br); API-ES/MS: 366[M+H]⁺, 388[M+Na]⁺.

### EXAMPLE 40

A mixture of 2-amino-5-(1-isopropyl-6-oxo-1,6-dihydro-3-pyridazinyl)-6-phenyl-3-pyridinecarbothioamide (100 mg) and phenacylbromide (55 mg) in dioxane (2 ml) was stirred at 90° C. After 2 hours, water and aq.NaHCO₃ solution were added to the reaction mixture. The aqueous mixture was extracted with EtOAc. The organic layer was dried over earth granular. The solvent was removed in vacuo to give a precipitate. The precipitate was purified by silica gel column chromatography eluted with a mixture of CHCl₃ and MeOH. The fractions were concentrated in vacuo to obtain 6-[6-amino-2-phenyl-5-(4-phenyl-thiazol-2-yl)-3-pyridyl]-2-isopropyl-3(2H)-pyridazinone (53 mg) as pale yellow powder.

IR (KBr): 3384, 1670, 1629, 1587 cm⁻¹; ¹H NMR (DMSO-d₆, δ): 1.04 (6H, d, J=6.6 Hz), 5.04 (1H, 7-plet, J=6.6 Hz), 6.83 (1H, d, J=9.6 Hz), 7.2-7.6 (9H, m), 7.9-8.1 (2H, m), 8.19 (1H, s), 8.23 (1H, s); API-ES/MS: 466[M+H]⁺, 488[M+Na]⁺.

### EXAMPLE 41

6-[6-Amino-5-(4-methyl-thiazol-2-yl)-2-phenyl-3-pyridyl]-2-isopropyl-3(2H)-pyridazinone was obtained according to a similar manner to that of Example 40.

IR (KBr): 3355, 1664, 1621, 1587 cm⁻¹; ¹H NMR (DMSO-d₆, δ): 1.04 (6H, d, J=6.6 Hz), 2.47 (3H, s), 5.03 (1H, 7-plet, J=6.6 Hz), 6.80 (1H, d, J=9.6 Hz), 7.26 (1H, d, J=9.6 Hz), 7.3-7.5 (6H, m), 8.00 (2H, br), 8.10 (1H, s); API-ES/MS: 404[M+H]⁺, 426[M+Na]⁺.

### EXAMPLE 42

A mixture of 5-(1-isopropyl-6-oxo-1,6-dihydro-3-pyridazinyl)-6-phenylnicotinic acid (1.0 g)and H₂SO₄ (0.3 ml) in MeOH (20 ml) was refluxed with stirring for 4 days. The pH of the reaction mixture was adjusted to 7.0 with aq. NaHCO₃ solution. The aqueous mixture was extracted with EtOAc. The organic layer was dried over earth granular. The solvent was removed in vacuo to give a paste. The paste was triturated with IPE to afford a powder, which was collected by filtration to give methyl 5-(1-isopropyl-6-oxo-1,6-dihydro-3-pyridazinyl)-6-phenyl-nicotinate (901 mg) as pale yellow powder.

IR (KBr): 1724, 1670, 1594 cm⁻¹; ¹H NMR (CDCl₃, δ): 1.32 (6H, d, J=6.7 Hz), 1.85-2.15 (4H, m), 3.5-3.8 (4H, m), 5.32 (1H, 7-plet, J=6.7 Hz), 6.67 (1H, d, J=9.6 Hz), 6.80 (1H, d, J=9.5 Hz), 7.25-7.5 (5H, m), 8.13 (1H, d, J=2.2 Hz), 8.91 (1H, d, J=2.2 Hz); API-ES/MS: 389[M+H]⁺, 411[M+Na]⁺.

### EXAMPLE 43

2-Isopropyl-6-[2-phenyl-5-(1-pyrrolidinylcarbonyl)-3-pyridyl]-3 (2H)-pyridazinone was obtained according to a similar manner to that of Example 15.

IR (KBr): 1660, 1608, 1585 cm⁻¹; ¹H NMR (CDCl₃, δ): 1.32 (6H, d, J=6.7 Hz), 1.85-2.15 (4H, m), 3.5-3.8 (4H, m), 5.32 (1H, 7-plet, J=6.7 Hz), 6.67 (1H, d, J=9.6 Hz), 6.80 (1H, d, J=9.5 Hz), 7.25-7.5 (5H, m), 8.13 (1H, d, J=2.2 Hz), 8.91 (1H, d, J=2.2 Hz); API-ES/MS: 389[M+H]⁺, 411[M+Na]⁺.

### EXAMPLE 44

N-Butyl-5-(1-isopropyl-6-oxo-1,6-dihydro-3-pyridazinyl)-6-phenylnicotinamide was obtained according to a similar manner to that of Example 15.

IR (KBr): 3365, 1646, 1600, 1583 cm⁻¹; ¹H NMR (CDCl₃, δ): 1.00 (3H, t, J=7.2 Hz), 1.29 (6H, d, J=6.6 Hz), 1.35-1.55 (2H, m), 1.55-1.8 (2H, m), 3.53 (2H, q, J=6.0 Hz), 5.30 (1H, 7-plet, J=6.6 Hz), 6.31 (1H, br), 6.69 (1H, d, J=9.5 Hz), 6.84 (1H, d, J=9.5 Hz), 7.3-7.5 (5H, m), 8.34 (1H, d, J=2.1 Hz), 9.04 (1H, d, J=2.1 Hz); API-ES/MS: 391[M+H]⁺, 413[M+Na]⁺.

### EXAMPLE 45

A solution of 5-(1-isopropyl-6-oxo-1,6-dihydro-3-pyridazinyl)-2-oxo-6-phenyl-1,2-dihydro-3-pyridinecarboxylic acid (2.5 g) in quinoline (10 ml) was stirred at 230° C. After 2 days, the reaction mixture was cooled to 25° C. Water and CHCl₃ were added to the reaction mixture. The organic layer was separated, washed with water, dried over earth granular. The solvent was removed in vacuo. The residue was purified by silica gel column chromatography eluted with a mixture of CHCl₃ and MeOH. The fractions were concentrated in vacuo to obtain 2-isopropyl-6-(6-oxo-2-phenyl-1,6-dihydro-3-pyridyl)-3 (2H)-pyridazinone (1.27 g) as pale yellow powder.

¹H NMR (DMSO-d₆, δ): 1.02 (6H, d, J=6.6 Hz), 5.00 (1H, 7-plet, J=6.6 Hz), 6.49 (1H, d, J=9.4 Hz), 6.73 (1H, d, J=9.6 Hz), 7.00 (1H, d, J=9.6 Hz), 7.2-7.5 (5H, m), 6.71 (1H, d, J=9.4 Hz), 11.9 (1H, br); API-ES/MS: 330[M+Na]⁺.

### EXAMPLE 46

A mixture of methyl 2-isopropyl-6-(6-oxo-2-phenyl-1,6-dihydro-3-pyridyl)-3(2H)-pyridazinone (1.0 g) and Et₃N HCl (537 mg) in phosphorus oxychloride (1.8 ml) was stirred at 100° C. for 2 hours. The solvent was removed in vacuo to give an oily residue. Water was added slowly to the residue, which was extracted with EtOAc. The organic layer was dried over earth granular. The solvent was removed in vacuo to give 6-(6-chloro-2-phenyl-3-pyridyl)-2-isopropyl-3 (2H)-pyridazinone (880 mg) as pale yellow powder.

IR (KBr): 1666, 1590 cm⁻¹; ¹H NMR(DMSO-d₆, δ): 1.02 (6H, d, J=6.6 Hz), 5.04 (1H, 7-plet, J=6.6 Hz), 6.87 (1H, d, J=9.5 Hz), 7.2-7.5 (6H, m), 7.66 (1H, d, J=8.3 Hz), 8.11 (1H, d, J=8.3 Hz); API-ES/MS: 326[M+H]⁺, 348[M+Na]⁺.

### EXAMPLE 47

Methyl 5-(1-isopropyl-6-oxo-1,6-dihydro-3-pyridazinyl)-2-oxo-6-phenyl-1,2-dihydro-3-pyridinecarboxylate was obtained according to a similar manner to that of Example 42.

IR (KBr): 3411, 1741, 1662 cm⁻¹; ¹H NMR (DMSO-d₆, δ): 1.02 (6H, d, J=6.6 Hz), 3.79 (3H, s), 5.00 (1H, 7-plet, J=6.6 Hz), 6.75 (1H, d, J=9.6 Hz), 7.08 (1H, d, J=9.6 Hz), 7.2-7.5 (5H, m), 8.24 (1H, s), 12.48 (1H, br); API-ES/MS: 367[M+H]⁺, 389[M+Na]⁺.

### EXAMPLE 48

Methyl 2-chloro-5-(1-isopropyl-6-oxo-1,6-dihydro-3-pyridazinyl)-6-phenylnicotinate was obtained according to a similar manner to that of Example 46.

IR (KBr): 1739, 1662, 1590 cm⁻¹; ¹H NMR (DMSO-d₆, δ): 1.01 (6H, d, J=6.6 Hz), 3.93 (3H, s), 5.04 (1H, 7-plet, J=6.6 Hz), 6.90 (1H, d, J=9.6 Hz), 7.2-7.5 (6H, m), 8.47 (1H, s); API-ES/MS: 384[M+H]⁺, 406[M+Na]⁺.

### EXAMPLE 49

A solution of methyl 5-(1-isopropyl-6-oxo-1,6-dihydro-3-pyridazinyl)-6-phenylnicotinate (700 mg) in THF (10 ml) was stirred at 5° C. LiBH₄ (44 mg) was added to the solution and the reaction mixture was stirred at 25° C. for 18 hours. Water and CHCl₃ were added to the reaction mixture. The organic layer was separated, washed with water, dried over earth granular. The solvent was removed in vacuo. The residue was purified by silica gel column chromatography eluted with a mixture of CHCl₃ and MeOH. The fractions were concentrated in vacuo to obtain 6-[5-(hydroxymethyl)-2-phenyl-3-pyridyl]-2-isopropyl-3 (2H)-pyridazinone.

IR (KBr): 3372, 1644, 1577 cm⁻¹; ¹H NMR (DMSO-d₆, δ): 1.31 (6H, d, J=6.6 Hz), 2.2-2.35 (1H, m), 4.86 (2H, d, J=5.6 Hz), 5.31 (1H, 7-plet, J=6.6 Hz), 6.67 (1H, d, J=9.6 Hz), 6.80 (1H, d, J=9.6 Hz), 7.2-7.5 (5H, m), 7.94 (1H, d, J=2 Hz), 8.73 (1H, d, J=2 Hz); API-ES/MS: 324[M+H]⁺, 346[M+Na]⁺.

### EXAMPLE 50

Methyl 2-carbamoylmethoxy-5-(1-isopropyl-6-oxo-1,6-dihydro-3-pyridazinyl)-6-phenylnicotinate was obtained according to a similar manner to that of Example 31.

mp: 183-184° C.; IR (KBr): 3407, 1716, 1691, 1668, 1589 cm⁻¹; ¹H NMR (DMSO-d₆, δ): 1.02 (6H, d, J=6.6 Hz), 3.88 (3H, s), 4.87 (2H, s), 5.04 (1H, 7-plet, J=6.6 Hz), 6.87 (1H, d, J=9.6 Hz), 7.2-7.5 (8H, m), 8.36 (1H, s); API-ES/MS: 423[M+H]⁺, 445[M+Na]⁺.

### EXAMPLE 51

A mixture of methyl 2-chloro-5-(1-isopropyl-6-oxo-1,6-dihydro-3-pyridazinyl)-6-phenylnicotinate (1.0 g) and 1N aq.NaOH solution (5 ml) in DME (10 ml) was stirred at 25° C. After 3 hours, the solvent was removed in vacuo to give a residue. 1N HCl solution (5 ml) was added to the reaction mixture to afford a precipitate. The precipitate was collected by filtration to obtain 2-chloro-5-(1-isopropyl-6-oxo-1,6-dihydro-3-pyridazinyl)-6-phenyl-nicotinic acid (800 mg) as pale yellow powder.

¹H NMR (DMSO-d₆, δ): 1.01 (6H, d, J=6.6 Hz), 5.04 (1H, 7-plet, J=6.6 Hz), 6.90 (1H, d, J=9.6 Hz), 7.2-7.5 (6H, m), 8.43 (1H, s), 13.9 (1H, br); API-ES/MS: 368[M−1]⁺, 370[M+1]⁺.

### EXAMPLE 52

A mixture of 6-(5-acetyl-2-phenyl-3-pyridyl)-2-isopropyl-3 (2H)-pyridazinone (1.0 g) and N,N-dimethylformamide-dimethoxyacetal (4 ml) was stirred at 95° C. for 6 hours. The solvent was removed in vacuo to give a yellow powder. EtOH (8 ml) and methylhydrazine (0.8 ml) were added to the residue. The mixture was refluxed with stirring for 3 hours. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was recrystalized with EtOH to give 2-isopropyl-6-[5-(1-methyl-1H-pyrazol-5-yl)-2-phenyl-3-pyridyl]-3(2H)-pyridazinone (400 mg) as pale yellow crystal.

¹H NMR (DMSO-d₆, δ): 1.00 (6H, d, J=6.6 Hz), 3.99 (3H, s), 5.03 (1H, 7-plet, J=6.6 Hz), 6.68 (1H, d, J=1.9 Hz), 6.92 (1H, d, J=9.6 Hz), 7.2-7.5 (5H, m), 7.46 (1H, d, J=9.6 Hz), 7.56 (1H, d, J=1.9 Hz), 8.21 (1H, d, J=2 Hz), 8.94 (1H, d, J=2 Hz); API-ES/MS: 372[M+1]⁺, 394[M+Na]⁺.

### EXAMPLE 53

A mixture of 6-(5-amino-2-phenyl-3-pyridyl)-2-isopropyl-3 (2H)-pyridazinone (290 mg) and sodium nitrate (80 mg) in 50% aq.H₂SO₄ solution (1.5 ml) was stirred at 25° C. for 1 hour. The reaction mixture was added to the ACOH (5 ml) at 100° C., which was stirred under same conditions. After 20 minutes, the reaction mixture was cooled to 25° C. The reaction mixture was poured into aq.NaHCO₃ solution. The aqueous solution was extracted with EtOAc. The organic layer was washed with brine, dried over Na₂SO₄. The solvent was removed in vacuo to give a pale yellow powder. The powder was collected by filtration to give 6-(5-hydroxy-2-phenyl-3-pyridyl)-2-isopropyl-3(2H)-pyridazinone (100 mg) as pale yellow crystal.

IR (KBr): 3444, 1671, 1589 cm⁻¹; ¹H NMR (DMSO-d₆, δ): 1.07 (6H, d, J=6.6 Hz), 3.88 (3H, s), 5.06 (1H, 7-plet, J=6.6 Hz), 6.81 (1H, d, J=9.6 Hz), 7.01 (1H, d, J=9.6 Hz), 7.2-7.5 (6H, m), 8.31 (1H, d, J=2.6 Hz), 10.31 (1H, br); API-ES/MS: 308[M+H]⁺, 330[M+Na]⁺.

### EXAMPLE 54

A mixture of 6-(5-hydroxy-2-phenyl-3-pyridyl)-2-isopropyl-3 (2H)-pyridazinone (300 mg), N-(3-bromopropyl)phthalimide (287 mg) and NaH (43 mg) in DMF (5 ml) was stirred at 25° C. After 24 hours, the reaction mixture was poured into water. The aqueous solution was extracted with EtOAc. The organic layer was washed with brine, dried over Na₂SO₄. The solvent was removed in vacuo to give a pale yellow powder. The powder was collected by filtration to give 2-(3-{[5-(1-isopropyl-6-oxo-1,6-dihydro-3-pyridazinyl)-6-phenyl-3-pyridyl]oxy}propyl)-1H-isoindole-1,3 (2H)-dione (300 mg) as pale yellow powder.

¹H NMR (DMSO-d₆, δ): 0.9-1.1 (6H, m), 2.0-2.2 (2H, s), 3.7-3.9 (2H, m), 4.1-4.3 (2H, m), 5.03 (1H, 7-plet, J=6.6 Hz), 6.86 (1H, d, J=9.6 Hz), 7.2-7.5 (7H, m), 7.7-7.9 (4H, m), 8.2-8.41 (1H, m); API-ES/MS: 495[M+H]⁺, 517[M+Na]⁺.

### EXAMPLE 55

A mixture of 2-(3-{[5-(1-isopropyl-6-oxo-1,6-dihydro-3-pyridazinyl)-6-phenyl-3-pyridyl]oxy}propyl)-1H-isoindole-1, 3 (2H)-dione (200 mg) and hydrazine monohydrate (0.6 ml) in EtOH (15 ml) was stirred at 80° C. After 12 hours, the reaction mixture was poured into aq.NaHCO₃ solution. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was dissolved in EtOAc. 4N HCl in EtOAc (0.135 ml) was added to the solution to give a precipitate. The precipitate was collected by filtration to give 6-[5-(3-aminopropoxy)-2-phenyl-3-pyridyl]-2-isopropyl-3(2H)-pyridazinone dihydrochloride (50 mg) as pale yellow powder.

¹H NMR (DMSO-d₆, δ): 0.99 (6H, d, J=6.6 Hz), 2.0-2.25 (2H, m), 2.8-3.1 (2H, br), 4.2-4.4 (2H, m), 5.03 (1H, 7-plet, J=6.6 Hz), 5.8-6.3 (2H, br), 6.91 (1H, d, J=9.6 Hz), 7.2-7.5 (6H, m), 7.76 (1H, d, J=2.7 Hz), 8.1-8.5 (2H, br), 8.52 (1H, d, J=2.7 Hz), API-ES/MS: 365[M-2HCl+H]⁺.

### EXAMPLE 56

A mixture of 2-isopropyl-6-(6-oxo-2-phenyl-1,6-dihydro-3-pyridyl)-3 (2H)-pyridazinone (300 mg), K₂CO₃ (405 mg) and 2-bromo-N,N-diethylethylamine hydrobromide (280 mg) in DMF (3 ml) was stirred at 60° C. After 12 hours, the reaction mixture was poured into water. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was dissolved in ethyl acetate. 4N HCl in EtOAc (0.394 ml) was added to the solution to give a precipitate. The precipitate was collected by filtration to give 6-{6-[2-(diethylamino)ethoxy]-2-phenyl-3-pyridyl}-2-isopropyl-3(2H)-pyridazinone dihydrochloride (100 mg) as pale yellow powder.

¹H NMR (DMSO-d₆, δ): 0.9-1.4 (12H, m), 2.7-3.7 (6H, m), 3.8-4.8 (2H, m), 4.85-5.3 (1H, m), 6.6-8.1 (9H, m), 10.6-11.0 (2H, m); API-ES/MS: 407[M−2HCl+H]⁺.

### EXAMPLE 57

A mixture of 6-(6-chloro-2-phenyl-3-pyridyl)-2-isopropyl-3 (2H)-pyridazinone (1.0 g), Pd/C (200 mg) and ammoniumformate (968 mg) in MeOH (20 ml) was stirred at 45° C. After 5 hours, the Pd/C was removed by filtration. The filtrate was evaporated in vacuo to give an oily residue. Aq.NaHCO₃ solution was added to the residue. The aqueous solution was extracted with EtOAc. The organic layer was washed with brine, dried over Na₂SO₄. The solvent was removed in vacuo to give a pale yellow residue. The precipitate was collected by filtration to give 2-isopropyl-6-(2-phenyl-3-pyridyl)-3 (2H)-pyridazinone (612 mg) as pale yellow oil.

¹H NMR (DMSO-d₆, δ): 1.03 (6H, d, J=6.6 Hz), 5.05 (1H, 7-plet, J=6.6 Hz), 6.86 (1H, d, J=9.6 Hz), 7.2-7.45 (6H, m), 7.53 (1H, dd, J=7.7 Hz and 4.8 Hz), 8.04 (1H, dd, J=7.7 Hz and 1.5 Hz), 8.74 (1H, dd, J=4.8 Hz and 1.5 Hz); API-ES/MS: 292[M+H]⁺, 314[M+Na]⁺.

### EXAMPLE 58

A mixture of 2-isopropyl-6-(6-oxo-2-phenyl-1,6-dihydro-3-pyridyl)-3 (2H)-pyridazinone (500 mg), N-(2-bromoethyl)-phthalimide (465 mg) and K₂CO₃ (450 mg) in DMF (5 ml) was stirred at 25° C. After 24 hours, the reaction mixture was poured into water. The aqueous solution was extracted with EtOAc. The organic layer was washed with brine, dried over Na₂SO₄. The solvent was removed in vacuo to give a pale yellow powder. The powder was collected by filtration to give 2-(2-{[5-(1-isopropyl-6-oxo-1,6-dihydro-3-pyridazinyl)-6-phenyl-2-pyridyl]oxy}ethyl)-1H-isoindole-1,3 (2H)-dione (500 mg) as pale yellow powder.

¹H NMR (DMSO-d₆, δ): 1.04 (6H, d, J=6.6 Hz), 4.02 (2H, t, J=5.3 Hz), 4.66 (2H, t, J=5.3 Hz), 5.03 (1H, 7-plet, J=6.6 Hz), 6.78 (1H, d, J=9.6 Hz), 6.84 (1H, d, J=8.5 Hz), 7.09 (1H, d, J=9.6 Hz), 7.2-7.5 (5H, m), 7.7-8.0 (2H, m), 7.87 (1H, d, J=8.5 Hz); API-ES/MS: 481[M+H]⁺, 503[M+Na]⁺.

### EXAMPLE 59

A mixture of 2-(2-{[5-(1-isopropyl-6-oxo-1,6-dihydro-3-pyridazinyl)-6-phenyl-2-pyridyl]oxy}ethyl)-1H-isoindole-1,3 (2H)-dione (400 mg) and hydrazine monohydrate (0.6 ml) in EtOH (15 ml) was stirred at 80° C. After 12 hours, the reaction mixture was poured into aq.NaHCO₃ solution. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of chloroform and methanol. The fractions were concentrated in vacuo to afford a yellow powder. The powder was collected by filtration to give 6-[6-(2-aminoethoxy)-2-phenyl-3-pyridyl]-2-isopropyl-3 (2H)-pyridazinone (200 mg) as pale yellow powder.

¹H NMR (DMSO-d₆, δ): 1.05 (6H, d, J=6.6 Hz), 2.8-3.0 (2H, m), 3.1-3.3 (2H, br), 4.2-4.4 (2H, m), 5.05 (1H, 7-plet, J=6.6 Hz), 6.81 (1H, d, J=9.6 Hz), 6.93 (1H, d, J=8.5 Hz), 7.18 (1H, d, J=9.6 Hz), 7.25-7.5 (5H, m), 7.92 (1H, d, J=8.5 Hz); API-ES/MS: 351[M+H]⁺, 373[M+Na]⁺.

### EXAMPLE 60

A mixture of 2-{[5-(1-isopropyl-6-oxo-1,6-dihydro-3-pyridazinyl)-6-phenyl-2-pyridyl]oxy}acetamide (190 mg) and N,N-dimethylformamide-dimethoxyacetal (1 ml) was stirred at 90° C. After 2 hours, the reaction mixture was evaporated in vacuo to give a powder. Hydrazine monohydrate (0.2 ml) and AcOH (2 ml) were added to the residue, which was stirred at 25° C. for 24 hours. Aq.NaHCO₃ solution was added to the reaction mixture. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was collected by filtration to give 2-isopropyl-6-[2-phenyl-6-(1H-1,2,4-triazol-5-ylmethoxy)-3-pyridyl]-3 (2H)-pyridazinone (30 mg) as white powder.

¹H NMR (DMSO-d₆, δ): 1.05 (6H, d, J=6.6 Hz), 5.05 (1H, 7-plet, J-6.6 Hz), 5.51 (2H, s), 6.82 (1H, d, J=9.6 Hz), 7.00 (1H, d, J=8.5 Hz), 7.21 (1H, d, J=9.6 Hz), 7.25-7.45 (5H, m), 7.96 (1H, d, J=8.5 Hz), 8.38 (1H, br); API-ES/MS: 389[M+H]⁺, 411[M+Na]⁺.

### EXAMPLE 61

2-(3-{[5-(1-Isopropyl-6-oxo-1,6-dihydro-3-pyridazinyl)-6-phenyl-2-pyridyl]oxy}propyl)-1H-isoindole-1,3 (2H)-dione was prepared in a similar manner to that of Example 58.

¹H NMR (DMSO-d₆, δ): 1.05 (6H, d, J=6.6 Hz), 2.0-2.2 (2H, m), 3.78 (2H, t, J=6.6 Hz), 4.41 (2H, t, J=5.9 Hz), 5.05 (1H, 7-plet, J=6.6 Hz), 6.74 (1H, d, J=8.5 Hz), 6.81 (1H, d, J=9.6 Hz), 7.17 (1H, d, J=9.6 Hz), 7.2-7.4 (5H, m), 7.7-8.0 (5H, m), 8.2-8.41 (1H, m); API-ES/MS: 495[M+H]⁺, 517[M+Na]⁺.

### EXAMPLE 62

6-[6-(3-Aminopropoxy)-2-phenyl-3-pyridyl]-2-isopropyl-3 (2H)-pyridazinone dihydrochloride was prepared in a similar manner to that of Example 55.

¹H NMR (DMSO-d₆, δ): 1.05 (6H, d, J=6.6 Hz), 1.7-2.0 (2H, m), 2.6-2.8 (2H, m), 3.0-3.5 (2H, br), 4.3-4.5 (2H, m), 5.05 (1H, 7-plet, J=6.6 Hz), 6.81 (1H, d, J=9.6 Hz), 6.92 (1H, d, J=8.4 Hz), 7.18 (1H, d, J=9.6 Hz), 7.2-7.5 (5H, m), 7.91 (1H, d, J=8.4 Hz); API-ES/MS: 365[M+H]⁺.

### EXAMPLE 63

A mixture of 6-(6-amino-2-phenyl-3-pyridyl)-2-isopropyl-3 (2H)-pyridazinone (150 mg) and nicotinoyl chloride hydrochloride (87 mg) in pyridine (3 ml) was stirred at 25° C. After 12 hours, the reaction mixture was evaporated in vacuo to give an oily residue. Water was poured into the residue. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was collected by filtration to give N-[5-(1-isopropyl-6-oxo-1,6-dihydro-3-pyridazinyl)-6-phenyl-2-pyridyl]-nicotinamide (100 mg) as white powder.

IR (KBr): 3421, 1683, 1664, 1590 cm⁻¹; ¹H NMR (DMSO-d₆, δ): 1.05 (6H, d, J=6.6 Hz), 5.06 (1H, 7-plet, J=6.6 Hz), 6.8-6.9 (1H, m), 7.2-7.8 (7H, m), 8.0-8.5 (3H, m), 8.7-8.85 (1H, m), 9.1-9.3 (1H, m), 11.3 (1H, br); API-ES/MS: 412[M+H]⁺, 434[M+Na]⁺.

### EXAMPLE 64

N-[5-(1-Isopropyl-6-oxo-1,6-dihydro-3-pyridazinyl)-6-phenyl-2-pyridyl]benzamide was prepared in a similar manner to that of Example 63.

IR (KBr): 3421, 1689, 1654, 1590 cm⁻¹; ¹H NMR (DMSO-d₆, δ): 1.03 (6H, d, J=6.6 Hz), 5.05 (1H, 7-plet, J=6.6 Hz), 6.85 (1H, d, J=9.6 Hz), 7.29 (1H, d, J=9.6 Hz), 7.3-7.7 (9H, m), 8.0-8.4 (2H, m), 10.9 (1H, br); API-ES/MS: 411[M+H]⁺, 433[M+Na]⁺.

### EXAMPLE 65

N-[5-(1-Isopropyl-6-oxo-1,6-dihydro-3-pyridazinyl)-6-phenyl-2-pyridyl]acetamide was prepared in a similar manner to that of Example 63.

IR (KBr): 3239, 1693, 1650, 1589 cm⁻¹; ¹H NMR (DMSO-d₆, δ): 1.02 (6H, d, J=6.6 Hz), 2.14 (3H, s), 5.03 (1H, 7-plet, J=6.6 Hz), 6.83 (1H, d, J=9.6 Hz), 7.25 (1H, d, J=9.6 Hz), 7.3-7.6 (5H, m), 8.02 (1H, d, J=8.5 Hz), 8.17 (1H, d, J=8.5 Hz), 10.7 (1H, br); API-ES/MS: 349[M+H]⁺, 371[M+Na]⁺.

### EXAMPLE 66

N-[5-(1-Isopropyl-6-oxo-1,6-dihydro-3-pyridazinyl)-6-phenyl-2-pyridyl]-2,2-dimethylpropanamide was prepared in a similar manner to that of Example 63.

IR (KBr): 3259, 1693, 1658, 1590 cm⁻¹; ¹H NMR (DMSO-d₆, δ): 1.02 (6H, d, J=6.6 Hz), 1.27 (9H, s), 5.04 (1H, 7-plet, J=6.6 Hz), 6.83 (1H, d, J=9.6 Hz), 7.25 (1H, d, J=9.6 Hz), 7.3-7.6 (5H, m), 8.02 (1H, d, J=8.6 Hz), 8.17 (1H, d, J=8.6 Hz), 9.97 (1H, br); API-ES/MS: 391[M+H]⁺, 413[M+Na]⁺.

### EXAMPLE 67

A mixture of 6-(6-amino-2-phenyl-3-pyridyl)-2-isopropyl-3 (2H)-pyridazinone (150 mg), 4-pyridinecarbaldehyde (51 μl), ACOH (31 μl) and NaBH(OAc)₃ (145 mg) in CH₂Cl₂ (5 ml) was stirred at 25° C. After 12 hours, the reaction mixture was evaporated in vacuo to give an oily residue. Aq.NaHCO₃ solution was poured into the residue. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was collected by filtration to give 2-isopropyl-6-{2-phenyl-6-[(4-pyridylmethyl)amino]-3-pyridyl}-3(2H)-pyridazinone (100 mg) as white powder.

¹H NMR (DMSO-d₆, δ): 1.03 (6H, d, J=6.6 Hz), 4.58 (2H, d, J=6.0 Hz), 5.03 (1H, 7-plet, J=6.6 Hz), 6.84 (1H, d, J=8.5 Hz), 6.73 (1H, d, J=9.6 Hz), 7.06 (1H, d, J=9.6 Hz), 7.1-7.4 (6H, m), 7.6-7.7 (1H, m), 8.5-8.6 (1H, m), 9.97 (1H, br); API-ES/MS: 398[M+H]⁺, 420[M+Na]⁺.

### EXAMPLE 68

2-Isopropyl-6-{2-phenyl-6-[(3-pyridylmethyl)amino]-3-pyridyl}-3 (2H)-pyridazinone was prepared in a similar manner to that of Example 67.

¹H NMR (DMSO-d₆, δ): 1.03 (6H, d, J=6.6 Hz), 4.57 (2H, d, J=5.9 Hz), 5.03 (1H, 7-plet, J=6.6 Hz), 6.63 (1H, d, J=8.6 Hz), 6.73 (1H, d, J=9.6 Hz), 7.06 (1H, d, J=9.6 Hz), 7.1-7.4 (6H, m), 7.5-7.8 (3H, m), 8.4-8.6 (2H, m); API-ES/MS: 398[M+H]⁺, 420[M+Na]⁺.

### EXAMPLE 69

2-Isopropyl-6-{2-phenyl-6-[(2-pyridylmethyl)amino]-3-pyridyl}-3 (2H)-pyridazinone was prepared in a similar manner to that of Example 67.

¹H NMR (DMSO-d₆, δ): 1.03 (6H, d, J=6.6 Hz), 4.65 (2H, d, J=5.9 Hz), 5.03 (1H, 7-plet, J=6.6 Hz), 6.67 (1H, d, J=8.6 Hz), 6.73 (1H, d, J=9.6 Hz), 7.06 (1H, d, J=9.6 Hz), 7.1-7.4 (7H, m), 7.5-7.8 (3H, m), 8.4-8.6 (1H, m); API-ES/MS: 398[M+H]⁺, 420[M+Na]⁺.

### EXAMPLE 70

6-[6-(Benzylamino)-2-phenyl-3-pyridyl]-2-isopropyl-3 (2H)-pyridazinone was prepared in a similar manner to that of Example 67.

IR (KBr): 3413, 1656, 1592 cm⁻¹; ¹H NMR (DMSO-d₆, δ): 1.03 (6H, d, J=6.6 Hz), 4.55 (2H, d, J=5.9 Hz), 5.03 (1H, 7-plet, J=6.6 Hz), 6.60 (1H, d, J=8.6 Hz), 6.73 (1H, d, J=9.6 Hz), 7.06 (1H, d, J=9.6 Hz), 7.1-7.9 (12H, m); API-ES/MS: 397[M+H]⁺, 419[M+Na]⁺.

### EXAMPLE 71

6-[2-(4-Fluorophenyl)-6-oxo-1,6-dihydro-3-pyridyl]-2-isopropyl-3(2H)-pyridazinone was prepared in a similar manner to that of Example 30.

¹H NMR (DMSO-d₆, δ): 0.8-1.3 (6H, m), 5.04 (1H, 7-plet, J=6.6 Hz), 6.15 (2H, br), 6.94 (1H, d, J=9.5 Hz), 7.1-7.4 (5H, m), 7.47 (1H, d, J=9.5 Hz); API-ES/MS: 326[M+H]⁺, 348[M+Na]⁺.

### EXAMPLE 72

2-{[6-(4-Fluorophenyl)-5-(1-isopropyl-6-oxo-1,6-dihydro-3-pyridazinyl)-2-pyridyl]oxy}acetamide was prepared in a similar manner to that of Example 31.

¹H NMR (DMSO-d₆, δ): 1.07 (6H, d, J=6.6 Hz), 4.76 (2H, s), 5.06 (1H, 7-plet, J=6.6 Hz), 6.87 (1H, d, J=9.5 Hz), 7.01 (1H, d, J=8.5 Hz), 7.1-7.4 (6H, m), 7.4-7.6 (1H, br), 7.96 (1H, d, J=8.5 Hz); API-ES/MS: 405[M+Na]⁺.

### EXAMPLE 73

6-[6-Amino-2-(4-fluorophenyl)-3-pyridyl]-2-isopropyl-3 (2H)-pyridazinone was prepared in a similar manner to that of Example 32.

¹H NMR (DMSO-d₆, δ): 1.04 (6H, d, J=6.6 Hz), 5.04 (1H, 7-plet, J=6.6 Hz), 6.37 (2H, br), 6.54 (1H, d, J=8.5 Hz), 6.76 (1H, d, J=9.6 Hz), 7.0-7.4 (5H, m), 7.60 (1H, d, J=8.5 Hz); API-ES/MS: 325[M+H]⁺, 347[M+Na]⁺.

### EXAMPLE 74

6-[6-Amino-5-chloro-2-(4-fluorophenyl)-3-pyridyl]-2-isopropyl-3 (2H)-pyridazinone was prepared in a similar manner to that of Example 34.

¹H NMR(DMSO-d₆, δ): 1.02 (6H, d, J=6.6 Hz), 5.02 (1H, 7-plet, J=6.6 Hz), 6.74 (2H, br), 6.80 (1H, d, J=9.5 Hz), 7.0-7.4 (5H, m), 7.81 (1H, s); API-ES/MS: 359[M+H]⁺, 381[M+Na]⁺.

### EXAMPLE 75

6-(5-Amino-6-chloro-2-phenyl-3-pyridyl)-2-isopropyl-3 (2H)-pyridazinone was prepared in a similar manner to that of Example 34.

¹H NMR (DMSO-d₆, δ): 1.07 (6H, d, J=6.6 Hz), 4.76 (2H, s), 5.06 (1H, 7-plet, J=6.6 Hz), 6.87 (1H, d, J=9.5 Hz), 7.01 (1H, d, J=8.5 Hz), 7.1-7.4 (6H, m), 7.4-7.6 (1H, br), 7.96 (1H, d, J=8.5 Hz); API-ES/MS: 341[M+H]⁺, 393[M+Na]⁺.

### EXAMPLE 76

A mixture of 6-(6-chloro-2-phenyl-3-pyridyl)-2-isopropyl-3 (2H)-pyridazinone (1.0 g) and hydrazine monohydrate (3 ml) in dioxane (151 ml) was heated in a sealed tube at 150° C. After 4 days, the solvent was removed in vacuo to give a precipitate. The precipitate was purified by silica gel column chromatography eluted with a mixture of CHCl₃ and MeOH. The fractions were concentrated in vacuo to obtain 6-(6-hydrazino-2-phenyl-3-pyridyl)-2-isopropyl-3 (2H)-pyridazinone (300 mg) as pale yellow powder.

¹H NMR (DMSO-d₆, δ): 1.04 (6H, d, J=6.6 Hz), 4.27 (2H, br), 5.03 (1H, 7-plet, J=6.6 Hz), 6.74 (1H, d, J=9.6 Hz), 6.80 (1H, d, J=8.6 Hz), 7.2-7.4 (5H, m), 7.68 (1H, d, J=8.6 Hz), 7.8-7.9 (1H, m); API-ES/MS: 322[M+H]⁺, 344[M+Na]⁺.

### EXAMPLE 77

A mixture of 6-(5-amino-2-phenyl-3-pyridyl)-2-isopropyl-3 (2H)-pyridazinone (120 mg) and N-chlorosuccinimide (111 mg) in DMF (2.4 ml) was stirred at 25° C. After 13 hours, water and EtOAc were added to the residue. The organic layer was separated, and washed with water, aq.NaHCO₃ solution and brine respectively, and dried over Na₂SO₄. The solvent was removed in vacuo. The residue was purified by silica gel column chromatography eluted with a mixture of MeOH and CHCl₃ (2:100). The fractions were concentrated in vacuo to give pale yellow powder. The precipitate was recrystalized with ethanol to obtain 6-(5-amino-4,6-dichloro-2-phenyl-3-pyridyl)-2-isopropyl-3(2H)-pyridazinone (100 mg) as pale yellow powder.

mp: 186-188° C.; IR (KBr): 3322, 1652, 1623, 1585 cm⁻¹; ¹H NMR (DMSO-d₆, δ): 0.8-1.3 (6H, d, m), 5.04 (1H, 7-plet, J=6.6 Hz), 6.15 (2H, br), 6.94 (1H, d, J=9.5 Hz), 7.1-7.4 (5H, m), 7.47 (1H, d, J=9.5 Hz); API-ES/MS: 375[M]⁺, 377[M+2]⁺, 397[M+Na]⁺, 399[M+2+Na]⁺.

### EXAMPLE 78

A mixture of 6-[(E)-1-benzoyl-2-(dimethylamino)vinyl]-2-methyl-3 (2H)-pyridazinone (185 g), cyanoacetamide (60.4 g) and 28% NaOMe in MeOH solution (282 ml) in DMF (370 ml) was refluxed with stirring. After 4 hours, water was added to the reaction mixture at 25° C. Concentrated HCl solution (130 ml) was added to the reaction mixture to afford a precipitate. The precipitate was collected by filtration to obtain 5-(1-methyl-6-oxo-1,6-dihydro-3-pyridazinyl)-2-oxo-6-phenyl-1,2-dihydro-3-pyridine-carbonitrile (150 g) as pale yellow powder.

¹H NMR (DMSO-d₆, δ): 3.59 (3H, s), 6.97 (1H, d, J=9.6 Hz), 6.73 (1H, d, J=9.6 Hz), 7.3-7.6 (5H, m), 8.37 (1H, s); API-ES/MS: 327[M+Na]⁺.

### EXAMPLE 79

5-(1-Methyl-6-oxo-1,6-dihydro-3-pyridazinyl)-2-oxo-6-phenyl-1,2-dihydro-3-pyridinecarboxylic acid was prepared in a similar manner to that of Example 13.

¹H NMR (DMSO-d₆, δ): 3.65 (3H, s), 6.6-6.7 (2H, m), 7.3-7.6 (5H, m), 8.51 (1H, s), 13-15 (2H, br); API-ES, Negative/MS: 322[M−H]⁺.

### EXAMPLE 80

2-Methyl-6-(6-oxo-2-phenyl-1,6-dihydro-3-pyridyl)-3 (2H)-pyridazinone was prepared in a similar manner to that of Example 45.

¹H NMR (DMSO-d₆, δ): 3.67 (3H, s), 6.5-6.7 (3H, m), 7.2-7.9 (6H, m), 11.9 (1H, br); API-ES/MS: 280[M+H]⁺, 302[M+Na]⁺.

### EXAMPLE 81

A mixture of 2-methyl-6-(6-oxo-2-phenyl-1,6-dihydro-3-pyridyl)-3 (2H)-pyridazinone (10 g), 2-iodoacetamide (6.62 g) and K₂CO₃ (19.8 g) in DMF (80 ml) was stirred at 25° C. After 4 hours, the reaction mixture was stirred at 130° C. After 72 hours, water was added to the reaction mixture. The aqueous solution was extracted with EtOAc. The organic phase was separated, dried over Na₂SO₄. The solvent was removed in vacuo to give a brown precipitate. The precipitate was collected by filtration to obtain 6-(6-amino-2-phenyl-3-pyridyl)-2-methyl-3 (2H)-pyridazinone (5.9 g) as pale yellow powder.

¹H NMR (DMSO-d₆, δ): 3.67 (3H, s), 6.39 (2H, br), 6.54 (1H, d, J=8.5 Hz), 6.62 (1H, d, J=9.6 Hz), 6.72 (1H, d, J=9.6 Hz), 7.2-7.4 (5H, m), 7.60 (1H, d, J=8.5 Hz); API-ES/MS: 279[M+H]⁺, 301[M+Na]⁺.

### EXAMPLE 82

6-(6-Amino-5-chloro-2-phenyl-3-pyridyl)-2-methyl-3 (2H)-pyridazinone was prepared in a similar manner to that of Example 34.

IR (KBr): 3413, 1648, 1577 cm⁻¹; ¹H NMR (DMSO-d₆, δ): 3.64 (3H, s), 6.66 (1H, d, J=9.6 Hz), 6.7-6.9 (3H, m), 7.2-7.5 (5H, m), 7.78 (1H, s); API-ES/MS: 313[M+H]⁺, 335[M+Na]⁺.

### EXAMPLE 83

6-[2-(2-Bromophenyl)-6-oxo-1,6-dihydro-3-pyridyl]-2-isopropyl-3 (2H)-pyridazinone was prepared in a similar manner to that of Example 30.

IR (KBr): 3438, 1650, 1592 cm⁻¹; ¹H NMR (CDCl₃, δ): 0.8-1.0 (6H, m), 4.93 (1H, d, J=6.6 Hz), 6.4-6.6 (1H, m), 6.81 (1H, d, J=9.6 Hz), 7.2-7.5 (2H, m), 7.6-7.9 (2H, m); API-ES/MS: 408[M+Na]⁺, 410[M+2+Na]⁺.

### EXAMPLE 84

6-[6-Amino-2-(2-bromophenyl)-3-pyridyl]-2-isopropyl-3 (2H)-pyridazinone was prepared in a similar manner to that of Example 81.

IR (KBr): 3403, 1654, 1587 cm⁻¹; ¹H NMR (CDCl₃, δ): 0.7-1.0 (6H, m), 4.93 (1H, d, J=6.6 Hz), 6.40 (2H, br), 6.57 (1H, d, J=9.6 Hz), 6.79 (1H, d, J=9.6 Hz), 7.1-8.0 (6H, m); API-ES/MS: 385[M]⁺, 387[M+2]⁺, 407[M+Na]⁺, 409[M+2+Na]⁺.

### EXAMPLE 85

6-[6-Amino-2-(2-bromophenyl)-5-chloro-3-pyridyl]-2-isopropyl-3 (2H)-pyridazinone was prepared in a similar manner to that of Example 34.

IR (KBr): 3471, 1662, 1627, 1587 cm⁻¹; ¹H NMR(CDCl₃, δ): 0.7-1.0 (6H, br), 4.92 (1H, d, J=6.6 Hz), 6.6-6.9 (3H, m), 7.1-8.0 (6H, m); API-ES/MS: 441[M+Na]⁺, 443[M+2+Na]⁺.

### EXAMPLE 86

A mixture of 3-chloro-5-(6-methoxy-3-pyridazinyl)-6-phenyl-2-pyridinamine (100 g) and 6N HCl (0.4 ml) in 4N HCl in dioxane solution (2 ml) was stirred at 70° C. After 2 hours, aq.NaHCO₃ solution was added to the reaction mixture at 25° C. The aqueous solution was extracted with EtOAc. The organic layer was separated, dried over earth granular. The solvent was removed in vacuo to give a precipitate. The precipitate was collected by filtration to obtain 6-(6-amino-5-chloro-2-phenyl-3-pyridyl)-3 (2H)-pyridazinone (35 g) as pale yellow powder.

IR (KBr): 3324, 1677, 1662, 1579 cm⁻¹; ¹H NMR (CDCl₃, δ): 4.01 (3H, s), 6.62 (1H, d, J=10 Hz), 6.73 (2H, br), 6.83 (1H, d, J=10 Hz), 7.2-7.5 (5H, m), 7.74 (1H, s), 13.0 (1H, br); API-ES/MS: 321[M+Na]⁺, 323[M+2+Na]⁺.

### EXAMPLE 87

A mixture of 6-(6-amino-5-chloro-2-phenyl-3-pyridyl)-3 (2H)-pyridazinone (200 mg) and NaH (28 mg)in DMF (2 ml) was stirred at 25° C. After 1 hour, ethyliodide (110 mg) was added to the reaction mixture, which was stirred at 25° C. for 2 hours. Water was added to the reaction mixture, which was extracted with EtOAc. The EtOAc phase was separated, dried over earth granular. The solvent was removed in vacuo to give oily residue. The residue was purified by silica gel column chromatography eluted with a mixture of CHCl₃ and MeOH. The fractions were concentrated in vacuo to obtain 6-(6-amino-5-chloro-2-phenyl-3-pyridyl)-2-ethyl-3 (2H)-pyridazinone (90 mg) as white powder.

¹H NMR (CDCl₃, δ): 1.12 (3H, t, J=7.2 Hz), 4.00 (2H, q, J=7.2 Hz), 6.71 (1H, d, J=9.6 Hz), 6.75 (2H, br), 6.94 (1H, d, J=9.6 Hz), 7.2-7.5 (5H, m), 7.80 (1H, s); API-ES/MS: 355[M+H]⁺, 357[M+2+H]⁺, 377[M+Na]⁺.

### EXAMPLE 88

6-(6-Amino-5-chloro-2-phenyl-3-pyridyl)-2-pentyl-3 (2H)-pyridazinone was prepared in a similar manner to that of Example 87.

¹H NMR (CDCl₃, δ): 0.87 (3H, t, J=6.6 Hz), 1.1-1.6 (6H, m), 3.96 (2H, q, J=7.1 Hz), 6.71 (1H, d, J=9.6 Hz), 6.75 (2H, br), 6.95 (1H, d, J=9.6 Hz), 7.2-7.5 (5H, m), 7.77 (1H, s); API-ES/MS: 369[M+H]⁺, 391[M+Na]⁺, 393[M+2+Na]⁺.

### EXAMPLE 89

6-(6-Amino-5-chloro-2-phenyl-3-pyridyl)-2-butyl-3 (2H)-pyridazinone was prepared in a similar manner to that of Example 87.

¹H NMR (CDCl₃, δ): 0.88 (3H, t, J=6.6 Hz), 1.0-1.6 (2H, m), 3.97 (2H, q, J=7.1 Hz), 6.71 (1H, d, J=9.6 Hz), 6.75 (2H, br), 6.96 (1H, d, J=9.6 Hz), 7.2-7.5 (5H, m), 7.77 (1H, s); API-ES/MS: 355[M+H]⁺, 357[M+2+H]⁺, 377[M+Na]⁺, 379[M+2+Na]⁺.

### EXAMPLE 90

6-(6-Amino-5-chloro-2-phenyl-3-pyridyl)-2-propyl-3 (2H)-pyridazinone was prepared in a similar manner to that of Example 87.

¹H NMR (CDCl₃, δ): 0.80 (3H, t, J=7.5 Hz), 1.4-1.7 (2H, m), 3.94 (2H, q, J=7.1 Hz), 6.71 (1H, d, J=9.6 Hz), 6.75 (2H, br), 6.94 (1H, d, J=9.6 Hz), 7.2-7.5 (5H, m), 7.78 (1H, s); API-ES/MS: 343[M+2+H]⁺, 363[M+Na]⁺, 365[M+2+Na]⁺.

### EXAMPLE 91

6-(6-Amino-5-chloro-2-phenyl-3-pyridyl)-2-benzyl-3 (2H)-pyridazinone was prepared in a similar manner to that of Example 87.

¹H NMR (CDCl₃, δ): 5.18 (2H, s), 6.7-6.85 (3H, m), 6.96 (1H, d, J=9.6 Hz), 7.1-7.5 (10H, m), 7.74 (1H, s); API-ES/MS: 389[M+H]⁺, 411[M+Na]⁺, 413[M+2+Na]⁺.

### EXAMPLE 92

6-(6-Amino-5-chloro-2-phenyl-3-pyridyl)-2-isobutyl-3 (2H)-pyridazinone was prepared in a similar manner to that of Example 87.

¹H NMR (CDCl₃, δ): 0.80 (6H, d, J=6.7 Hz), 1.8-2.1 (1H, m), 3.82 (2H, d, J=7.3 Hz), 6.7-6.9 (3H, m), 6.94 (1H, d, J=9.6 Hz), 7.2-7.4 (5H, m), 7.74 (1H, s); API-ES/MS: 355[M+H]⁺, 357[M+2+H]⁺, 3779[M+Na]⁺, 379[M+2+Na]⁺.

### EXAMPLE 93

2-[3-(6-Amino-5-chloro-2-phenyl-3-pyridyl)-6-oxo-1 (6H)-pyridazinyl]acetamide was prepared in a similar manner to that of Example 87.

¹H NMR (CDCl₃, δ): 4.63 (2H, s), 6.6-6.9 (2H, m), 7.1-7.6 (7H, m), 7.71 (1H, s); API-ES/MS: 356[M+H]⁺, 378[M+Na]⁺, 380[M+2+Na]⁺.

### EXAMPLE 94

6-(6-Amino-5-chloro-2-phenyl-3-pyridyl)-2-[2-(4-methyl-1-piperazinyl)-2-oxoethyl]-3 (2H)-pyridazinone was prepared in a similar manner to that of Example 15.

¹H NMR (CDCl₃, δ): 2.21 (3H, s), 2.2-2.5 (2H, m), 3.3-3.6 (2H, m), 4.99 (2H, s), 6.6-6.9 (4H, m), 7.2-7.5 (5H, m), 7.68 (1H, s); API-ES/MS: 439[M+H]⁺, 441[M+2+H]⁺, 461[M+Na]⁺.

### EXAMPLE 95

Methyl [3-(6-amino-5-chloro-2-phenyl-3-pyridyl)-6-oxo-1 (6H)-pyridazinyl]acetate was prepared in a similar manner to that of Example 87.

¹H NMR (CDCl₃, δ): 3.70 (3H, s), 4.87 (2H, s), 6.7-6.9 (4H, m), 7.2-7.5 (5H, m), 7.74 (1H, s); API-ES/MS: 371[M+H]⁺, 393[M+Na]⁺, 395[M+2+Na]⁺.

### EXAMPLE 96

A mixture of methyl [3-(6-amino-5-chloro-2-phenyl-3-pyridyl)-6-oxo-1 (6H)-pyridazinyl]acetate (3.26 mg) and 1N aq.NaOH solution (15 ml) in MeOH (15 ml) was stirred at 25° C. After 3 hours, water was added to the reaction mixture to give a precipitate. The precipitate was collected by filtration to obtain [3-(6-amino-5-chloro-2-phenyl-3-pyridyl)-6-oxo-1 (6H)-pyridazinyl]acetic acid (2.7 g) as white powder.

¹H NMR (CDCl₃, δ): 4.76 (2H, s), 6.7-6.9 (4H, m), 7.2-7.5 (5H, m), 7.72 (1H, s), 13.1 (1H, br); API-ES, Negative/MS: 355[M−H]⁺, 357[M−H+2]⁺.

### EXAMPLE 97

6-(6-Amino-5-chloro-2-phenyl-3-pyridyl)-2-(2-oxo-2-phenylethyl)-3(2H)-pyridazinone was prepared in a similar manner to that of Example 87.

¹H NMR (CDCl₃, δ): 5.67 (2H, s), 6.7-6.9 (4H, m), 7.2-7.8 (10H, m), 8.0-8.15 (1H, m); API-ES/MS: 417[M+H]⁺, 419[M+2+H]⁺, 439[M+Na]⁺, 441[M+2+Na]⁺.

### EXAMPLE 98

2-[3-(6-Amino-5-chloro-2-phenyl-3-pyridyl)-6-oxo-1 (6H)-pyridazinyl]-N-(2-hydroxyethyl)acetamide was prepared in a similar manner to that of Example 15.

¹H NMR (CDCl₃, δ): 3.1-3.6 (4H, m), 4.6-4.8 (3H, s), 6.6-6.9 (4H, m), 7.2-7.5 (5H, m), 7.70 (1H, s), 8.0-8.2 (1H, m); API-ES/MS: 400[M+H]⁺, 422[M+Na]⁺, 424[M+2+Na]⁺.

### EXAMPLE 99

6-(6-Amino-5-chloro-2-phenyl-3-pyridyl)-2-[2-oxo-2-(1-pyrrolidinyl)ethyl]-3(2H)-pyridazinone was prepared in a similar manner to that of Example 15.

¹H NMR (CDCl₃, δ): 1.6-2.01 (4H, m), 3.2-3.6 (4H, m), 4.89 (2H, s), 6.6-6.9 (4H, m), 7.2-7.5 (5H, m), 7.95 (1H, s); API-ES/MS: 410[M+H]⁺, 4121[M+2+H]⁺, 432[M+Na]⁺, 434[M+2+Na]⁺.

### EXAMPLE 100

6-(6-Amino-5-chloro-2-phenyl-3-pyridyl)-2-[2-oxo-2-(1-piperidinyl)ethyl]-3(2H)-pyridazinone was prepared in a similar manner to that of Example 15.

¹H NMR (CDCl₃, δ): 1.3-1.7 (6H, m), 3.3-3.5 (4H, m), 4.96 (2H, s), 6.6-6.9 (4H, m), 7.2-7.5 (5H, m), 7.68 (1H, s); API-ES/MS: 424[M+H]⁺, 443[M+Na]⁺, 448[M+2+Na]⁺.

### EXAMPLE 101

2-[3-(6-Amino-5-chloro-2-phenyl-3-pyridyl)-6-oxo-1(6H)-pyridazinyl]-N-propylacetamide was prepared in a similar manner to that of Example 15.

¹H NMR(CDCl₃, δ): 0.86 (3H, t, J=7.3 Hz), 1.3-1.6 (2H, m), 2.9-3.2 (2H, m), 4.66 (2H, s), 6.6-6.9 (2H, m), 7.2-7.5 (5H, m), 7.70 (1H, s), 8.0-8.2 (1H, m); API-ES/MS: 398[M+H]⁺, 420[M+Na]⁺, 422[M+2+Na]⁺.

### EXAMPLE 102

6-(6-Amino-5-chloro-2-phenyl-3-pyridyl)-2-[2-(4-morpholinyl)-2-oxoethyl]-3(2H)-pyridazinone was prepared in a similar manner to that of Example 15.

¹H NMR (CDCl₃, δ): 3.4-3.7 (8H, m), 5.00 (2H, s), 6.6-6.9 (4H, m), 7.2-7.5 (5H, m), 7.68 (1H, s); API-ES/MS: 426[M+H]⁺, 448[M+Na]⁺, 450[M+2+Na]⁺.

### EXAMPLE 103

1-Isopropyl-2′-phenyl-3,3′-bipyridine-6,6′(1H,1′H)-dione was prepared in a similar manner to that of Example 30.

¹H NMR (DMSO-d₆, δ): 0.97 (6H, d, J=6.6 Hz), 4.87 (1H, 7-plet, J=6.6 Hz), 6.2-6.3 (1H, m), 6.35-6.5 (1H, m), 7.05-7.6 (8H, m), 11.8 (1H, br); API-ES/MS: 307[M+H]⁺, 329[M+Na]⁺.

### EXAMPLE 104

2-Isopropyl-6-[2-(4-methoxyphenyl)-6-oxo-1,6-dihydro-3-pyridyl]-3(2H)-pyridazinone was prepared in a similar manner to that of Example 30.

¹H NMR (DMSO-d₆, δ): 1.11 (6H, d, J=6.6 Hz), 3.76 (3H, s), 5.05 (1H, 7-plet, J=6.6 Hz), 6.44 (1H, d, J=9.3 Hz), 6.71 (1H, d, J=9.6 Hz), 6.8-7.0 (3H, m), 7.15-7.3 (2H, m), 7.67 (1H, d, J=9.3 Hz), 11.8 (1H, br); API-ES/MS: 338[M+H]⁺, 360[M+Na]⁺.

### EXAMPLE 105

6-[5-Chloro-2-(4-methoxyphenyl)-6-oxo-1,6-dihydro-3-pyridyl]-2-isopropyl-3(2H)-pyridazinone was prepared in a similar manner to that of Example 34.

¹H NMR (DMSO-d₆, δ): 1.10 (6H, d, J=6.6 Hz), 3.77 (3H, s), 5.03 (1H, 7-plet, J=6.6 Hz), 6.73 (1H, d, J=9.6 Hz), 6.85-7.0 (3H, m), 7.15-7.3 (2H, m), 7.95 (1H, s), 12.5 (1H, br); API-ES/MS: 394[M+Na]⁺.

### EXAMPLE 106

6-[6-Amino-5-chloro-2-(4-methoxyphenyl)-3-pyridyl]-2-isopropyl-3(2H)-pyridazinone was prepared in a similar manner to that of Example 81.

¹H NMR (DMSO-d₆, δ): 1.10 (6H, d, J=6.6 Hz), 3.75 (3H, s), 5.04 (1H, 7-plet, J=6.6 Hz), 6.6-7.3 (8H, m), 7.05-7.35 (2H, m), 7.75 (1H, s ); API-ES/MS: 371[M+H]⁺, 393[M+Na]⁺, 395[M+2+Na]⁺.

### EXAMPLE 107

A mixture of 6-[6-amino-5-chloro-2-(4-methoxyphenyl)-3-pyridyl]-2-isopropyl-3(2H)-pyridazinone (100 mg) and 1N borontribromide in CH₂Cl₂ solution (0.54 ml) in CH₂Cl₂ (5 ml) was stirred at 25° C. After 12 hours, the reaction mixture was evaporated in vacuo to give an oily residue. Aq.NaHCO₃ solution was poured into the residue. The aqueous solution was extracted with EtOAc. The organic layer was washed with brine, dried over Na₂SO₄. The solvent was removed in vacuo to give a pale yellow residue. The residue was purified by silica gel column chromatography eluted with a mixture of CHCl₃ and MeOH. The fractions were concentrated in vacuo to afford a yellow powder. The powder was collected by filtration to give 6-[6-amino-5-chloro-2-(4-hydroxyphenyl)-3-pyridyl]-2-isopropyl-3 (2H)-pyridazinone (60 mg) as white powder.

¹H NMR (DMSO-d₆, δ): 1.15 (6H, d, J=6.6 Hz), 5.07 (1H, 7-plet, J=6.6 Hz), 6.6-6.75 (5H, m), 6.9-7.2 (3H, m), 7.72 (5H, s), 9.60 (1H, br); API-ES, Negative/MS: 355[M−H]⁺, 357[M+2−H]⁺.

### EXAMPLE 108

2-Isopropyl-6-[2-(2-methoxyphenyl)-6-oxo-1,6-dihydro-3-pyridyl]-3(2H)-pyridazinone was prepared in a similar manner to that of Example 30

¹H NMR (DMSO-d₆, δ): 0.99 (6H, d, J=6.6 Hz), 3.58 (3H, s), 4.97 (1H, 7-plet, J=6.6 Hz), 6.44 (1H, d, J=9.4 Hz), 6.72 (1H, d, J=9.6 Hz), 6.9-7.5 (5H, m), 7.68 (1H, d, J=9.4 Hz), 11.8 (1H, br); API-ES/MS: 338[M+H]⁺, 360[M+Na]⁺.

### EXAMPLE 109

6-[5-Chloro-2-(2-methoxyphenyl)-6-oxo-1,6-dihydro-3-pyridyl]-2-isopropyl-3(2H)-pyridazinone was prepared in a similar manner to that of Example 34.

¹H NMR (DMSO-d₆, δ): 0.97 (6H, d, J=6.6 Hz), 3.59 (3H, s), 4.95 (1H, 7-plet, J=6.6 Hz), 6.6-6.8 (1H, m), 6.9-7.5 (5H, m), 7.97 (1H, s), 12.5 (1H, br); API-ES/MS: 372[M+H]⁺, 374[M+2+H]⁺, 394[M+Na]⁺, 396[M+2+Na]⁺.

### EXAMPLE 110

6-[6-Amino-5-chloro-2-(2-methoxyphenyl)-3-pyridyl]-2-isopropyl-3(2H)-pyridazinone was prepared in a similar manner to that of Example 81.

¹H NMR (DMSO-d₆, δ): 0.8-1.1 (6H, m), 3.33 (3H, s), 4.96 (1H, 7-plet, J=6.6 Hz), 6.62 (2H, br), 6.75 (1H, d, J=9.6 Hz), 6.8-7.4 (5H, m), 7.76 (1H, s);

API-ES/MS: 371[M+H]⁺, 393[M+Na]⁺, 395[M+2+Na]⁺.

### EXAMPLE 111

6-[6-Amino-5-chloro-2-(2-hydroxyphenyl)-3-pyridyl]-2-isopropyl-3(2H)-pyridazinone was prepared in a similar manner to that of Example 107.

¹H NMR (DMSO-d₆, δ): 0.99 (6H, d, J=6.6 Hz), 4.97 (1H, 7-plet, J=6.6 Hz), 6.6-6.9 (5H, m), 7.0-7.3 (3H, m), 7.77 (1H, s), 9.48 (1H, br); API-ES/MS: 357[M+H]⁺, 379[M+Na]⁺, 381[M+2+Na]⁺.

### EXAMPLE 112

2-Isopropyl-6-[2-(3-methoxyphenyl)-6-oxo-1,6-dihydro-3-pyridyl]-3(2H)-pyridazinone was prepared in a similar manner to that of Example 30.

¹H NMR (DMSO-d₆, δ): 1.06 (6H, d, J=6.6 Hz), 3.80 (3H, s), 5.03 (1H, 7-plet, J=6.6 Hz), 6.50 (1H, d, J=9.4 Hz), 6.72 (1H, d, J=9.5 Hz), 6.75-7.05 (4H, m), 7.2-7.4 (1H, m), 7.70 (1H, d, J=9.4 Hz), 11.9 (1H, br); API-ES/MS: 338[M+H]⁺, 360[M+Na]⁺.

### EXAMPLE 113

6-[5-Chloro-2-(3-methoxyphenyl)-6-oxo-1,6-dihydro-3-pyridyl]-2-isopropyl-3(2H)-pyridazinone was prepared in a similar manner to that of Example 34.

¹H NMR (DMSO-d₆, δ): 1.05 (6H, d, J=6.6 Hz), 3.71 (3H, s), 5.01 (1H, 7-plet, J=6.6 Hz), 6.7-7.1 (2H, m), 7.2-7.4 (1H, m), 7.99 (1H, s), 12.5 (1H, br); API-ES/MS: 372[M+H]⁺, 394[M+Na]⁺, 396[M+2+Na]⁺.

### EXAMPLE 114

6-[6-Amino-5-chloro-2-(3-methoxyphenyl)-3-pyridyl]-2-isopropyl-3(2H)-pyridazinone was prepared in a similar manner to that of Example 81.

¹H NMR (DMSO-d₆, δ): 1.07 (6H, d, J=6.6 Hz), 3.67 (3H, s), 5.04 (1H, 7-plet, J=6.6 Hz), 6.6-7.0 (6H, m), 7.05-7.35 (2H, m), 7.80 (1H, s); API-ES/MS: 371[M+H]⁺, 373[M+2+H]⁺, 393[M+Na]⁺, 395[M+2+Na]⁺.

### EXAMPLE 115

6-[6-Amino-5-chloro-2-(3-hydroxyphenyl)-3-pyridyl]-2-isopropyl-3(2H)-pyridazinone was prepared in a similar manner to that of Example 107.

¹H NMR(DMSO-d₆, δ): 1.10 (6H, d, J=6.6 Hz), 5.05 (1H, 7-plet, J=6.6 Hz), 6.5-6.8 (5H, m), 7.0-7.2 (3H, m), 7.77 (1H, s), 9.43 (1H, br); API-ES/MS: 357[M+H]⁺, 379[M+Na]⁺.

### EXAMPLE 116

6′-Amino-1-isopropyl-2′-phenyl-3,3′-bipyridin-6 (1H)-one was prepared in a similar manner to that of Example 81.

¹H NMR (DMSO-d₆, δ): 1.01 (6H, d, J=6.7 Hz), 4.91 (1H, d, J=6.7 Hz), 6.10 (2H, br), 6.2-6.3 (1H, m), 6.1-6.6 (1H, m), 7.1-7.5 (5H, m); API-ES/MS: 306[M+H]⁺, 328[M+Na]⁺.

### EXAMPLE 117

6′-Amino-S′-chloro-1-isopropyl-2′-phenyl-3,3′-bipyridin-6 (1H)-one was prepared in a similar manner to that of Example 34.

¹H NMR (DMSO-d₆, δ): 1.02 (6H, d, J=6.6 Hz), 4.91 (1H, m), 6.25 (1H, d, J=9.3 Hz), 6.46 (2H, br), 7.0-7.4 (7H, m), 7.69 (1H, s); API-ES/MS: 340[M+H]⁺, 342[M+2+H]⁺, 362[M+Na]⁺, 364[M+2+Na]⁺.

### EXAMPLE 118

A mixture of 1-isopropyl-2′-phenyl-3,3′-bipyridin-6,6′(1H,1′H)-dione (2.38 g) and N-chlorosuccinimide (2.28 g) in DMF (30 ml) was stirred at 50C. After 12 hour, aq.NaHCO₃ solution was added to the reaction mixture, which was extracted with EtOAc. The EtOAc phase was separated, dried over earth granular. The solvent was removed in vacuo to give oily residue. The residue was purified by silica gel column chromatography eluted with a mixture of CHCl₃ and MeOH. The fractions were concentrated in vacuo to obtain 5,5′-dichloro-1-isopropyl-2′-phenyl-3,3′-bipyridin-6,6′(1H,1′H)-dione (1.50 g) as white powder.

¹H NMR (DMSO-d₆, δ): 1.00 (6H, d, J=6.6 Hz), 4.90 (1H, 7-plet, J=6.6 Hz), 7.2-7.5 (5H, m), 7.5-7.6 (1H, m), 7.96 (1H, s), 12.4 (1H, br); API-ES/MS: 375[M+H]⁺, 397[M+Na]⁺, 399[M+2+Na]⁺.

### EXAMPLE 119

6′-Amino-5,5′-dichloro-1-isopropyl-2′-phenyl-3,3′-bipyridin-6 (1H)-one was prepared in a similar manner to that of Example 81.

¹H NMR (DMSO-d₆, δ): 1.03 (6H, d, J=6.6 Hz), 4.93 (1H, 7-plet, J=6.6 Hz), 6.51 (2H, br), 7.2-7.4 (5H, m), 7.45-7.6 (1H, m), 7.79 (1H, s); API-ES/MS: 375[M+H]⁺, 376[M+1+H]⁺, 396[M+Na]⁺, 398[M+2+Na]⁺.

### EXAMPLE 120

A mixture of 2-chloro-5-(1-isopropyl-6-oxo-1,6-dihydro-3-pyridazinyl)-6-phenylnicotinamide (5.0 g) and N,N-dimethylformamide-dimethoxyacetal (20 ml) was stirred at 100° C. After 12 hours, the solvent was removed in vacuo to give an oily residue. IPE was poured into the residue to give a pale yellow precipitate. The precipitate was collected by filtration to obtain 2-chloro-N-[(1E)-(dimethylamino)methylene]-5-(1-isopropyl-6-oxo-1,6-dihydro-3-pyridazinyl)-6-phenyl-nicotinamide (5.0 g) as white powder.

API-ES/MS: 423[M+H]⁺.

### EXAMPLE 121

A mixture of 6-[6-chloro-5-(1-methyl-1H-1,2,4-triazol-5-yl)-2-phenyl-3-pyridyl]-2-isopropyl-3 (2H)-pyridazinone (5.0 g) and methylhydrazine (5 ml) in EtOH (50 ml) was stirred at 80° C. After 12 hours, the solvent was removed in vacuo to give an oily residue. IPE was poured into the residue to give a pale yellow precipitate. The precipitate was collected by filtration to obtain 6-[6-chloro-5-(1-methyl-1H-1,2,4-triazol-5-yl)-2-phenyl-3-pyridyl]-2-isopropyl-3 (2H)-pyridazinone (3.0 g) as white powder.

API-ES/MS: 407[M+H].

### EXAMPLE 122

A mixture of 6-[6-chloro-5-(1-methyl-1H-1,2,4-triazol-5-yl)-2-phenyl-3-pyridyl]-2-isopropyl-3 (2H)-pyridazinone (60 mg) and 28% aq.ammonia (1 ml) in dioxane (1 ml) was heated in a sealed tube at 150° C. After 7 days, water and CHCl₃ were added to the reaction mixture. The organic layer was dried over earth granular. The solvent was removed in vacuo to give a precipitate. The precipitate was purified by silica gel column chromatography eluted with a mixture of CHCl₃ and MeOH. The fractions were concentrated in vacuo to obtain 6-[6-amino-5-(1-methyl-1H-1,2,4-triazol-5-yl)-2-phenyl-3-pyridyl]-2-isopropyl-3 (2H)-pyridazinone (40 mg) as pale yellow powder.

mp: 253-254° C.; IR (KBr): 3147, 1658, 1587 cm⁻¹; ¹H NMR (DMSO-d₆, δ): 1.02 (6H, d, J=6.6 Hz), 4.02 (3H, s), 5.02 (1H, 7-plet, J=6.6 Hz), 6.80 (1H, d, J=9.6 Hz), 7.06 (2H, br), 7.24 (1H, d, J=9.6 Hz), 7.3-7.5 (5H, m), 8.02 (1H, s), 8.14 (1H, s); API-ES/MS: 388[M+H]⁺, 410[M+Na]⁺; Elemental Analysis for C₁₀H₂₁N₇O; Calcd.: C, 64.21; H, 5.54; N, 24.96; Found : C, 64.41; H, 5.45; N, 24.63.

### EXAMPLE 123

A mixture of 2-(2-isopropyl-3 (2H)-pyridazinon-6-yl)-1-phenyl-2-propen-1-one (4.72 g) and methyl 3-aminocrotonate (2.47 g) in dimethylformamide (6 ml) was heated at 120° C. under stirring in a nitrogen stream for 7 hours. After cooling to ambient temperature, the reaction mixture was dissolved in EtOAc. The mixture was washed with NaHCO₃ solution and water successively. After drying over MgSO₄, the solvent was removed in vacuo to give crystalline mass, which was triturated in IPE, collected by filtration and dried to afford a mixture of methyl 2-methyl-5-(2-isopropyl-3 (2H)-pyridazinon-6-yl)-6-phenyl-pyridine-3-carboxylate and methyl 2-methyl-5-(2-isopropyl-3(2H)-pyridazinon-6-yl)-6-phenyl-1,4-dihydropyridine-3-carboxylate (about 2:7 mixture from NMR analysis) (5.00g) as yellow crystals. The filtrate was concentrated in vacuo to give a residue, which was subjected to column chromatography on silica gel eluting with a mixture of n-hexane and EtOAc (1:1). The fractions containing the oxidized pyridine derivative were combined and evaporated to give a crystal (437.9 mg) of methyl 2-methyl-5-(2-isopropyl-3 (2H)-pyridazinon-6-yl)-6-phenyl-pyridine-3-carboxylate as colorless crystal. From the second fractions, a mixture of pyridine and dihydro-pyridine derivatives (about 3:1 mixture from NMR analysis) was obtained (231.0 mg) as light yellow crystals. (The yield was calculated as the obtained product was all oxidized pyridine derivative.)

Methyl 2-methyl-5-(2-isopropyl-3 (2H)-pyridazinon-6-yl)-6-phenylpyridine-3-carboxylate

¹H NMR (CDCl₃, δ): 1.29 (6H, d, J=6.64 Hz), 2.95 (3H, s), 3.98 (3H, s), 5.31 (1H, 7-plet, J=6.64 Hz), 6.70 (1H, d, J=9.56 Hz), 6.83 (1H, d, J=9.56 Hz), 7.35-7.45 (5H, m), 8.41 (1H, s); API-ES/MS: 364[M+H]⁺, 386[M+Na]⁺.

Methyl 2-methyl-5-(2-isopropyl-3 (2H)-pyridazinon-6-yl)-6-phenyl-1,4-dihydropyridine-3-carboxylate

¹H NMR (CDCl₃, δ): 1.25 (6H, d, J=6.60 Hz), 2.30 (3H, s), 3.56 (2H, s), 3.74 (3H, s), 5.22 (1H, 7-plet, J=6.60 Hz), 5.41 (1H, s), 6.43 (1H, d, J=10.3 Hz), 6.50 (1H, d, J=10.3 Hz), 7.24-7.43 (5H, m); API-ES/MS: 366[M+H]⁺, 388[M+Na]⁺. API-ES, Negative/MS: 364[M−H]⁺.

### EXAMPLE 124

To a solution of methyl 2-methyl-5-(2-isopropyl-3(2H)-pyridazinon-6-yl)-6-phenyl-1,4-dihydropyridine-3-carboxylate (4.68 g) in EtOAc (150 ml) was added manganese(IV) oxide (11.1 g) under stirring at ambient temperature. The mixture was stirred for 3 hours under the same conditions and allowed to stand overnight. Undissolved mass was filtered off through Celite. The filtrate and washings were combined and evaporated in vacuo to give crystalline mass, which was triturated in IPE, collected by filtration and dried to afford pure methyl 2-methyl-5-(2-isopropyl-3(2H)-pyridazinon-6-yl)-6-phenylpyridine-3-carboxylate (4.26 g) as light yellow crystalline powder.

¹H NMR (CDCl₃, δ): 1.29 (6H, d, J=6.64 Hz), 2.95 (3H, s), 3.98 (3H, s), 5.31 (1H, 7-plet, J=6.64 Hz), 6.70 (1H, d, J=9.56 Hz), 6.83 (1H, d, J=9.56 Hz), 7.35-7.45 (5H, m), 8.41 (1H, s); API-ES/MS: 364[M+H]⁺, 386[M+Na]⁺.

### EXAMPLE 125

Ethyl 5-(1-isopropyl-6-oxo-1,6-dihydro-3-pyridazinyl)-2-methyl-6-phenylnicotinate was prepared in a similar manner to that of Example 123.

¹H NMR (DMSO-d₆, δ): 1.02 (6H, d, J=6.64 Hz), 1.35 (3H, t, J=7.08 Hz), 2.82 (3H, s), 4.37 (2H, q, J=7.08 Hz), 5.04 (1H, 7-plet, J=6.64 Hz), 6.88 (1H, d, J=9.60 Hz), 7.34 (1H, d, J=9.60 Hz), 7.35-7.39 (5H, m), 8.35 (1H, s); API-ES/MS: 378[M+H]⁺, 400[M+Na]⁺.

### EXAMPLE 126

To a solution of methyl 2-methyl-5-(2-isopropyl-3 (2H)-pyridazinon-6-yl)-6-phenylpyridine-3-carboxylate (0.97 g) in DME (20 ml) was added 1N aq.NaOH solution (5.34 ml) under stirring at ambient temperature. The stirring was continued for 1.5 hours under the same conditions. The organic solvent was removed in vacuo and to the resultant aqueous residue was added water. 1N Aq.HCl solution (5.35 ml) was added to the mixture gradually under stirring. The resultant precipitate was collected by filtration, washed with water and dried to give 2-methyl-5-(2-isopropyl-3 (2H)-pyridazinon-6-yl)-6-phenylpyridine-3-carboxylic acid (933 mg) as white powder.

¹H NMR (DMSO-d₆, δ): 1.03 (6H, d, J=6.62 Hz), 2.83 (3H, s), 5.05 (1H, 7-plet, J=6.62 Hz), 6.86 (1H, d, J=9.56 Hz), 7.32 (1H, d, J=9.56 Hz), 7.38 (5H, s), 8.35 (1H, s), 13.3-13.7 (1H, br, s); API-ES/MS: 350[M+H]⁺, 372[M+Na]⁺.

### EXAMPLE 127

A solution of 2-methyl-5-(2-isopropyl-3 (2H)-pyridazinon-6-yl)-6-phenylpyridine-3-carboxylic acid (698.8 mg), diphenylphosphoryl azide (0.431 ml) and Et₃N (0.279 ml) in dry t-butyl alcohol (8 ml) was heated at 80° C. for 15 minutes and at 90° C. for 3.5 hours. After cooling to ambient temperature, t-butyl alcohol was removed in vacuo to give a residue, to which were added EtOAc and aq.NaHCO₃ solution under stirring. The separated organic layer was washed with aq.NaHCO₃ solution, water twice and brine. After drying over MgSO₄, the solvent was evaporated to a half of volume in vacuo to afford precipitate, which was collected by filtration, washed with IPE and dried to give N,N′-bis[2-methyl-5-(2-isopropyl-3 (2H)-pyridazinon-6-yl)-6-phenylpyridin-3-yl]urea (371.1 mg) as white crystalline powder. The filtrate and the washings were combined and evaporated in vacuo to afford a residue, which was subjected to column chromatography on silica gel eluting with a mixture of CHCl₃ and MeOH (50:1). The fractions containing the desired product were combined and evaporated in vacuo to give an amorphous mass, which was triturated in IPE to afford crystalline powder. Collection by filtration, washing with IPE and dryness gave pure 2-methyl-3-t-butoxycarbonylamino-5-(2-isopropyl-3 (2H)-pyridazinon-6-yl)-6-phenylpyridine (227.5 mg).

2-Methyl-3-t-butoxycarbonylamino-5-(2-isopropyl-3 (2H)-pyridazinon-6-yl)-6-phenylpyridine

¹H NMR (CDCl₃, δ): 1.23 (6H, d, J=6.70 Hz), 1.56 (9H, s), 2.61 (3H, s), 5.26 (1H, 7-plet, J=6.70 Hz), 6.42 (1H, s), 6.70 (1H, d, J=9.54 Hz), 6.92 (1H, d, J=9.54 Hz), 7.27-7.36 (5H, m), 8.41 (1H, s); API-ES/MS: 421[M+H]⁺, 443[M+Na]⁺.

N,N′-bis [2-Methyl-5-(2-isopropyl-3 (2H)-pyridazinon-6-yl)-6-phenylpyridin-3-yl]urea

¹H NMR (CDCl₃, δ): 1.25 (12H, d, J=6.66 Hz), 2.60 (6H, s), 5.30 (2H, 7-plet, J=6.66 Hz), 6.71 (2H, d, J=9.54 Hz), 6.93 (2H, d, J=9.54 Hz), 7.29-7.37 (10H, m), 7.94 (2H, s), 8.50 (2H, s); API-ES/MS: 667[M+H]⁺, 689[M+Na]⁺; API-ES, Negative/MS: 665[M−H]⁺.

### EXAMPLE 128

A mixture of 2-methyl-3-t-butoxycarbonylamino-5-(2-isopropyl-3 (2H)-pyridazinon-6-yl)-6-phenylpyridine (200 mg) and 4N HCl/EtOAc (2 ml) in EtOAc (2 ml) was stirred for 1.5 hours at ambient temperature to afford white precipitate. To the reaction mixture was added IPE and the resultant precipitate was collected by filtration. The obtained powder was dissolved in water and the aqueous solution was made basic with a saturated aq.NaHCO₃ solution. The mixture was extracted with EtOAc, washed with water twice and dried over Na₂SO₄. Removal of the solvent gave an amorphous mass, which was crystallized by trituration in IPE. Collection by filtration, washing with IPE and drying gave 2-methyl-3-amino-5-(2-isopropyl-3 (2H)-pyridazinon-6-yl)-6-phenylpyridine (129.9 mg).

¹H NMR (DMSO-d₆, δ) 1.11 (6H, d, J=6.62 Hz), 2.37 (3H, s), 5.08 (1H, 7-plet, J=6.62 Hz), 5.38 (2H, s), 6.76 (1H, d, J=9.58 Hz), 7.06 (1H, d, J=9.58 Hz), 7.09 (1H, s), 7.17-7.33 (5H, m); API-ES/MS: 321[M+H]⁺, 343[M+Na]⁺.

### EXAMPLE 129

To a solution of methyl 2-methyl-5-(2-isopropyl-3 (2H)-pyridazinon-6-yl)-6-phenylpyridine-3-carboxylate (363.4 mg) in carbon tetrachloride (15 ml) was added N-bromosuccinimide (445.0 mg) under stirring. To the mixture was added a catalytic amount of benzoyl peroxide. The mixture was refluxed under stirring for 5 hours. After cooling to ambient temperature, undissolved mass was filtered off. The filtrate and the washings were combined and evaporated in vacuo to afford an oil, which was subjected to column chromatography on silica gel eluting with CHCl₃. The fractions containing the desired product were combined and evaporated in vacuo to afford crude methyl 2-bromomethyl-5-(2-isopropyl-3 (2H)-pyridazinon-6-yl)-6-phenylpyridine-3-carboxylate (278.5 mg) as an amorphous mass, which was used in a following reaction without further purification.

¹H NMR (CDCl₃, δ): 1.30 (6H, d, J=6.58 Hz), 4.03 (3H, s), 5.13 (2H, s), 5.32 (1H, 7-plet, J=6.58 Hz), 6.71 (1H, d, J=9.62 Hz), 6.85 (1H, d, J=9.62 Hz), 7.35-7.49 (5H, m), 8.47 (1H, s).

### EXAMPLE 130

To a solution of methyl 2-bromomethyl-5-(2-isopropyl-3 (2H)-pyridazinon-6-yl)-6-phenylpyridine-3-carboxylate (278.5 mg) in DMF (3 ml) was added potassium phthalimide (117 mg). The mixture was heated at 100° C. under stirring for 2.5 hours. The reaction mixture was poured into water and extracted with EtOAc. The extract was washed with water twice, dried over MgSO₄ and evaporated in vacuo to afford an oil, which was crystallized by trituration in EtOAc. The resultant crystalline powder was collected by filtration, washed with a mixture of EtOAc and IPE and dried to give methyl 2-phthalimidomethyl-5-(2-isopropyl-3 (2H)-pyridazinon-6-yl)-6-phenylpyridine-3-carboxylate (157.9 mg) as crystalline powder.

¹H NMR (DMSO-d₆, δ): 0.98 (6H, d, J=6.62 Hz), 3.98 (3H, s), 5.02 (1H, 7-plet, J=6.62 Hz), 5.39 (2H, s), 6.89 (1H, d, J=9.58 Hz), 6.97-7.22 (5H, m), 7.39 (1H, d, J=9.58 Hz), 7.89-7.98 (4H, m), 8.48 (1H, s); API-ES/MS: 509[M+H]⁺, 531[M+Na]⁺.

### EXAMPLE 131

To a suspension of methyl 2-phthalimidomethyl-5-(2-isopropyl-3(2H)-pyridazinon-6-yl)-6-phenylpyridine-3-carboxylate (153.0 mg) in acetonitrile (5 ml) was added trimethylsilyl iodide (0.085 ml) under stirring at ambient temperature. The resultant red solution was refluxed for 24 hours. An additional trimethylsilyl iodide (0.1 ml) was added and the mixture was refluxed for 7 hours further. A further additional trimethylsilyl iodide (0.1 ml) was added to the reaction mixture, which was further refluxed for additional 24 hours. The reaction mixture was poured into water and extracted with EtOAc. The extract was washed with water twice and dried over MgSO₄. Removal of the solvent in vacuo gave a red oil, which was subjected to preparative thin layer chromatography on silica gel developing with a mixture of CHCl₃ and MeOH (15:1) to afford an amorphous mass. This was crystallized by trituration in IPE and collected by filtration to give pure 2-phthalimidomethyl-5-(2-isopropyl-3 (2H)-pyridazinon-6-yl)-6-phenylpyridine-3-carboxylic acid (64.6 mg).

¹H NMR (DMSO-d₆, δ): 0.99 (6H, d, J=6.63 Hz), 5.02 (1H, 7-plet, J=6.63 Hz), 5.39 (2H, s), 6.87 (1H, d, J=9.62 Hz), 6.97-7.25 (5H, m), 7.37 (1H, d, J=9.62 Hz), 7.86-7.98 (4H, m), 8.46 (1H, s), 13.7-14.1 (1H, br, s); API-ES, Negative/MS: 493[M−H]⁺.

### EXAMPLE 132

Methyl 2-dimethoxymethyl-5-(2-isopropyl-3(2H)-pyridazinon-6-yl)-6-phenylpyridine-3-carboxylate was prepared in a similar manner to that of Example 124.

¹H NMR (CDCl₃, δ): 1.32 (6H, d, J=6.68 Hz), 3.55 (6H, s), 3.99 (3H, s), 5.32 (1H, 7-plet, J=6.68 Hz), 6.05 (1H, s), 6.68 (1H, d, J=9.54 Hz), 6.80 (1H, d, J=9.54 Hz), 7.32-7.49 (5H, m), 8.25 (1H, s); API-ES/MS: 424[M+H]⁺, 446[M+Na]⁺.

### EXAMPLE 133

To a solution of methyl 2-dimethoxymethyl-5-(2-isopropyl-3(2H)-pyridazinon-6-yl)-6-phenylpyridine-3-carboxylate (2.0 g) in acetone (20 ml) was added 6N aq.HCl solution (2 ml) under stirring at ambient temperature. The mixture was stirred for 2 hours under the same temperature. Acetone was removed in vacuo to give a residue, which was dissolved in a mixture of EtOAc and aq.NaHCO₃ solution under stirring. The separated organic layer was washed with water twice, dried over MgSO₄ and evaporated to afford an orange oil, which was triturated in IPE. The resultant crystalline powder was collected by filtration, washed with IPE and dried to give methyl 2-formyl-5-(2-isopropyl-3 (2H)-pyridazinon-6-yl)-6-phenylpyridine-3-carboxylate (1.37 g).

¹H NMR (CDCl₃, δ): 1.33 (6H, d, J=6.58 Hz), 4.03 (3H, s), 5.33 (1H, 7-plet, J=6.58 Hz), 6.71 (1H, d, J=9.58 Hz), 6.83 (1H, d, J=9.58 Hz), 7.37-7.52 (5H, m), 8.28 (1H, s), 10.34 (1H, s); API-ES/MS: 378[M+H]⁺, 400[M+Na]⁺.

### EXAMPLE 134

To a solution of methyl 2-formyl-5-(2-isopropyl-3(2H)-pyridazinon-6-yl)-6-phenyl-3-carboxylate (1.0 g) were added hydroxylamine hydrochloride (221 mg) and NaOAc (261 mg) under stirring at ambient temperature. The stirring was continued for 1 hour under the same conditions. To the reaction mixture was added acetic anhydride (0.33 ml). The mixture was heated at 100° C. under stirring for 3 hours. AcOH was removed in vacuo and water and aq.NaHCO₃ solution were added to the residue under stirring.

The resultant yellow precipitate was collected by filtration, washed with water and dried to give methyl 2-cyano-5-(2-isopropyl-3(2H)-pyridazinon-6-yl)-6-phenyl-3-carboxylate (759.1 mg).

¹H NMR (DMSO-d₆, δ): 1.00 (6H, d, J=6.58 Hz), 3.99 (3H, s), 5.04 (1H, 7-plet, J=6.58 Hz), 6.93 (1H, d, J=9.66 Hz), 7.38-7.47 (6H, m), 8.64 (1H, s); API-ES/MS: 375[M+H]⁺, 397[M+Na]⁺.

### EXAMPLE 135

To a solution of methyl 2-formyl-5-(2-isopropyl-3 (2H)-pyridazinon-6-yl)-6-phenylpyridine-3-carboxylate (200 mg) in THF (10 ml) was added LiBH₄ (23.3 mg) under stirring and cooling in an ice-bath. The mixture was stirred at ambient temperature for 3 hours and an additional LiBH₄ (23.3 mg) was added thereto. The resultant mixture was stirred at ambient temperature overnight. THF was removed in vacuo to give a residue, to which was added a mixture of EtOAc and brine under stirring. The separated organic layer was washed with brine twice and dried over MgSO₄. Removal of the solvent afforded on oil, which was subjected to column chromatography on silica gel eluting with a mixture of EtOAc and CHCl₃ (1:4). From the first fraction 3-(2-isopropyl-3 (2H)-pyridazinon-6-yl)-2-phenyl-4,7-dihydrofuro[3,4-b]pyridin-5 (1H)-one (65.6 mg) was obtained. The fractions containing the desired product were combined and evaporated in vacuo to give an amorphous mass of 2,3-dihydroxymethyl-5-(2-isopropyl-3 (2H)-pyridazinon-6-yl)-6-phenyl-pyridine (65.2 mg), which was triturated in IPE to afford crystalline powder.

2,3-Dihydroxymethyl-5-(2-isopropyl-3 (2H)-pyridazinon-6-yl)-6-phenylpyridine

¹H NMR (DMSO-d₆, δ): 1.05 (6H, d, J=6.58 Hz), 4.68 (2H, d, J=5.56 Hz), 4.75 (2H, d, J=5.36 Hz), 5.06 (1H, 7-plet, J=6.58 Hz), 5.19 (1H, t, J=5.56 Hz), 5.39 (1H, t, J=5.36 Hz), 6.85 (1H, d, J=9.56 Hz), 7.25 (1H, d, J=9.56 Hz), 7.32-7.43 (5H, m), 8.01 (1H, s); API-ES/MS: 352[M+H]⁺, 374[M+Na]⁺.

3-(2-Isopropyl-3 (2H)-pyridazinon-6-yl)-2-phenyl-4,7-dihydrofuro[3,4-b]pyridin-5(1H)-one

¹H NMR (DMSO-d₆, δ): 1.11 (6H, d, J=6.56 Hz), 3.41 (2H, s), 4.76 (2H, s), 5.02 (1H, 7-plet, J=6.56 Hz), 6.52 (1H, d, J=9.70 Hz), 6.67 (1H, d, J=9.70 Hz), 7.23-7.43 (5H, m), 9.31 (1H, s); API-ES/MS: 350[M+H]⁺, 372[M+Na]⁺.

### EXAMPLE 136

A mixture of methyl 2-formyl-5-(2-isopropyl-3 (2H)-pyridazinon-6-yl)-6-phenylpyridine-3-carboxylate (100 mg), N-methylhydroxylamine hydrochloride (26.6 mg) and pyridine (27.2 mg) in EtOH (10 ml) was refluxed under stirring for 7 hours. EtOH was removed in vacuo to afford a residue, which was dissolved in a mixture of EtOAc and aq.NaHCO₃ solution under stirring. The separated organic layer was washed with water twice and dried over MgSO₄. Removal of the solvent gave crude 3-methoxycarbonyl-5-(2-isopropyl-3 (2H)-pyridazinon-6-yl)-6-phenylpyridine-2-methyliminomethyl-N-oxide as an amorphous mass (111.5 mg), which was used in a following reaction without further purification.

### EXAMPLE 137

A mixture of crude 3-methoxycarbonyl-5-(2-isopropyl-3 (2H)-pyridazinon-6-yl)-6-phenylpyridin-2-methyliminomethyl-N-oxide (111.5 mg), acetic anhydride (2.5 ml) and AcOH (0.06 ml) was refluxed under stirring for 1.5 hours. After cooling to ambient temperature, the reaction mixture was made basic with a saturated aq.NaHCO₃ solution under stirring. The resultant aqueous mixture was extracted with EtOAc twice and washed with water three times. After drying over MgSO₄, the solvent was removed in vacuo to give a residue, which was subjected to preparative thin layer chromatography on silica gel developing with a mixture of CHCl3 and EtOAc (2:1). The obtained amorphous mass (about 90 mg) was treated with a mixture of MeOH and 6N aq.HCl solution (4:1/5 ml) overnight. After removal of MeOH in vacuo, the residual mixture was adjusted to pH 8 with aq.NaHCO₃ solution and extracted with EtOAc. The extract was washed with water three times and dried over MgSO₄. Removal of the solvent in vacuo gave an oil (59.7 mg), which was triturated in IPE to afford crystalline powder. Collection by filtration, washing with IPE and drying afforded methyl 2-methylaminocarbonyl-5-(2-isopropyl-3 (2H)-pyridazinon-6-yl)-6-phenylpyridin-3-carboxylate (19.7 mg). ¹H NMR (DMSO-d₆, δ): 0.90 (6H, d, J=6.58 Hz), 3.13 (3H, s), 5.03 (1H, 7-plet, J=6.58 Hz), 6.92 (1H, d, J=9.62 Hz), 7.38-7.47 (6H, m), 8.51 (1H, s); API-ES/MS: 407[M+H]⁺, 429[M+Na]⁺.

### EXAMPLE 138

To a solution of triethyl phosphonoacetate (253 mg) in THF (5 ml) was added NaH (60% suspension in mineral oil; 45.2 mg) under stirring and cooling in an ice-bath. The mixture was stirred for 0.5 hour at ambient temperature. To the mixture obtained above was added dropwise a solution of methyl 2-formyl-5-(2-isopropyl-3 (2H)-pyridazinon-6-yl)-6-phenyl-pyridine-3-carboxylate (377.4 mg) in THF (3 ml) under cooling in an ice-bath. After the completion of the addition, the mixture was stirred for 2 hours at ambient temperature. THF was removed in vacuo to give a residue, which was dissolved in a mixture of EtOAc, aq.NaHCO₃ solution and water under stirring. The separated organic layer was washed with water twice and dried over MgSO₄. Removal of the solvent afforded an oil (0.48 g), which was subjected to column chromatography on silica gel eluting with CHCl₃. The fractions containing the desired product were combined and evaporated in vacuo to give methyl 2-[(1E)-3-ethoxy-3-oxo-1-propen-1-yl]-5-(2-isopropyl-3 (2H)-pyridazinon-6-yl)-6-phenyl-pyridine-3-carboxylate (419.2 mg) as colorless crystals.

¹H NMR (DMSO-d₆, δ): 1.01 (6H, d, J=6.60 Hz), 1.28 (3H, t, J=7.12 Hz), 3.95 (3H, s), 4.23 (2H, q, J=7.12 Hz), 5.04 (1H, 7-plet, J=6.60 Hz), 6.91 (1H, d, J=9.66 Hz), 7.10 (1H, d, J=15.36 Hz), 7.39-7.48 (6H, m), 8.47 (1H, s), 8.48 (1H, d, J=15.36 Hz); API-ES/MS: 448[M+H]⁺, 470[M+Na]⁺.

The following compound(s) was (were) obtained in a similar manner to that of Example 126.

### EXAMPLE 139

2-(Dimethoxymethyl)-5-(2-isopropyl-3 (2H)-pyridazinon-6-yl)-6-phenylpyridine-3-carboxylic acid was prepared in a similar manner to that of Example 126.

¹H NMR (DMSO-d₆, δ): 1.02 (6H, d, J=6.58 Hz), 3.42 (6H, s), 5.04 (1H, 7-plet, J=6.58 Hz), 5.98 (1H, s), 6.88 (1H, d, J=9.56 Hz), 7.37 (1H, d, J=9.56 Hz), 7.39 (5H, s), 8.27 (1H, s), 13.5-13.7 (1H, br, s); API-ES, Negative/MS: 408[M−H]⁺.

### EXAMPLE 140

2-(Dimethoxymethyl)-3-ethoxycarbonylamino-5-(2-isopropyl-3(2H)-pyridazinon-6-yl)-6-phenylpyridine was prepared in a similar manner to that of Example 127.

¹H NMR (CDCl₃, δ): 1.24 (6H, d, J=6.70 Hz), 1.35 (3H, t, J=7.20 Hz), 3.55 (6H, s), 4.25 (2H, q, J=7.20 Hz), 5.27 (1H, 7-plet, J=6.70 Hz), 5.42 (1H, s), 6.71 (1H, d, J=9.58 Hz), 6.91 (1H, d, J=9.58 Hz), 7.29-7.37 (5H, m), 8.44 (1H, s), 8.81 (1H, s); API-ES/MS: 453[M+H]⁺, 475[M+Na]⁺.

### EXAMPLE 141

2-Formyl-3-ethoxycarbonylamino-5-(2-isopropyl-3 (2H)-pyridazinon-6-yl)-6-phenylpyridine was prepared in a similar manner to that of Example 133.

¹H NMR (CDCl₃, δ): 1.24 (6H, d, J=6.72 Hz), 1.37 (3H, t, J=7.05 Hz), 4.30 (2H, q, J=7.05 Hz), 5.28 (1H, 7-plet, J=6.72 Hz), 6.75 (1H, d, J=9.54 Hz), 6.98 (1H, d, J=9.54 Hz), 7.34-7.46 (5H, m), 9.08 (1H, s), 10.18 (1H, s), 10.42 (1H, s); API-ES, Negative/MS: 405[M−H]⁺.

### EXAMPLE 142

To a solution of 2-formyl-3-ethoxycarbonylamino-5-(2-isopropyl-3 (2H)-pyridazinon-6-yl)-6-phenylpyridine (101.6 mg) and 1,1-dimethylpropylenediamine (30.7 mg) in 1,2-dichloroethane (2.5 ml) were added NaBH(OAc)₃ (79.5 mg) and a catalytic amount of ACOH under stirring at ambient temperature. The stirring was continued for 5.5 hours under the same conditions. After removal of the solvent, an aq.NaHCO₃ solution was added to the residue. The mixture was extracted with EtOAc, washed with water twice and dried over MgSO₄. Removal of the solvent in vacuo gave an oil, which was subjected to preparative thin layer chromatography on silica gel developing with a mixture of CHCl₃ and MeOH (10:1) to afford the desired 2-dimethylaminopropylaminomethyl-3-ethoxycarbonylamino-5-(2-isopropyl-3(2H)-pyridazinon-6-yl)-6-phenylpyridine (25.7 mg) as an oil. This was triturated in IPE to give white powder of the product (24.1 mg).

¹H NMR (DMSO-d₆, δ): 1.07 (6H, d, J=6.60 Hz), 1.26 (3H, t, J=7.04 Hz), 1.60 (2H, 5-plet, J=6.72 Hz), 2.10 (6H, s), 2.28 (2H, t, J=6.72 Hz), 2.57 (2H, t, J=6.72 Hz), 4.08 (2H, s), 4.17 (2H, q, J=7.04 Hz), 5.07 (1H, 7-plet, J=6.60 Hz), 6.83 (1H, d, J=9.60 Hz), 7.20 (1H, d, J=9.60 Hz), 7.23-7.40 (5H, m), 8.45 (1H, s); API-ES/MS: 493[M+H]⁺.

### EXAMPLE 143

2-Pyridylmethylaminomethyl-3-ethoxycarbonylamino-5-(2-isopropyl-3 (2H)-pyridazinon-6-yl)-6-phenylpyridine was prepared in a similar manner to that of Example 142.

¹H NMR (CDCl₃, δ): 1.24 (6H, d, J=6.70 Hz), 1.37 (3H, t, J=7.08 Hz), 4.02 (2H, s), 4.20 (2H, s), 4.28 (2H, q, J=7.08 Hz), 5.27 (1H, 7-plet, J=6.70 Hz), 6.71 (1H, d, J=9.54 Hz), 6.92 (1H, d, J=9.54 Hz), 7.19-7.36 (8H, m), 7.63-7.73 (1H, m), 8.61-8.66 (2H, m), 10.56 (1H, s); API-ES/MS: 499[M+H]⁺.

### EXAMPLE 144

2-Methoxyethylaminomethyl-3-ethoxycarbonylamino-5-(2-isopropyl-3 (2H)-pyridazinon-6-yl)-6-phenylpyridine was prepared in a similar manner to that of Example 142.

¹H NMR (CDCl₃, δ): 1.24 (6H, d, J=6.62 Hz), 1.35 (3H, t, J=7.08 Hz), 2.86 (2H, t, J=5.12 Hz), 3.39 (3H, s), 3.56 (3H, t, J=5.12 Hz), 4.22 (2H, s), 4.26 (2H, q, J=7.08 Hz), 5.27 (1H, 7-plet, J=6.62 Hz), 6.70 (1H, d, J=9.54 Hz), 6.90 (1H, d, J=9.54 Hz), 7.29-7.35 (5H, m), 8.61 (1H, s), 10.53 (1H, s); API-ES/MS: 466[M+H]⁺.

### EXAMPLE 145

2-Phenoxyethylaminomethyl-3-ethoxycarbonylamino-5-(2-isopropyl-3 (2H)-pyridazinon-6-yl)-6-phenylpyridine was prepared in a similar manner to that of Example 142.

¹H NMR (CDCl₃, δ): 1.25 (6H, d, J=6.68 Hz), 1.30 (3H, t, J=7.08 Hz), 3.10 (2H, t, J=4.98 Hz), 4.13 (3H, t, J=4.98 Hz), 4.23 (2H, q, J=7.08 Hz), 4.29 (2H, s), 5.27 (1H, 7-plet, J=6.68 Hz), 6.70 (1H, d, J=9.54 Hz), 6.77-7.01 (4H, m), 7.25-7.40 (7H, m), 8.61 (1H, s), 10.42 (1H, s); API-ES/MS: 528[M+H]⁺.

### EXAMPLE 146

To a solution of 2-formyl-3-ethoxycarbonylamino-5-(2-isopropyl-3 (2H)-pyridazinon-6-yl)-6-phenylpyridine (1.19 g) in ACOH (20 ml) was added NaBH(OAc)₃ (1.24 g) portionwise under stirring at ambient temperature. After the addition was completed, the mixture was stirred for 1 hour under the same conditions. An additional NaBH(OAc)₃ (0.31 g) was added to the reaction mixture and stirred for 1 hour further. AcOH was removed in vacuo to give a residue, to which was added water. The mixture was made basic with a saturated aq.NaHCO₃ solution under stirring. Crystalline mass was obtained by addition of a small amount of EtOAc and collected by filtration to afford the first crop of the desired product. The filtrate was extracted with EtOAc and washed with water twice. After drying over MgSO₄, the solvent was removed in vacuo to give an oil containing the desired product, which was crystallized by tritulation in IPE and collected by filtration to afford the second crop of the desired product. The combined product was washed with IPE, collected by filtration, washed with IPE and dried to give pure 2-hydroxymethyl-3-ethoxycarbonylamino-5-(2-isopropyl-3(2H)-pyridazinon-6-yl)-6-phenylpyridine (1.14 g) as light yellow crystalline powder.

¹H NMR (DMSO-d₆, δ): 1.06 (6H, d, J=6.56 Hz), 1.27 (3H, t, J=7.06 Hz), 4.18 (2H, q, J=7.06 Hz), 4.78 (2H, s), 5.07 (1H, 7-plet, J=6.56 Hz), 5.6-5.9 (1H, br,), 6.84 (1H, d, J=9.64 Hz), 7.22 (1H, d, J=9.64 Hz), 7.27-7.40 (5H, m), 8.35 (1H, s), 9.0-9.3 (1H, br, s); API-ES/MS: 409[M+H]⁺, 431[M+Na]⁺.

### EXAMPLE 147

To a suspension of 2-hydroxymethyl-3-ethoxycarbonylamino-5-(2-isopropyl-3 (2H)-pyridazinon-6-yl)-6-phenylpyridine (513.1 mg) in 1,2-dichloroethane (5.5 ml) was added thionyl chloride (0.14 ml) under stirring at ambient temperature. The yellow clear solution was refluxed for 1 hour. 1,2-Dichloroethane was removed in vacuo to give a residue, which was dissolved in EtOAc, washed with an aq.NaHCO₃ solution and water twice and dried over MgSO₄. Removal of the solvent afforded an amorphous mass, which was crystallized by trituration in IPE and collected by filtration to give2-chloromethyl-3-ethoxycarbonylamino-5-(2-isopropyl-3 (2H)-pyridazinon-6-yl)-6-phenylpyridine (481.7 mg) as light yellow crystalline powder.

¹H NMR (CDCl₃, δ): 1.26 (6H, d, J=6.56 Hz), 1.38 (3H, t, J=7.06 Hz), 4.31 (2H, q, J=7.06 Hz), 4.86 (2H, s), 5.28 (1H, 7-plet, J=6.56 Hz), 6.71 (1H, d, J=9.64 Hz), 6.89 (1H, d, J=9.64 Hz), 7.11 (1H, br, s), 7.31-7.49 (5H, m), 8.54 (1H, s); API-ES/MS: 427[M+H]⁺, 449[M+Na]⁺.

### EXAMPLE 148

To a solution of 2-hydroxymethyl-3-ethoxycarbonylamino-5-(2-isopropyl-3 (2H)-pyridazinon-6-yl)-6-phenylpyridine (470.7 mg) in pyridine (5 ml) was added acetic anhydride (0.544 ml) under stirring and cooling in an ice-bath. The mixture was stirred at ambient temperature for 2 hours and allowed to stand overnight. To the reaction mixture was added MeOH under cooling and the solvent was removed in vacuo to give a residue, which was dissolved in EtOAc and washed with water three times. After drying over MgSO₄, the solvent was removed in vacuo to afford an amorphous mass (0.55 g), which was sonicated in IPE and the resultant white powder was collected by filtration, washed with IPE and dried to give pure 2-acetoxymethyl-3-ethoxycarbonylamino-5-(2-isopropyl-3 (2H)-pyridazinon-6-yl)-6-phenylpyridine (476.9 mg).

¹H NMR (CDCl₃, δ): 1.24 (6H, d, J=6.64 Hz), 1.37 (3H, t, J=7.17 Hz), 2.16 (3H, s), 4.29 (2H, q, J=7.17 Hz), 5.27 (1H, 7-plet, J=6.64 Hz), 5.35 (2H, s), 6.71 (1H, d, J=9.64 Hz), 6.90 (1H, d, J=9.64 Hz), 7.30-7.39 (5H, m), 8.32 (1H, s), 8.54 (1H, s); API-ES/MS: 451[M+H]⁺, 473[M+Na]⁺.

### EXAMPLE 149

A mixture of 2-formyl-3-ethoxycarbonylamino-5-(2-isopropyl-3(2H)-pyridazinon-6-yl)-6-phenylpyridine (406.5 mg), hydroxylamine hydrochloride (90.4 mg) and NaOAc (115.0 mg) in AcOH (10 ml)was stirred at ambient temperature for 1 hour. To the reaction mixture was added acetic anhydride (0.189 ml) and stirred at 100° C. for 5 hours. The solvent AcOH was removed in vacuo to give a residue, which was dissolved in a mixture of EtOAc and water. The separated organic layer was washed with water twice and dried over MgSO₄. Removal of the solvent afforded an amorphous mass (0.42 g), which was subjected to column chromatography on silica gel eluting with a mixture of CHCl₃ and MeOH (100:1). The fractions first eluted were combined and evaporated in vacuo to give an oil (99.3 mg), which crystallized by trituration in IPE and was collected by filtration to give 2-acetoxyiminomethyl-3-ethoxycarbonylamino-5-(2-isopropyl-3(2H)-pyridazinon-6-yl)-6-phenylpyridine as a light yellow crystals (54.5 mg). The second eluted fractions containing the intermediate above and desired product were combined and evaporated in vacuo to afford an amorphous mass, which was powdered by tritulation in IPE and collected by filtration to give light yellow crystallin powder as a mixture (150 mg). This was heated at 115° C. for 3 hours and at 140° C. for 2 hours under stirring in AcOH (1.0 ml) in the presence of a catalytic amount of NaOAc. The mixture was evaporated in vacuo to give a residue, to which was added water under stirring. The resultant precipitate was collected by filtration, washed with water and dried under reduced pressure to give 2-cyano-3-ethoxycarbonylamino-5-(2-isopropyl-3(2H)-pyridazinon-6-yl)-6-phenylpyridine (125.5 mg).

2-Cyano-3-ethoxycarbonylamino-5-(2-isopropyl-3 (2H)-pyridazinon-6-yl)-6-phenylpyridine

¹H NMR (DMSO-d₆, δ): 1.02 (6H, d, J=6.60 Hz), 1.29 (3H, t, J=7.12 Hz), 4.21 (2H, q, J=7.12 Hz), 5.04 (1H, 7-plet, J=6.60 Hz), 6.90 (1H, d, J=9.64 Hz), 7.31 (1H, d, J=9.64 Hz), 7.33-7.42 (5H, m), 8.26 (1H, s), 10.15-10.25 (1H, br, s); API-ES/MS: 404[M+H]⁺, 426[M+Na]⁺.

2-Acetoxyiminomethyl-3-ethoxycarbonylamino-5-(2-isopropyl-3(2H)-pyridazinon-6-yl)-6-phenylpyridine

¹H NMR (CDCl₃, δ): 1.23 (6H, d, J=6.58 Hz), 1.38 (3H, t, J=7.06 Hz), 2.29 (3H, s), 3.80 (2H, q, J=7.06 Hz), 5.27 (1H, 7-plet, J=6.58 Hz), 6.74 (1H, d, J=9.64 Hz), 6.98 (1H, d, J=9.64 Hz), 7.35-7.39 (5H, m), 8.70 (1H, s), 9.08 (1H, s), 10.20 (1H, s); API-ES/MS: 464[M+H]⁺, 486[M+Na]⁺; API-ES, Negative/MS: 462[M−H]⁺.

### EXAMPLE 150

To a suspension of 2-formyl-3-ethoxycarbonylamino-5-(2-isopropyl-3 (2H)-pyridazinon-6-yl)-6-phenylpyridine (406.4 mg) and dimethyl malonate (145.3 mg) in MeOH (9 ml) was added 2 drops of piperidine. The clear solution was refluxed for 4 hours. An additional dimethyl malonate (26.4 mg) and 1 drop of piperidine were added thereto. Reflux was continued for 2 hours further. After cooling to ambient temperature, MeOH was removed in vacuo to afford a residue, to which was added a mixture of EtOAc and water under stirring. The separated organic layer was washed with water twice and dried over MgSO₄. Removal of the solvent gave an oil, which was subjected to preparative thin layer chromatography on silica gel developing with a mixture of n-hexane and EtOAc (1:2) to afford ethyl

2-(2,2-dimethoxycarbonylvinyl)-5-(1-isopropyl-6-oxo-1,6-dihydro-3-pyridazinyl)-6-phenyl-3-pyridylcarbamate (193.2 mg) as yellow amorphous mass.

¹H NMR (CDCl₃, δ): 1.26 (6H, d, J=6.6 Hz), 1.37 (3H, t, J=7.1 Hz), 3.48 (3H, s), 3.88 (3H, s), 4.30 (2H, q, J=7.1 Hz), 5.28 (1H, 7-plet, J=6.6 Hz), 6.69 (1H, d, J=9.6 Hz), 6.86 (1H, d, J=9.6 Hz), 6.93 (1H, br, s), 7.34 (5H, s), 7.87 (1H, s), 8.46 (1H, s); API-ES/MS: 521[M+H]⁺, 543[M+Na]⁺.

### EXAMPLE 151

Ethyl 2-(2,2-diacetylvinyl)-5-(1-isopropyl-6-oxo-1,6-dihydro-3-pyridazinyl)-6-phenyl-3-pyridylcarbamate was prepared in a similar manner to that of Example 150.

¹H NMR (DMSO-d₆, δ): 1.04 (6H, d, J=6.50 Hz), 1.31 (3H, t, J=7.08 Hz), 2.15 (3H, s), 2.51 (3H, s: overlapped with signals of DMSO), 4.22 (2H, q, J=7.08 Hz), 5.05 (1H, 7-plet, J=6.50 Hz), 6.87 (1H, d, J=9.62 Hz), 7.23-7.40 (6H, m), 7.81 (1H, s), 8.33 (1H, s), 10.10 (1H, s)

### EXAMPLE 152

To a solution of 2-formyl-3-ethoxycarbonylamino-5-(2-isopropyl-3 (2H)-pyridazinon-6-yl)-6-phenylpyridine (406.5 mg) and 1,3-cyclopentandione (127.5 mg) in MeOH (8 ml) was added a catalytic amount of piperidine. The mixture was refluxed under stirring for 7 hours and allowed to stand at ambient temperature overnight. MeOH was removed in vacuo to give a residue, to which was added water under stirring. The mixture was sonicated to afford precipitate, which was collected by filtration, washed with water and dried to give white powder. This was purified by subjecting to column chromatography on silica gel eluting with a mixture of CHCl₃ and MeOH (30:1) to give 2-bis(cyclopentan-1,3-dion-2-yl)methyl-3-ethoxycarbonylamino-5-(2-isopropyl-3 (2H)-pyridazinon-6-yl)-6-phenylpyridine (247.4 mg) as white crystalline powder.

¹H NMR (DMSO-d₆, δ): 1.00 (6H, d, J=6.6 Hz), 1.30 (3H, t, J=7.1 Hz), 2.34 (8H, s), 4.19 (2H, q, J=7.1 Hz), 5.03 (1H, 7-plet, J=6.6 Hz), 5.34 (1H, s), 6.86 (1H, d, J=9.6 Hz), 7.25 (1H, d, J=9.6 Hz), 7.25-7.5 (5H, m), 8.30 (1H, d, J=5.1 Hz), 9.79 (1H, br, s); API-ES/MS: 585[M+H]⁺, 607[M+Na]⁺; API-ES, Negative/MS: 583[M−H]⁺.

### EXAMPLE 153

Ethyl 2-[(2,6-dioxocyclohexylidene)methyl]-5-(1-isopropyl-6-oxo-1,6-dihydro-3-pyridazinyl)-6-phenyl-3-pyridylcarbamate was prepared in a similar manner to that of Example 150. ¹H NMR (CDCl₃, δ): 1.23 (3H, d, J=6.6 Hz), 1.30 (3H, d, J=6.6 Hz), 1.38 (3H, t, J=7.2 Hz), 1.6-2.5 (6H, m), 4.28 (2H, q, J=7.2 Hz), 5.28 (1H, 7-plet, J=6.6 Hz), 5.66 (1H, s), 6.72 (1H, d, J=9.5 Hz), 6.93 (1H, d, J=9.5 Hz), 7.2-7.5 (5H, m), 8.48 (1H, s), 10.71 (1H, br, s); API-ES/MS: 501[M+H]⁺, 523[M+Na]⁺; APCI, Negative/MS: 499[M−H]⁺.

### EXAMPLE 154

Ethyl 2-[(E)-(2-amino-4-oxo-1,3-thiazol-5 (2H)-ylidene)methyl]-5-(1-isopropyl-6-oxo-1,6-dihydro-3-pyridazinyl)-6-phenyl-3-pyridylcarbamate

¹H NMR (DMSO-d₆, δ): 1.03 (6H, d, J=6.6 Hz), 1.29 (3H, t, J=7.1 Hz), 4.19 (2H, q, J=7.1 Hz), 5.05 (1H, 7-plet, J=6.6 Hz), 6.87 (1H, d, J=9.6 Hz), 7.29 (1H, d, J=9.6 Hz), 7.35-7.5 (5H, m), 7.87 (1H, s), 8.18 (1H, s), 9.1-9.3 (1H, br, s), 9.8-10.1 (1H, br, s); API-ES/MS: 505[M+H]⁺, 527[M+Na]⁺.

### EXAMPLE 155

A mixture of 2-formyl-3-ethoxycarbonylamino-5-(2-isopropyl-3(2H)-pyridazinon-6-yl)-6-phenylpyridine (406.5 mg), hydroxylamine hydrochloride (90.4 mg) and NaOAc (115 mg) in AcOH (10 ml) was stirred for 3 hours at ambient temperature. AcOH was removed in vacuo as much as possible and to the residue was added water. The mixture was made basic with a saturated aq.NaHCO₃ solution under stirring. The resultant precipitate was collected by filtration, washed with water and dried to give 2-hydroxyiminomethyl-3-ethoxycarbonylamino-5-(2-isopropyl-3(2H)-pyridazinon-6-yl)-6-phenylpyridine (421.0 mg) as yellow crystal.

₁H NMR (DMSO-d₆, δ): 1.08 (6H, d, J=6.58 Hz), 1.28 (3H, t, J=7.06 Hz), 4.23 (2H, q, J=7.06 Hz), 5.08 (1H, 7-plet, J=6.58 Hz), 6.85 (1H, d, J=9.62 Hz), 7.21 (1H, d, J=9.62 Hz), 7.31-7.40 (5H, m), 8.36 (1H, s), 8.83 (1H, s), 10.27 (1H, s), 12.24 (1H, s); API-ES/MS: 422[M+H]⁺, 444[M+Na]⁺.

### EXAMPLE 156

To a solution of E-, Z-mixture of 2-hydroxyiminomethyl-3-ethoxycarbonylamino-5-(2-isopropyl-3(2H)-pyridazinon-6-yl)-6-phenylpyridine (84.3 mg) and pyridine (39.5 mg) in CH₂Cl₂ (3 ml) was added benzoyl chloride (42.2 mg) under stirring at ambient temperature. The mixture was stirred for 20 hours and evaporated in vacuo. The residue was extracted with EtOAc and washed with water twice. After drying over MgSO₄, the solvent was removed in vacuo to give an oil (0.12 g), which was subjected to preparative thin layer chromatography on silica gel developing with a mixture of CHCl₃ and MeOH (30:1). The desired product, an E-, Z-mixture (ca 2:1) of 2-benzoyloxyiminomethyl-3-ethoxycarbonylamino-5-(2-isopropyl-3 (2H)-pyridazinon-6-yl)-6-phenylpyridine (83.1 mg), was obtained as an amorphous mass.

¹H NMR (DMSO-d₆, δ): 1.08 (6H, d, J=6.56 Hz), 1.27 and 1.30 (3H, each t, each J=6.68 Hz), 4.22 and 4.26 (2H, each q, each J=6.68 Hz), 5.08 (1H, 7-plet, J=6.56 Hz), 6.88 and 6.89 (ca 1:2)(1H, each d, each J=9.56 Hz), 7.26 and 7.29 (ca 1:2) (1H, each d, each J=9.56 Hz), 7.3-8.2 (9H, m), 8.32 and 9.13 (ca 1:2)(1H, each s), 8.82 (1H, d, J=7.82 Hz), 10.03 and 10.10 (ca 1:2)(1H, each s); API-ES/MS: 526[M+H]⁺, 548[M+Na]⁺.

### EXAMPLE 157

2-Phenylacetoxyiminomethyl-3-ethoxycarbonylamino-5-(2-isopropyl-3 (2H)-pyridazinon-6-yl)-6-phenylpyridine was prepared in a similar manner to that of Example 156.

¹H NMR (DMSO-d₆, δ): 1.06 (6H, d, J=6.42 Hz), 1.24 and 1.28 (3H, each t, each J=7.0 Hz), 3.97 (2H, s), 4.15-4.26 (2H, m), 5.04 (1H, 7-plet, J=6.42 Hz), 6.87 (1H, d, J=9.6 Hz), 7.25-7.40 (9H, m), 8.79-8.82 (1H, m), 8.85 (1H, s), 10.00 (1H, s); API-ES/MS: 540[M+H]⁺, 562[M+Na]⁺.

### EXAMPLE 158

To a suspension of cyanomethyltriphenylphosphonium chloride (405 mg) in THF (10 ml) was added potassium tert-butoxide (135 mg) under stirring at ambient temperature. The mixture was stirred for 0.5 hour under the same conditions. To the mixture was added 2-formyl-3-ethoxycarbonylamino-5-(2-isopropyl-3 (2H)-pyridazinon-6-yl)-6-phenylpyridine (406.5 mg) at once. The mixture was stirred for 1.5 hour at ambient temperature. THF was removed to give a residue, to which was added water and the mixture was extracted with EtOAc. The extract was washed with water twice and dried over MgSO₄. Removal of the solvent afforded an oil, which was subjected to column chromatography on silica gel eluting with a mixture of CHCl₃ and MeOH (100:1). The desired fractions were combined and evaporated in vacuo gave a yellow oil, which was triturated in IPE to afford white crystalline powder. Collection by filtration and drying gave E, Z-mixture (ca. 1:1) of 2-cyanovinyl-3-ethoxycarbonylamino-5-(2-isopropyl-3 (2H)-pyridazinon-6-yl)-6-phenylpyridine (282.0 mg, A) contaminated with triphenylphosphine oxide. The filtrate was concentrated in vacuo to give a yellow oil of z-derivative (439.8 mg, B), which was also contaminated with triphenylphosphine oxide.

A: ¹H NMR (CDCl₃, δ): 6.72 (1H, d, J=11.66 Hz) owing to vinyl proton of E-form; API-ES/MS: 430[M+H]⁺, 452[M+Na]⁺; B: ¹H NMR (CDCl₃, δ): 6.83 (1H, d, J=5.14 Hz) owing to vinyl proton of Z-form.

### EXAMPLE 159

Ethyl (2E)-3-[3-[(ethoxycarbonyl)amino]-5-(1-isopropyl-6-oxo-1,6-dihydro-3-pyridazinyl)-6-phenyl-2-pyridyl]acrylate was prepared in a similar manner to that of Example 138.

¹H NMR (CDCl₃, δ): 1.27 (6H, d, J=6.66 Hz), 1.35 (3H, t, J=7.10 Hz), 1.37 (3H, t, J=7.10 Hz), 4.30 (4H, q, J=7.10 Hz), 5.29 (1H, 7-plet, J=6.66 Hz), 6.71 (1H, d, J=9.58 Hz), 6.89 (1H, d, J=9.58 Hz), 6.90 (1H, br, s), 7.20 (1H, d, J=15.08 Hz), 7.32-7.44 (5H, m), 7.89 (1H, d, J=15.08 Hz), 8.44 (1H, s); API-ES/MS: 477[M+H]⁺, 499[M+Na]⁺.

### EXAMPLE 160

A mixture of 6-(6-amino-5-chloro-2-phenyl-3-pyridyl)-2-isopropyl-3 (2H)-pyridazinone (200 mg) and 75%m-chloroperbenzoic acid (149 mg) in DMF (30 ml) in CH₂Cl₂ was stirred at 25° C. After 3 hours, aq.NaHCO₃ solution was added to the reaction mixture, which was extracted with EtOAc. The EtOAc phase was separated, dried over earth granular. The solvent was removed in vacuo to give an oily residue. The residue was purified by silica gel column chromatography eluted with a mixture of CHCl₃ and MeOH. The fractions were concentrated in vacuo to obtain 6-(6-amino-5-chloro-1-oxido-2-phenyl-3-pyridyl)-2-isopropyl-3 (2H)-pyridazinone (40 mg) as white powder.

¹H NMR (DMSO-d₆, δ): 1.01 (6H, d, J=6.6 Hz), 4.96 (1H, 7-plet, J=6.6 Hz), 6.73 (1H, d, J=9.6 Hz), 7.11 (1H, d, J=9.6 Hz), 7.2-7.5 (7H, m), 7.62 (1H, s); API-ES/MS: 357[M+H]⁺, 359[M+2+H]⁺, 379[M+Na]⁺, 381[M+2+Na]⁺.

### EXAMPLE 161

A mixture of 6-(6-amino-5-chloro-2-phenyl-3-pyridyl)-3 (2H)-pyridazinone (300 mg) and sodium hydride (42.2 mg) in DMF (3 ml) was stirred at 25° C. After 1 hour, 1-methoxy-2-propyl methanesulfonate(186 g) was added to the reaction mixture, which was stirred at 25° C. for 14 hours. Water was added to the reaction mixture, which was extracted with EtOAc. The EtOAc phase was separated, dried over earth granular. The solvent was removed in vacuo to give oily residue. The residue was purified by silica gel column chromatography eluted with a mixture of CHCl₃ and MeOH. The fractions were concentrated in vacuo to obtain 6-(6-amino-5-chloro-2-phenyl-3-pyridyl)-2-(2-methoxy-1-methylethyl)-3 (2H)-pyridazinone (200 mg) as white powder.

¹H NMR (DMSO-d₆, δ): 0.97 (6H, d, J=6.8 Hz), 3.16 (3H, s), 3.2-3.5 (2H, m), 5.0-5.3 (1H, m), 6.7-6.9 (3H, m), 7.10 (1H, d, J=9.6 Hz), 7.2-7.4 (5H, m), 7.79 (1H, s); API-ES/MS: 371[M+H]⁺, 373[M+2+H]⁺, 393[M+Na]⁺, 395[M+2+Na]⁺.

### EXAMPLE 162

6-(6-Amino-5-chloro-2-phenyl-3-pyridyl)-2-(2-hydroxy-1-methylethyl)-3(2H)-pyridazinone was prepared in a similar manner to that of Example 107.

¹H NMR (DMSO-d₆, δ): 0.98 (6H, d, J=6.7 Hz), 3.2-3.6 (2H, m), 4.72 (1H, t, J=5.8 Hz), 4.85-5.1 (1H, m), 6.6-6.8 (3H, m), 7.01 (1H, d, J=9.6 Hz), 7.2-7.4 (5H, m), 7.80 (1H, s); API-ES/MS: 357[M+H]⁺, 379[M+Na]⁺.

1. A pyridazinone or pyridone compound of the following formula (I) 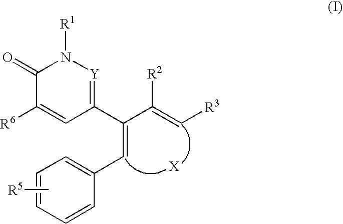
wherein
X is 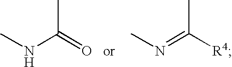
Y is N or CH;
R¹ is hydrogen or optionally substituted lower alkyl;
R² is hydrogen or halogen;
R³ is hydrogen, lower alkyl, lower alkoxy, halogen, hydroxy, cyano, amino, carbamoyl, thiocarbamoyl, aryl, acyl, acylamino or heterocyclic group, each of which may be optionally substituted;
R⁴ is hydrogen, lower alkyl, lower alkoxy, halogen, hydroxy, cyano, amino, carbamoyl, acyl, acylamino or
—A—R⁷
wherein
A is —CH═CH— or —CH═N—, and
R⁷ is lower alkyl, lower alkoxy, hydroxy, cyano, acyl, aryl(lower)alkoxy or acyloxy, each of which may be optionally substituted;
R⁵ is hydrogen, lower alkyl, lower alkoxy, halogen, hydroxy, each of which may be optionally substituted; and
R⁶ is hydrogen or halogen;
or a salt thereof.

2. A compound of claim 1,
wherein
Y is N;
R¹ is hydrogen, lower alkyl, aryl(lower)alkyl, or
—(CH₂)ₙ—CO—R⁸
wherein
n is 1 or 2, and
R⁸ is hydroxy, lower alkoxy, aryl, amino, lower alkylamino, hydroxy(lower)alkylamino or optionally substituted heterocyclic group;
R² is hydrogen;
R³ is hydrogen, lower alkyl, hydroxy(lower)alkyl, lower alkoxy, amino(lower)alkoxy, halogen, hydroxy, cyano, amino, carboxy, lower alkylaminocarbonyl, lower alkanoyl, lower alkoxycarbonyl, lower alkoxycarbonylamino, carbamoyl(lower)alkoxy, optionally subsituted heterocyclic group or optionally substituted heterocyclic carbonyl;
R⁴ is hydrogen, lower alkyl, lower alkoxy, optionally substituted amino(lower)alkoxy, halogen, hydroxy, cyano, amino, hydrazino, carbamoyl, carbamoyl(lower)alkoxy, carboxy, lower alkanoyl, lower alkoxycarbonyl, aryl(lower)alkylamino, heterocyclic(lower)alkylamino, heterocyclic(lower)alkoxy, or
—NH—CO—R⁹
wherein
R⁹ is lower alkyl, lower alkoxy, aryl or heterocyclic group;
R⁵ is hydrogen, lower alkoxy, hydroxy or halogen; and
R⁶ is hydrogen;
or a salt thereof.

3. A compound of claim 1,
wherein
Y is CH;
R¹ is hydrogen or lower alkyl,
R² is hydrogen or halogen;
R³ is hydrogen, halogen or amino;
R⁴ is hydrogen, halogen or amino;
R⁵ is hydrogen; and
R⁶ is hydrogen or halogen;
or a salt thereof.

4. A compound of claim 2,
wherein
X is 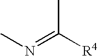
R¹ is hydrogen, lower alkyl;
R³ is hydrogen, hydroxy(lower)alkyl, halogen, hydroxy, amino, lower alkylaminocarbonyl, lower alkoxycarbonyl, optionally subsituted heterocyclic group or optionally substituted heterocyclic carbonyl; and
R⁴ is hydrogen, halogen, amino, hydrazino, aryl(lower)alkylamino, heterocyclic(lower)alkylamino, heterocyclic(lower)alkoxy, or
—NH—CO—R⁹
wherein
R⁹ is lower alkyl, aryl or heterocyclic group;
or a salt thereof.

5. A compound of claim 4,
wherein
R¹ is hydrogen, methyl, ethyl or isopropyl;
R³ is hydrogen, hydroxymethyl, chloro, bromo, iodo, hydroxy, methoxycarbonyl, methylthiazolyl or methylpyrazolyl;
R⁴ is hydrogen, chloro, bromo, iodo, amino, hydrazino, benzylamino, pyridylmethyl, acetamido, tert-butylcarbonylamino or benzoylamino; and
R⁵ is hydrogen, methoxy, hydroxy, fluoro, chloro, bromo or iodo;
or a salt thereof.

6. A compound of claim 3,
wherein
R¹ is isopropyl,
R² is hydrogen or chloro;
R³ is hydrogen, chloro or amino;
R⁴ is chloro or amino;
R⁶ is hydrogen or chloro;
or a salt thereof.

7. A compound of claim 5,
wherein
R¹ is isopropyl;
R³ is hydrogen, chloro, hydroxy, methoxycarbonyl or methylthiazolyl;
R⁴ is hydrogen, chloro, amino, hydrazino, benzylamino, pyridylmethyl, acetamido or benzoylamino; and
R⁵ is hydrogen, hydroxy, fluoro or chloro;
or a salt thereof.

8. A compound of claim 4,
wherein
R³ is hydrogen, halogen or hydroxy; and
R⁴ is hydrogen, amino;
or a salt thereof.

9. A compound of claim 8,
wherein
R¹ is lower alkyl,
R³ is hydrogen or halogen; and
R⁵ is hydrogen or halogen;
or a salt thereof.

10. A process for the preparation of the pyridazinone or pyridone compound of claim 1 or a salt thereof, which comprises,
(1) reacting a compound of the formula (II): 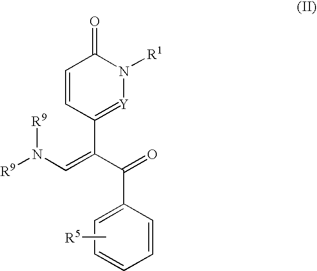
wherein
R¹, R⁵ and Y are each as defined above, and R⁹ is lower alkyl or a salt thereof, with 2-cyanoacetamide to give a compound of the formula (Iab): 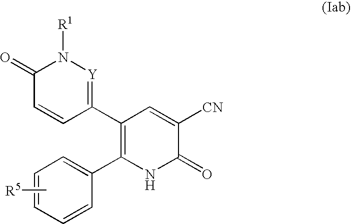
wherein R¹, R⁵ and Y are each as defined above or a salt thereof,
(2) reacting a compound of the formula (Iaa): 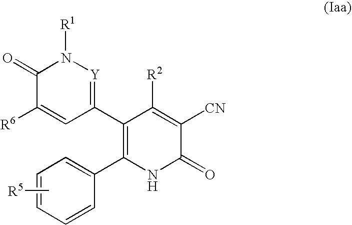
wherein R¹, R², R⁵ R⁶ and Y are each as defined above or a salt thereof, with a compound of the formula (III):
PO(—Hal)₃ (III)
wherein Hal is a halogen atom,
to give a compound of the formula (Ibb): 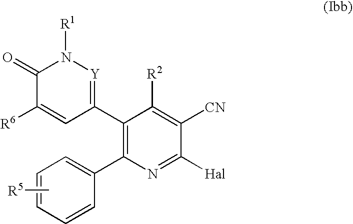
wherein R¹, R², R⁵, R⁶, Y and Hal are each as defined above or a salt thereof,
(3) hydrating a compound of the formula (Ic): 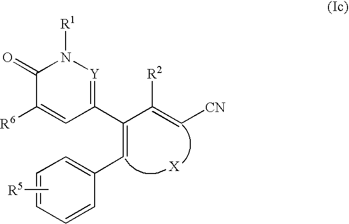
wherein R¹, R², R⁵, R⁶, X and Y are each as defined above or a salt thereof,
to give a compound of the formula (Id): 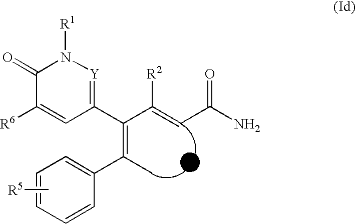
wherein R¹, R², R⁵, R⁶, X and Y are each as defined above or a salt thereof,
(4) dehydrating a compound (Id) or a salt thereof above to give a compound (Ic) or a salt thereof above,
(5) dehalogenating or esterificating a compound of the formula (Ib): 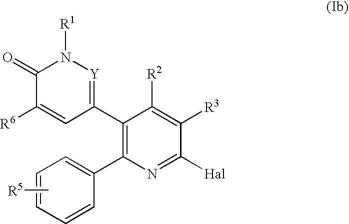
wherein R¹, R², R³, R⁵, R⁶, X, Y and Hal are each as defined above or a salt thereof,
with a compound of the formula (IV):
Z—R^{4a} (IV)
wherein Z is hydrogen or alkali metal, and R^{4a} is the same as R⁴ defined above exept for halogen, or a salt thereof,
to give a compound of the formula (Ie): 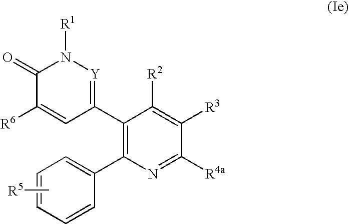
wherein R¹, R², R³, R^{4a}, R⁵, R⁶, X and Y are each as defined above or a salt thereof,
(6) carboxylating a compound (Ic) or a salt thereof above to give a compound of the formula (Ifa): 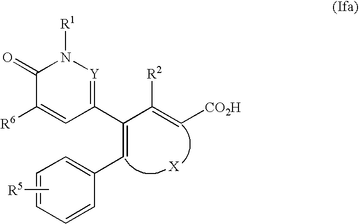
wherein R¹, R², R⁵, R⁶, X and Y are each as defined above or a salt thereof,
(7) hydrolyzing a compound (Id) or a salt thereof above to give a compound (Ifa) or a salt thereof above,
(8) esterificating a compound (Ifa) or a salt thereof above to give a compound of the formula (If): 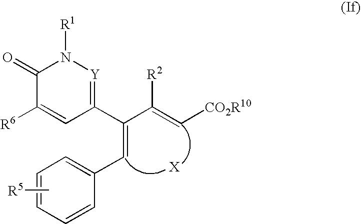
wherein R¹, R², R⁵, R⁶, X and Y are each as defined above, and R¹⁰ is lower alkyl or a salt thereof,
(9) hydrolyzing a compound of the formula (Ig): 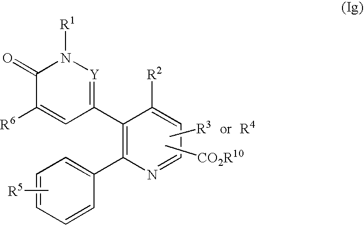
wherein R¹, R², R³, R⁴, R⁵, R⁶, R¹⁰, X and Y are each as defined above or a salt thereof, to give a compound of the formula (Iga): 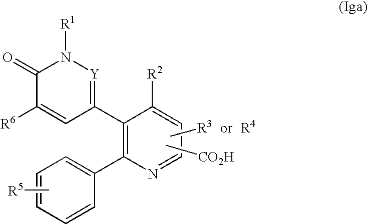
wherein R¹, R², R³, R⁴, R⁵, R⁶, X and Y are each as defined above or a salt thereof,
(10) amidating a compound (Iga) or a salt thereof above to give a compound of the formula (Iha) or the formula (Ihb): 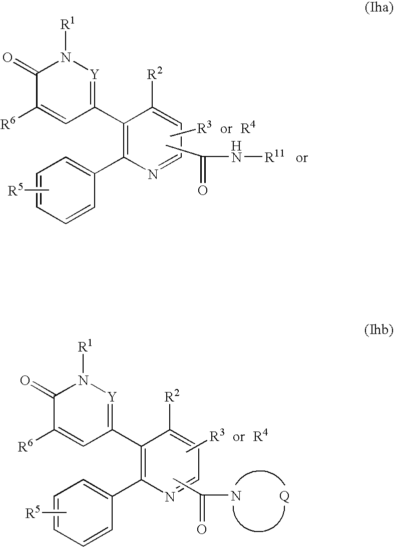
wherein R¹, R², R³, R⁴, R⁵, R⁶ and Y are each as defined above, and R¹¹ is optionally substituted lower alkyl, and 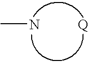
is optionally substituted heteromonocyclic group containing nitrogen atom(s) or a salt thereof,
(11) substituting carboxy group of a compound (Ifa) or a salt thereof above with acylamino group to give a compound of the formula (Iia): 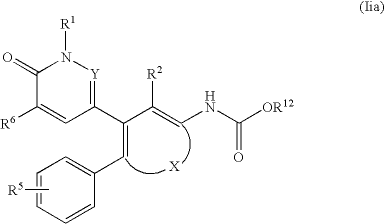
wherein R¹, R², R⁵, R⁶, X and Y are each as defined above, and R¹² is a lower alkyl or a salt thereof,
(12) hydrolyzing a compound (Iia) or a salt thereof above to give a compound of the formula (Ii): 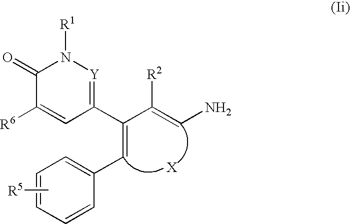
wherein R¹, R², R⁵, R⁶, X and Y are each as defined above or a salt thereof,
(13) dehydrogenating a compound of the formula (V): 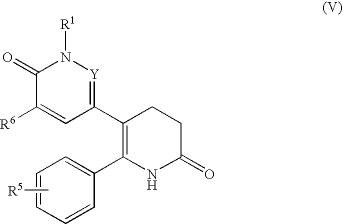
wherein R¹, R⁵, R⁶ and Y are each as defined above or a salt thereof to give a compound of the formula (Iac): 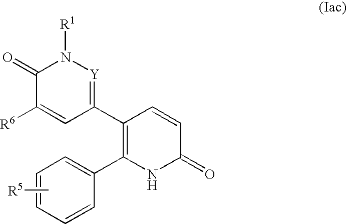
wherein R¹, R⁵, R⁶ and Y are each as defined above or a salt thereof,
(14) alkylating the oxygen atom of the compound (Iac) or a salt thereof above to give a compound of the formula (Ij): 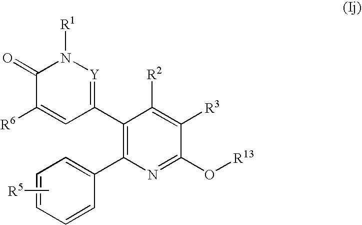
wherein R¹, R², R³, R⁵, R⁶ and Y are each as defined above, and R¹³ is optionally substituted lower alkyl or a salt thereof,
(15) amidating a compound of the formula (Ija): 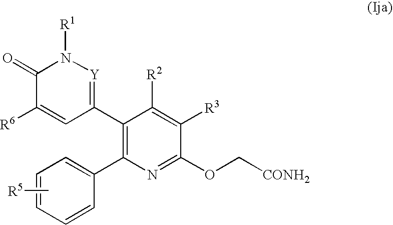
wherein R¹, R², R³, R⁵, R⁶ and Y are each as defined above or a salt thereof to give a compound of the formula (Ik): 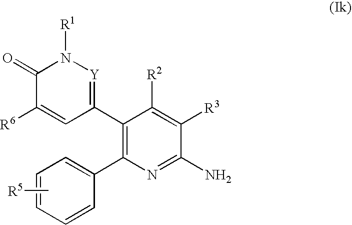
wherein R¹, R², R³, R⁵, R⁶ and Y are each as defined above or a salt thereof,
(16) amidating a compound of the formula (Il): 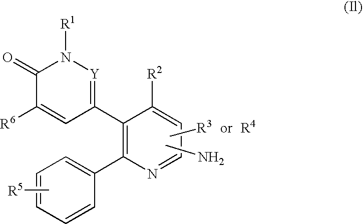
wherein R¹, R², R³, R⁴, R⁵, R⁶ and Y are each as defined above or a salt thereof to give a compound of the formula (Ila): 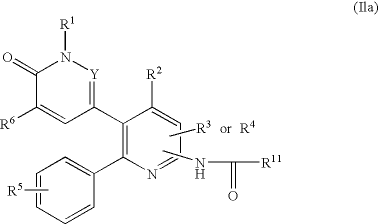
wherein R¹, R², R³, R⁴, R⁵, R⁶ and Y are each as defined above, and R¹⁴ is optionally substituted aryl or heterocyclic group, or a salt thereof,
(17) reacting a compound of the formula (Im): 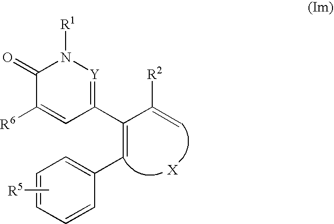
wherein R¹, R², R⁵, R⁶, X and Y are each as defined above or a salt thereof with a compound of the formula (VI): 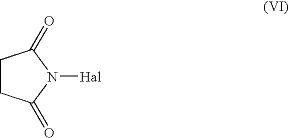
wherein Hal is as defined above to give a compound of the formula (In): 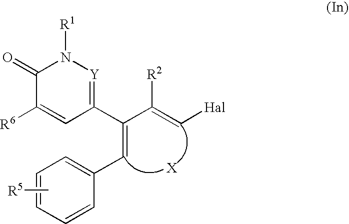
wherein R¹, R², R⁵, R⁶, X, Y and Hal are each as defined above or a salt thereof,
(18) reacting a compound of the formula (Io): 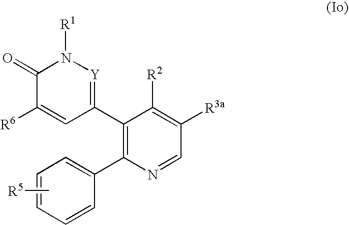
wherein R¹, R², R⁵, R⁶ and Y are each as defined above, and R^{3a} is the same as R³defined above except for hydrogen or a salt thereof with a compound (VI) above to give a compound of the formula (Iba): 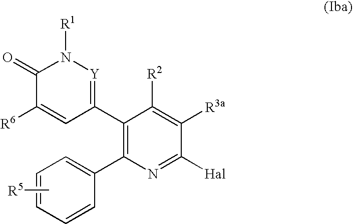
wherein R¹, R², R^{3a}, R⁵, R⁶, Y and Hal are each as defined above or the salt thereof,
(19) reacting a compound of the formula (Ip): 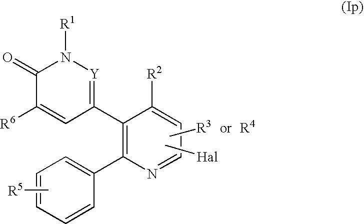
wherein R¹, R², R³, R⁴, R⁵, R⁶, Y and Hal are each as defined above or a salt thereof with a compound of the formula (VII): 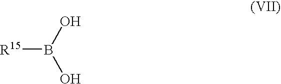
wherein R¹⁵ is optionally substituted aryl or heterocyclic group or a salt thereof to give a compound of the formula (Iq): 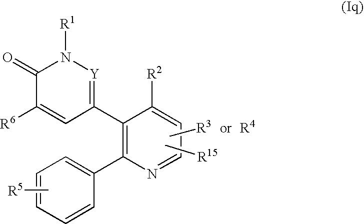
wherein R¹, R², R³, R⁴, R⁵, R⁶, R¹⁵ and Y are each as defined above or a salt thereof,
(20) decarboxylating a compound (Ifa) or a salt thereof above to give a sompound of the formula (Im) or a salt thereof above,
(21) hydroxylating a compound (Ii) or a salt thereof above to give a compound of the formula (Ira): 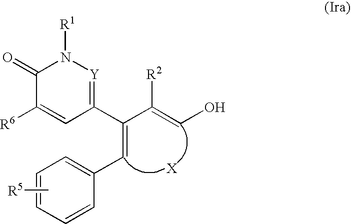
wherein R¹, R², R⁵, R⁶, R¹⁵ and Y are each as defined above or a salt thereof,
(22) alkylating an oxygen atom of the compound (Ira) or a salt thereof above to give a compound of the formula (Ir): 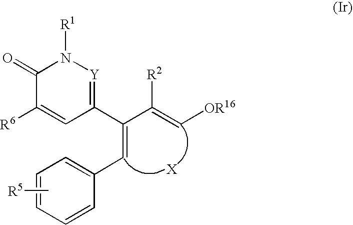
wherein R¹, R², R⁵, R⁶ and Y are each as defined above, and R¹⁶ is optionally substituted lower alkyl or a salt thereof,
(23) subjecting a compound (Il) or a salt thereof above to reductive amination with a compound of the formula (VIII): 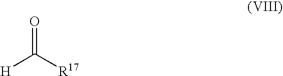
wherein R¹⁷ is optionally substituted aryl or heterocyclic group or a salt thereof to give a compound of the formula (Ilb): 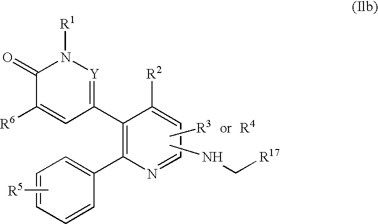
wherein R¹, R², R³, R⁴, R⁵, R⁶, R¹⁷ and Y are each as defined above or a salt thereof,
(24) hydrolyzing a compound of the formula (IX): 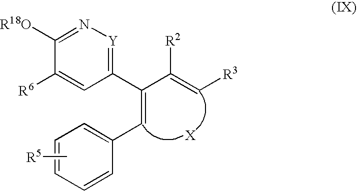
wherein R², R³, R⁵, R⁶, X and Y are each as defined above, and R¹⁸ is a lower alkyl or a salt thereof to give a compound of the formula (Is): 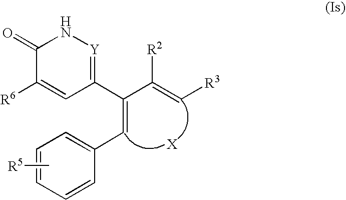
wherein R², R³, R⁵, R⁶, X and Y are each as defined above or a salt thereof,
(25) alkylating a nitrogen atom of the compound (Is) or a salt thereof above to give a compound of the formula (I′): 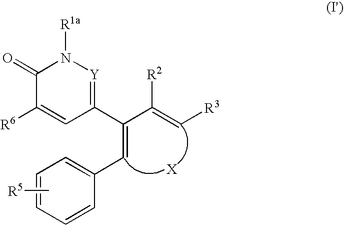
wherein R², R³, R⁵, R⁶, X and Y are each as defined above, and R^{1a} is the same as R¹ defined above except for hydrogen or a salt thereof,
(26) hydrolyzing a compound of the formula (It): 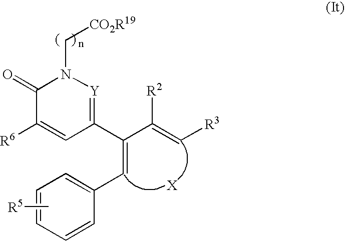
wherein R², R³, R⁵, R⁶, X and Y are each as defined above, R¹⁹ is optionally substituted lower alkyl and n is 1 or 2, or a salt thereof to give a compound of the formula (Ita): 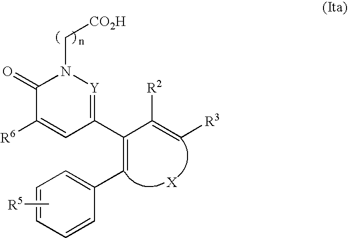
wherein R², R³, R⁵, R⁶, X, Y and n are each as defined above or a salt thereof,
(27) amidating a compound (Ita) or a salt thereof above to give a compound of the formula (Iua) or the formula (Iub): 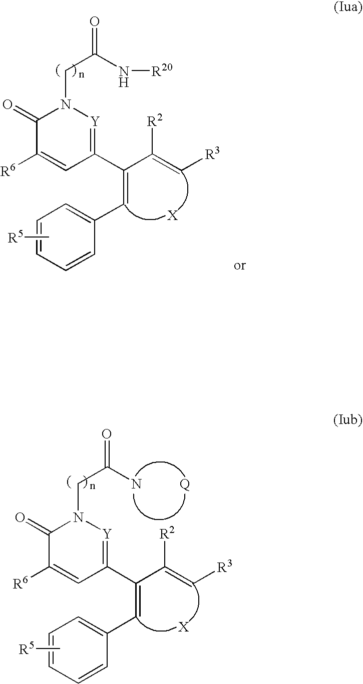
wherein R², R³, R⁵, R⁶ X, Y, n and 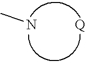
are each as defined above, and R²⁰ is hydrogen or optionally substituted lower alkyl, or a salt thereof,
(28) eliminating of alkyl group of a compound of the formula (Iv): 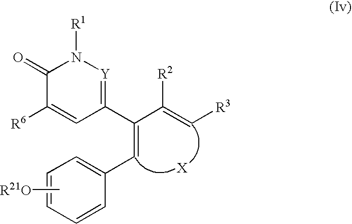
wherein R¹, R², R³, R⁶, X, and Y are each as defined above, R²¹ is a lower alkyl or a salt thereof, to give a compound of the formula (Iva): 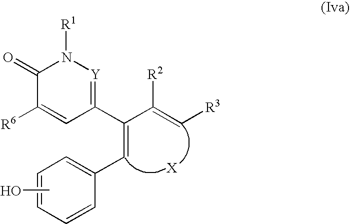
wherein R¹, R², R³, R⁶, X, and Y are each as defined above or a salt thereof,
(29) dehydrogenating of a compound of the formula (X): 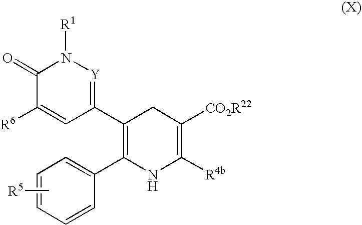
wherein R¹, R⁵, R⁶ and Y are each as defined above, R^{4b} is optionally substituted lower alkyl and R²² is a lower alkyl or a salt thereof, to give a compound of the formula (Iw): 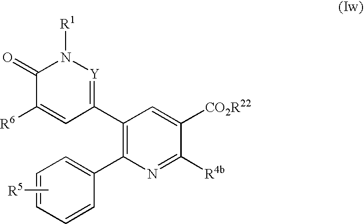
wherein R¹, R^{4b}, R⁵, R⁶, R²² and Y are each as defined above or a salt thereof,
(30) hydrolyzing a compound of the formula (Ix): 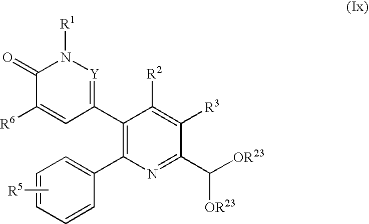
wherein R¹, R², R³, R⁵, R⁶ and Y are each as defined above, and R²³ is lower alkyl or a salt thereof to give a compound of the formula (Iy): 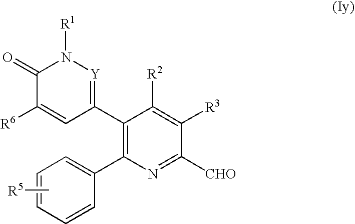
wherein R¹, R², R³, R⁵, R⁶ and Y are each as defined above or a salt thereof,
(31) reacting a compound (Iy) or a salt thereof above with hydroxylamine in the presence of sodium acetate, following to hydrolysis to give a compound of the formula (Iza): 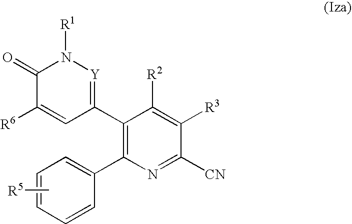
wherein R¹, R², R³, R⁵, R⁶ and Y are each as defined above or a salt thereof,
(32) subjecting a compound (Iy) or a salt thereof above to olefin formating reaction to give a compound of the formula (Izb): 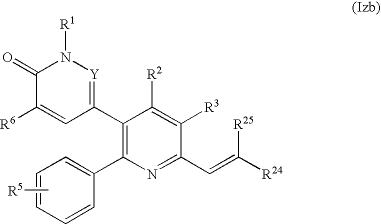
wherein R¹, R², R³, R⁵, R⁶ and Y are each as defined above, and R²⁴ and R²⁵ are each independently hydrogen or the same as R⁷ defined above,
(33) reacting the compound (Iy) or a salt thereof above with N-optionally substituted hydroxylamine to give a compound of the formula (Izc): 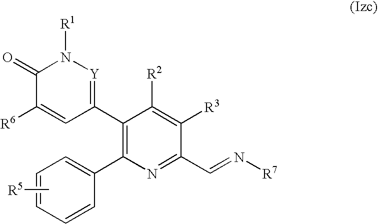
wherein R¹, R², R³, R⁵, R⁶, R⁷ and Y are each as defined above or a salt thereof,
(34) subjecting a compound (Iy) or a salt thereof above to reductive amination to give a compound of the formula (Izd): 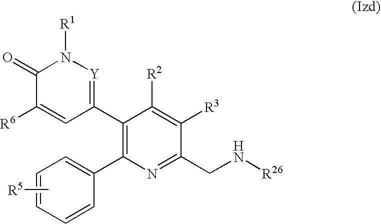
wherein R¹, R², R³, R⁵, R⁶ and Y are each as defined above, and R²⁶ is optionally substituted lower alkyl or a salt thereof.

11. A pharmaceutical composition comprising the compound of claim 1 or a pharmaceutically acceptable salt thereof in admixture with a pharmaceutically acceptable carrier.

12. A method for preventing or treating a disease selected from the group consisting of depression, dementia, Parkinson's disease, anxiety, pain, cerebrovascular disease, heart failure, hypertension, circulatory insufficiency, post-resuscitation, asystole, bradyarrhythmia, electromechanical dissociation, hemodynamic collapse, SIRS (systemic inflammatory response syndrome), multiple organ failure, renal failure (renal insufficiency), renal toxicity, nephrosis, nephritis, edema, obesity, bronchial asthma, gout, hyperuricemia, sudden infant death syndrome, immunosuppression, diabetes, ulcer, pancreatitis, Meniere's syndrome, anemia, dialysis-induced hypotension, constipation, ischemic bowel disease, ileus, myocardial infarction, thrombosis, obstruction, arteriosclerosis obliterans, thrombophlebitis, cerebral infarction, transient ischemic attack and angina pectoris, which comprises administering the compound of claim 1 or a pharmaceutically acceptable salt thereof to a human being or an animal.

13. A method for preventing or treating a disease selected from the group consisting of depression, dementia, Parkinson's disease, anxiety, pain, cerebrovascular disease, Meniere's syndrome and cerebral infarction, which comprises administering any of the compound of claim 1 to 9 or a pharmaceutically acceptable salt thereof to a human being or an animal.

14. A method for preventing or treating a disease selected from the group consisting of depression, dementia, Parkinson's disease and anxiety, which comprises administering any of the compound of claim 1 to 9 or a pharmaceutically acceptable salt thereof to a human being or an animal

15. Use of the compound of claim 1 or a pharmaceutically acceptable salt thereof as a medicament.

16. Use of the compound of claim 1 or a pharmaceutically acceptable salt thereof as an adenosine antagonist.

17. Use of the compound of claim 1 or a pharmaceutically acceptable salt thereof as an A₁ receptor and A₂ receptor dual antagonist.

18. A process for preparing a pharmaceutical composition which comprises admixing the compound of claim 1 or a pharmaceutically acceptable salt thereof with a pharmaceutically acceptable carrier.

19. Use of the compound of claim 1 or a pharmaceutically acceptable salt thereof for the production of a pharmaceutical composition for the therapy of diseases on which an adenosine antagonist is therapeutically effective.

20. A method for evaluation of adenosine antagonism which comprises use of compound of claim 1 or a pharmaceutically acceptable salt thereof.
